(19) United States
(12) Patent Application Publication
(10) Pub. No.: US 2019/0046724 A1
(43) Pub. Date: Feb. 14, 2019

(54) MODULAR DOSING ASSEMBLY OF MEDICAL SUBSTANCES

(71) Applicant: Mikael NAYGAUZ, Forrest Hills, NY (US)

(72) Inventor: Mikael NAYGAUZ, Forrest Hills, NY (US)

(21) Appl. No.: 16/102,681

(22) Filed: Aug. 13, 2018

Related U.S. Application Data
(60) Provisional application No. 62/545,152, filed on Aug. 14, 2017.
(60) Provisional application No. 62/649,483, filed on Mar. 28, 2018.
(60) Provisional application No. 62/660,885, filed on Apr. 20, 2018.
(60) Provisional application No. 62/666,866, filed on May 4, 2018.
(60) Provisional application No. 62/667,593, filed on May 6, 2018.
(60) Provisional application No. 62/670,833, filed on May 13, 2018.
(60) Provisional application No. 62/679,817, filed on Jun. 3, 2018.
(60) Provisional application No. 62/680,974, filed on Jun. 5, 2018.
(60) Provisional application No. 62/681,884, filed on Jun. 7, 2018.
(60) Provisional application No. 62/690,260, filed on Jun. 26, 2018.

(51) Int. Cl. A61M 5/178; A61M 5/162; A61M 5/19; A61J 1/10

(52) U.S. Cl. CPC A61M 5/1782; A61M 5/162; A61J 1/20; A61J 1/10; A61M 5/19

(57) ABSTRACT

Systems, devices and method to establish fluid communication between vessels. A vessel having at least two entry ports and at least one exit port, and at least one container containing a dosing regimen, the at least one container configured to be received by one of the at least two entry ports of the vessel, whereby upon connection of the at least one container to the one of the at least two entry ports of the vessel, the dosing regimen in the at least one container is transferred into the vessel. Modular dosing system for adding at least one amount of a medicament to a preparation in a modular construction. System for displaying a dosing regimen or single amount of a medicament, so that the administrator of the amount is able to precisely ascertain the amount administered to a patient.

### CROSS REFERENCE TO RELATED APPLICATIONS

This application claims the benefit of U.S. Provisional Patent Application No. 62/545,152 filed on Aug. 14, 2017; 62/558,502 filed on Sep. 14, 2017; 62/560,195 filed on Sep. 19, 2017; 62/562,490 filed on Sep. 25, 2017; 62/563,072 filed on Sep. 26, 2017; 62/564,933 filed on Sep. 28, 2017; 62/566,414 filed on Sep. 30, 2017; 62/582,922 filed on Nov. 7, 2017; 62/612,267 filed on Dec. 29, 2017; 62/619,795 filed Jan. 20, 2018; 62/619,920 filed Jan. 22, 2018; 62/625,334 filed on Feb. 1, 2018; 62/626,686 filed on Feb. 6, 2018; 62/631,729 filed on Feb. 17, 2018; 62/644,185 filed on Mar. 16, 2018; 62/649,483 filed on Mar. 28, 2018; 62/651,000 filed on Mar. 30, 2018; 62/651,149 filed on Mar. 31, 2018; 62/653,356 filed on Apr. 5, 2018; 62/657,806 filed on Apr. 14, 2018; 62/657,808 filed on Apr. 15, 2018; 62/657,809 filed on Apr. 15, 2018; 62/660,885 filed on Apr. 20, 2018; 62/662,721 filed on Apr. 25, 2018; 62/662,743 filed on Apr. 25, 2018; 62/664,933 filed on Apr. 30, 2018; 62/666,866 filed on May 4, 2018; 62/667,593 filed on May 6, 2018; 62/669,948 filed on May 10, 2018; 62/670,833 filed on May 13, 2018; 62/675,058 filed on May 22, 2018; 62/676,822 filed on May 25, 2018; 62/679,817 filed on Jun. 3, 2018; 62/680,576 filed on Jun. 4, 2018; 62/680,974 filed on Jun. 5, 2018; 62/681,884 filed on Jun. 7, 2018; 62/686,602 filed on Jun. 18, 2018; 62/686,612 filed on Jun. 18, 2018; and 62/690,260 filed on Jun. 26, 2018. The contents of the above applications are all incorporated by reference as if fully set forth herein in their entirety.

### FIELD OF THE INVENTION

The present invention relates, in some embodiments thereof, to systems, devices and substance transfer methods to establish fluid communication between medical vessels and devices. In some embodiments of the invention, the systems and devices of the invention include a vessel comprising at least two entry ports and at least one exit port, and at least one container containing an amount of a beneficial substance, the at least one container configured to be received by one of the at least two entry ports of the vessel, whereby upon connection of the at least one container to the one of the at least two entry ports of the vessel, the dosing regimen in the at least one container is transferred into the vessel, thereby allowing the user to assemble virtually any customized final amount for an individual with maximum flexibility and without manual manipulation of the beneficial substance, wherein the vessel may be a container or a syringe. In other embodiments of the invention, the systems and devices of the invention involve a modular dosing system for adding a plurality of amounts of medicaments in a modular construction. In other embodiments of the invention, the systems and devices of the invention include a system for displaying a dosing regimen or single amount of a medicament, so that the administrator of the amount is able to precisely ascertain the amount administered to a patient.

### BACKGROUND OF THE INVENTION

The medicinal practice routinely involves administration of medical substances, such as, medicaments, fluids, nutritional substances and the alike, to patients or animals. The preparation and/or administration of such medicinal substances typically involves one or more transfers of those substances between pharmaceutical vessels or administration devices (such as, vials, syringes, infusion lines, connectors, etc.). Each such act of transferring substances between vessels or devices exposes the connection interfaces of the vessels and accordingly the medical substances themselves to contaminants present in ambient air or ambient air particles (e.g., bacteria, viruses, funguses, spores, pyrogens, dirt). In addition, connection interfaces are further prone to contaminations due to physical contact of the interfaces, for example, with nonsterile gloves, or devices.

Such contaminations are a major problem in the healthcare setting since contaminants, once invading within medicinal substances, may pose substantial danger if administered intracorporeally to patients.

Typical connection interfaces of pharmaceutical vessels or pharmaceutical administration devices include rubber bungs and/or stoppers covered by a cap and/or seal that can be flicked off and/or are removed prior to usage thereof. These rubber bungs/stoppers are used to allow penetration by a needle attached to a syringe or by other medical connectors. When the cap and/or seal is flicked off and/or removed, the rubber bung and/or stopper is exposed to ambient air and to contaminants present therein. Accordingly, exposure of connection interfaces to ambient air may involve contamination of the interfaces and consequently contamination of a beneficial substance to be provided to a patient.

Existing systems include U.S. D720067; U.S. D717947; U.S. D703812; U.S. D690418; U.S. D639939; U.S. D637713; U.S. Pat. No. 9,790,011; U.S. Pat. No. 9,775,777; U.S. Pat. No. 9,561,326; U.S. Pat. No. 9,493,281; U.S. Pat. No. 9,492,353; U.S. Pat. No. 9,309,020; U.S. Pat. No. 9,173,816; U.S. Pat. No. 9,168,203; U.S. Pat. No. 9,162,803; U.S. Pat. No. 9,039,672; U.S. Pat. No. 8,926,583; U.S. Pat. No. 8,827,978; U.S. Pat. No. 8,790,330; U.S. Pat. No. 8,662,985; U.S. Pat. No. 8,657,803; U.S. Pat. No. 8,622,985; U.S. Pat. No. 8,562,583; U.S. Pat. No. 8,545,475; U.S. Pat. No. 8,523,838; U.S. Pat. No. 8,491,563; U.S. Pat. No. 8,480,646; U.S. Pat. No. 8,449,521; U.S. Pat. No. 8,381,776; U.S. Pat. No. 8,336,587; U.S. Pat. No. 8,328,772; U.S. Pat. No. 8,287,513; U.S. Pat. No. 8,225,826; U.S. Pat. No. 8,075,550; U.S. Pat. No. 8,029,747; U.S. Pat. No. 7,998,134; U.S. Pat. No. 7,975,733; U.S. Pat. No. 7,942,860; U.S. Pat. No. 7,867,215; U.S. Pat. No. 7,744,581; U.S. Pat. No. 7,731,678; U.S. Pat. No. 7,387,216; U.S. Pat. No. 7,306,584; U.S. Pat. No. 6,875,203; U.S. Pat. No. 6,729,370; U.S. Pat. No. 6,715,520; U.S. Pat. No. 6,602,239; U.S. Pat. No. 6,409,708; U.S. Pat. No. 6,343,629; U.S. Pat. No. 6,162,199; U.S. Pat. No. 6,113,583; U.S. Pat. No. 6,063,068; U.S. Pat. No. 5,893,397; U.S. Pat. No. 5,876,380; U.S. Pat. No. 5,832,971; U.S. Pat. No. 5,807,374; U.S. Pat. No. 5,746,733; U.S. Pat. No. 5,569,235; U.S. Pat. No. 5,462,535; U.S. Pat. No. 5,405,326; U.S. Pat. No. 5,292,318; U.S. Pat. No. 5,279,582; U.S. Pat. No. 4,944,723; U.S. Pat. No. 4,932,947; U.S. Pat. No. 4,932,937; U.S. Pat. No. 4,919,657; U.S. Pat. No. 4,915,701; U.S. Pat. No. 4,826,489; U.S. Pat. No. 4,673,404; U.S. Pat. No. 4,564,054; U.S. Pat. No. 3,610,241; U.S. Pat. No. 3,605,743; U.S. Pat. No. 3,587,575; U.S. Pat. No. 3,583,399; U.S. Pat. No. 3,578,037; U.S. Pat. No. 3,556,099; U.S. Pat. No. 3,552,387; U.S. Pat. No. 3,406,686; U.S. Pat. No. 3,380,450; U.S. Pat. No. 3,375,825; U.S. Pat. No. 3,342,180; U.S. Pat. No. 3,330,282; U.S. Pat. No. 3,330,281; U.S. Pat. No. 3,306,290; U.S. Pat. No. 3,255,752; U.S. Pat. No. 3,253,592; U.S. Pat. No. 3,076,456; U.S. Pat. No. 2,972,991; U.S. Pat. No. 2,922,419; US20160262982; US20160038373; US20150209568; US20140183196; US20140016570; US20140007973; US20140000754; US20130184672; US20130006200; US20120209238; US20120209218; US20120203194; US20110284561; US20110186177; US20110125128; US20110108158; US20110098647; US20100249745; US20100198182; US20100152669; US20100147402; US20100036319; US20100004602; US20090057258; US20080312634; US20080223484; US20080171981; US20060276759; US20050215976; US20030199847; US20030187420; US20020130100; US20020115981; US20020099354; ES2577377T3; EP2852367B1; EP2666513; EP2155141B1.

In order to overcome this obstacle, the current medical practice involves swabbing the surface of a connection interface with a disinfecting agent, such as 70% isopropyl alcohol, prior to accessing the connection interface. Other methods include i.v. (intravenous) rooms which are used for the sterile preparation of i.v. medications. Such rooms, to keep medicinal preparations as sterile as possible, are equipped with special instruments including, hoods with air filtration systems (e.g., HEPA filters), ventilation systems and air pressure systems. Additionally, those rooms necessitate that the medical staff working in these rooms are properly garmented, are properly trained, and require aseptic techniques, and employ quality control and validation processes. These systems require regular upkeep by certified personnel and require regular cleaning. These systems are therefore expensive, labor intensive, and require regular maintenance and testing to assure that they are operating effectively. The above described systems and methods are either cumbersome and expensive or inefficient in addressing the problem of reducing/eliminating contaminants on connection interfaces.

Taken together, the above described systems and methods are either cumbersome and expensive or inefficient in addressing the problem of reducing/eliminating contaminants on connection interfaces.

The other issue that was previously mentioned relates to standard commercially available amounts of beneficial substances and the disadvantage this poses for preparing tailored or customized final dosage amounts. The preparation of customized amounts of intracorporeally administered beneficial substances requires the manual manipulation of the beneficial substances typically by healthcare personnel. Manual manipulation involves drawing out from vials using syringes and needles of amounts of beneficial substances, measuring visually the amount of the beneficial substance that has been drawn into a syringe, and injecting the amount of the beneficial substance from a syringe into a second container, typically a bag or a bottle. The preparation of customized amount of beneficial substances typically involves using less than the amount provided in one commercially available container or typically involves using more than the amount provided in one commercially available container. Customized dosages, particularly of chemotherapeutic agents, are typically based on a patient's weight, height, and age. Therefore, the preparation of customized final amounts of beneficial substances require the manual manipulation and measurement of beneficial substances by healthcare personnel.

Thus, there is a long felt and unmet need for systems, devices and/or methods that afford transfer of medical substances in a sterile manner. There is a need for reliable, user friendly and cost-effective solutions allowing contaminant free engagement of vessels for drug preparation and administration processes. There is also a dire need for systems, devices and methods for the preparation of customized amounts of beneficial substances that obviate the need for manual manipulation of beneficial substances.

### SUMMARY OF THE INVENTION

Objects of the invention are achieved by providing systems, devices and methods for administering medical substances in a decontaminated manner.

Objects of the invention are achieved by providing systems, devices and methods which are directed to the transfer of medical substances in an efficient, user-friendly and essentially sterile manner.

Objects of the invention are achieved by providing systems, devices and methods which afford the transfer of medical substances in an efficient, user-friendly and essentially sterile manner and in a modular construction.

Objects of the invention are achieved by providing systems, devices and methods which afford the transfer of medical substances in an efficient, user-friendly and essentially sterile manner so that dosing regimens are clearly shown and are able to be ascertained to a person administering an amount to a patient.

The present invention provides devices and systems that decontaminate connection surfaces of medical or pharmaceutical vessels and thereafter allow decontaminated fluid passageway between the vessels.

In a first aspect, the present invention provides a system for adding at least one amount of a medicament to a vessel, the system comprising: a vessel including at least two entry ports; and at least one container housing an amount of a medicament; wherein the at least one container is configured to be directly received and engaged by one of the at least two entry ports of the vessel, wherein upon connection of the at least one container to the one of the at least two entry ports of the vessel, the amount of the medicament housed in the at least one container is transferred into the vessel.

In certain embodiments, the at least one container abuts the vessel when the at least one container is engaged to the vessel. In certain embodiments, the at least one container is flush mounted to the vessel when the at least one container is engaged to the vessel. In certain embodiments, the at least one container is surface mounted to the vessel when the at least one container is engaged to the vessel.

In certain embodiments, the amount of the medicament is a non-standard amount.

In certain embodiments, the at least one container is configured to be directly received by at least one of the at least two entry ports.

In certain embodiments, the vessel has a plurality of decontamination devices. In certain embodiments, the vessel is a bag or a bottle. In certain embodiments, the vessel is selected from a group consisting of a container, container with a flexible wall, container with a rigid wall, container with an expulsion member, a syringe, and container with a plunger.

In certain embodiments, the vessel is a cartridge or container based stacking system having a plurality of cartridges or containers. In certain embodiments, the amount of the medicament flows through the plurality of cartridges or containers.

In certain embodiments, the vessel includes at least two engagement mechanisms abutting a wall of the vessel. In certain embodiments, the at least two engagement mechanisms are surface mounted and/or flush mounted to a wall of the vessel. In certain embodiments, the at least two engagement mechanisms are configured to engage the at least two containers. In certain embodiments, the at least two engagement mechanisms are selected from a group consisting of a ratchet teeth mechanism, a snap-on mechanism, a slide-on mechanism, an adhesive mechanism, and combinations thereof. In certain embodiments, the at least two engagement mechanisms are a thread mechanism.

In certain embodiments, upon connection of the at least two containers to the vessel, the at least two containers abut a wall of the vessel. In certain embodiments, the upon connection of the at least two containers to the vessel, the at least two containers are surface mounted to a wall of the vessel. In certain embodiments, the upon connection of the at least two containers to the vessel, the at least two containers are flush mounted to a wall of the vessel. In certain embodiments, the at least two entry ports of the vessel abut the vessel. In certain embodiments, the at least two entry ports of the vessel abut a wall of the vessel. In embodiments, the at least two entry ports of the vessel are flush mounted the vessel. In certain embodiments, the at least two entry ports of the vessel are flush mounted to a wall of the vessel. In certain embodiments, the at least two entry ports of the vessel are surface mounted to the vessel. In certain embodiments, the at least two entry ports of the vessel are surface mounted to a wall of the vessel.

In certain embodiments, the vessel has at least three entry ports abutting the vessel. In certain embodiments, the vessel has at least four entry ports abutting a wall of the vessel. In certain embodiments, the vessel has at least three entry ports flush mounted to the vessel. In certain embodiments, the vessel has at least three entry ports flush mounted to a wall of the vessel. In certain embodiments, the vessel has at least four entry ports surface mounted to the vessel. In certain embodiments, the vessel has at least four entry ports surface mounted to a wall of the vessel.

In certain embodiments, the vessel is a container with a flexible wall. In certain embodiments, the vessel is a container is a bag. In certain embodiments, the vessel has a rigid wall. In certain embodiments, the vessel is a bottle. In certain embodiments, the vessel is a vial. In certain embodiments, the vessel is a cartridge. In certain embodiments, the vessel is a syringe.

In certain embodiments, the at least one container is at least two containers housing a medicament. In certain embodiments, the medicament is a beneficial substance.

In certain embodiments, the at least one container is at least three containers. In certain embodiments, the at least one container is four or more containers. In certain embodiments, at least two of the containers house a medicament. In certain embodiments, at least two of the containers house a beneficial substance. In certain embodiments, at least three of the containers house a medicament. In certain embodiments, at least three of the containers house a beneficial substance. In certain embodiments, at least four of the containers house a medicament. In certain embodiments, at least four of the containers house a beneficial substance.

In certain embodiments, at least two engagement mechanisms abut the vessel. In certain embodiments, at least two engagement mechanisms abut a wall of the vessel. In certain embodiments, at least three engagement mechanisms abut the vessel. In certain embodiments, at least three engagement mechanisms abut a wall of the vessel. In certain embodiments, a plurality of engagement mechanisms abut the vessel. In certain embodiments, a plurality of engagement mechanisms abut a wall of the vessel. In certain embodiments, at least two engagement mechanisms are surface mounted to the vessel. In certain embodiments, at least two engagement mechanisms are surface mounted to a wall of the vessel. In certain embodiments, at least three engagement mechanisms are surface mounted to the vessel. In certain embodiments, at least three engagement mechanisms are surface mounted to a wall of the vessel. In certain embodiments, a plurality of engagement mechanisms are surface mounted to the vessel. In certain embodiments, a plurality of engagement mechanisms are surface mounted to a wall of the vessel.

In certain embodiments, the at least two engagement mechanisms are configured to engage at least two containers. In certain embodiments, the at least three engagement mechanisms are configured to engage at least three of the containers.

In certain embodiments, the vessel further comprising an exit port. In certain embodiments, the exit port abuts the vessel. In certain embodiments, the exit port abuts a wall of the vessel. In certain embodiments, the exit port is flush mounted to the vessel. In certain embodiments, the exit port is flush mounted to the vessel. In certain embodiments, the exit port is flush mounted to a wall of the vessel. In certain embodiments, the exit port is surface mounted to the vessel. In certain embodiments, the exit port is surface mounted to a wall of the vessel. In certain embodiments, the at least two containers are selected from the group consisting of a bottle, a bag, a syringe, a vial, a pre-loaded vial, a ready-to-assemble vial, a cartridge, and combinations thereof.

In certain embodiments, the at least two containers are designed to allow a user to double-check the identity of the beneficial substance and/or medicament housed in the containers by comprising an element selected from a group consisting of enlarged fonts, color coding, raised bumps, or protuberant.

In certain embodiments, the at least three containers are designed to allow a user to double-check the amount of the beneficial substance housed in the containers by comprising an element selected from a group consisting of enlarged fonts, color coding, raised bumps, or protuberant.

In certain embodiments, the system provides for the customization of a final amount of the beneficial substance and/or medicament that is assembled without the need to visually measure amounts of beneficial substance.

In certain embodiments, the system provides for customization of the final amount of the beneficial substance and/or medicament that is assembled without the need to manually manipulate the beneficial substance housed in the containers.

In certain embodiments, the system is modular and wherein amounts of different medicaments and/or beneficial substances are provided to the user via similar and/or different containers. In certain embodiments, the system is modular and wherein different amounts of medicaments and/or beneficial substances are provided to the user via similar and/or different containers.

In certain embodiments, the containers have different shapes and sizes and are configured to attach to the at least two entry ports of the vessel.

In certain embodiments, the at least two containers house a beneficial substance and/or medicament selected from a group consisting of a drug from Table 1 and/or Table 2 or a therapeutically equivalent formulation thereof.

In certain embodiments, at least one of the at least two containers house a beneficial substance and/or medicament in a non-standard amount. In certain embodiments, at least one of the at least two containers house a beneficial substance and/or medicament in an amount less than a standard amount.

In certain embodiments, at least one of the containers houses a beneficial substance and/or medicament in a subtherapeutic amount. In certain embodiments, at least two of the containers houses a beneficial substance and/or medicament in a subtherapeutic amount. In certain embodiments, at least three containers houses a beneficial substance and/or medicament in a subtherapeutic amount.

In certain embodiments, at least one of the containers houses a beneficial substance and/or medicament in a subtherapeutic amount for a typical patient. In certain embodiments, at least two of the containers houses a beneficial substance and/or medicament in a subtherapeutic amount for a typical patient. In certain embodiments, at least three containers houses a beneficial substance and/or medicament in a subtherapeutic amount for a typical patient. In certain embodiments, at least one of the containers houses a beneficial substance and/or medicament in an ineffective amount for a typical patient. In certain embodiments, at least two of the containers houses a beneficial substance and/or medicament in an ineffective amount for a typical patient. In certain embodiments, at least three containers houses a beneficial substance and/or medicament in an ineffective amount for a typical patient.

In certain embodiments, the at least one container is selected from a group consisting of two containers, three containers, four containers and five containers. In certain embodiments, the at least one container is a plurality of containers.

In certain embodiments, the at least two entry ports is selected from a group consisting of three entry ports, four entry ports, five entry ports, and six entry ports. In certain embodiments, the at least two entry ports are a plurality of entry ports. In one or more embodiments, the vessel has at least two exit ports. In one or more embodiments, the vessel has at least three exit ports. In one or more embodiments, the vessel has a plurality of exit ports.

In one or more embodiments, the plurality of exit ports abut the vessel. In one or more embodiments, the plurality of exit ports abut a wall of the vessel. In one or more embodiments, the plurality of exit ports abut at least two walls of the vessel. In one or more embodiments, the plurality of exit ports are flush mounted to the vessel. In one or more embodiments, the plurality of exit ports are flush mounted to a wall of the vessel. In one or more embodiments, the plurality of exit ports are surface mounted to the vessel. In one or more embodiments, the plurality of exit ports are surface mounted to a wall of the vessel. In one or more embodiments, the at least one container has at least two exit ports. In one or more embodiments, the at least one container has at least three exit ports. In one or more embodiments, the at least one container has at least four exit ports. In one or more embodiments, the at least one container has a plurality of exit ports. In one or more embodiments, the at least one container has at least two exit ports on the same wall of the at least one container. In one or more embodiments, at least two containers each have at least two exit ports. In one or more embodiments, the at least two containers each have a plurality of exit ports. In one or more embodiments, at least two containers each have at least two entry ports and at least two exit ports. In one or more embodiments, at least three containers each have at least two entry ports and at least two exit ports. In one or more embodiments, at least two containers each have a plurality of entry ports and a plurality of exit ports. In one or more embodiments, at least three containers each have a plurality of entry ports and a plurality of exit ports.

In certain embodiments, the vessel is selected from a group consisting of a bag, a bottle, or a syringe. In certain embodiments, the vessel is a cartridge.

In certain embodiments, the vessel includes a rigid surface around the at least two entry ports of the vessel. In certain embodiments, the vessel includes a rigid surface around the at least two exit ports. In certain embodiments, a surface of the vessel that supports the at least two entry ports has a greater rigidity than another surface of the vessel. In certain embodiments, the rigid surface around the at least two entry ports is of a sufficient strength to provide for a plurality of containers to engage and remain engaged to a side wall of the vessel. In certain embodiments, the rigid surface around the at least two entry ports is of a sufficient strength to provide for a plurality of containers to engage and remain engaged to a top wall of the vessel. In certain embodiments, the rigid surface around the at least two entry ports is of a sufficient strength to provide for a plurality of containers to engage and remain engaged to a bottom wall of the vessel.

In certain embodiments, the at least one exit port of the vessel is connected to an infusion line, filter, or needle.

In certain embodiments, one of the at least two entry ports is located on a surface of the vessel and another one of the at least two entry ports is located on a different surface of the vessel. In certain embodiments, the at least two entry ports are oriented randomly on one or more surfaces of the vessel.

In certain embodiments, the at least one container is selected from a group consisting of a vial, an ampule, a capsule, a cartridge, a pre-loaded vial, a pre-loaded ampule, a pre-loaded capsule, or a pre-loaded ampule.

In certain embodiments, the at least one container has an expulsion member. In certain embodiments, the expulsion member is a plunger. In certain embodiments, the at least one container is a syringe. In certain embodiments, the at least one container is a plurality of containers, wherein the plurality of containers are syringes. In certain embodiments, the at least one container is a plurality of containers, wherein the plurality of containers have an expulsion member. In certain embodiments, the at least one container is a plurality of containers, wherein the plurality of containers each have an expulsion member.

In certain embodiments, the at least one container is designed to allow the user to double check the identity and/or at least one amount by comprising an element selected from a group consisting of enlarged font written doses, color coding, raised bumps or protuberant. In certain embodiments, the at least one container is designed to allow the user to double check the identity and/or at least one amount by comprising a scanning element. In certain embodiments, the scanning element is a bar code.

In certain embodiments, at least one of the two entry ports includes a decontamination interface and/or decontamination device. In one or more embodiments, the vessel further comprises an exit port. In one or more embodiments, the exit port includes a decontamination interface and/or decontamination device.

In certain embodiments, the decontamination interface comprises: a first connection interface attached to one of the at least two entry ports of the vessel; and a second connection interface attached to the at least one container, wherein said first connection interface and said second connection interface are configured to allow for an engagement between said one of the at least two entry ports of the vessel and the at least one container, and wherein said first and second connection interfaces are further configured to externally displace from said engagement between said one of the at least two entry ports and said at least one container while a hermetically sealed connection is maintained between said first vessel and said second vessel.

In certain embodiments, the decontamination interface comprises: a first connection interface configured to be coupled to one of the at least two entry ports of the vessel; and a second connection interface configured to be coupled to the at least one container; wherein the first and second connection interfaces are configured to engage with each other and entrap contaminants, and wherein the first connection interface and the second connection interface, following said engagement, are configured to internally displace within the at least two entry ports or said at least one container, while allowing for a contaminant-free fluid passageway and hermetically sealed engagement of the vessel and the at least one container.

In certain embodiments, the decontamination interface comprises: a sliding mechanism positioned on one of the at least two entry ports of the vessel or on the at least one container, the sliding mechanism configured to allow traveling there along of the at least one container, such that the at least one container may move from a first position to a second position; and a wiping member disposed on one of the at least two entry ports of the vessel or the at least one container, said wiping member is configured to remove contaminants from a surface of one of the at least two entry ports of the vessel or the at least one container at about the time of said movement of the at least one container from said first position to said second position. In one or more embodiment, or optionally, the at least one container moves between a plurality of positions on or within the decontamination interface or decontamination device. In one or more embodiments, the wiping member is disposed within a housing of the vessel and/or the at least one container.

In certain embodiments, the decontamination interface comprises: a housing; a wiping member disposed within the housing, wherein the wiping member is configured to move within the housing; and wherein the wiping member decontaminates a surface of at least one of the at least two entry ports of the vessel or a surface of the at least one container.

In certain embodiment, the vessel having a plurality of decontamination devices.

In certain embodiments, the container includes one or more decontamination interfaces.

In certain embodiments, the decontamination interface includes a displaceable plate having a first plate part and a second plate part, and wherein the system is configured to allow: sealing the first container or device with the first plate part; sealing the second container or device with the second plate part; purging air at the interface while moving the displaceable plate.

In certain embodiments, the decontamination interface and/or device is selected from a group consisting of external displacement, internal displacement, moveable wiper, static wiper.

In certain embodiments, the decontamination interface abuts a wall of the container. In certain embodiments, the decontamination interface is flush mounted to the container. In certain embodiments, the decontamination interface is surface mounted to the container. In certain embodiments, the decontamination device abuts a wall of the container. In certain embodiments, the decontamination device is flush mounted to a wall of the container. In certain embodiments, the decontamination device is surface mounted to a wall of the container.

In certain embodiments, the at least one amount is selected from a group consisting of a drug from Table 1 or a therapeutically equivalent formulation/salt thereof. In certain embodiments, the at least one amount is selected from a group consisting of a drug from Table 2 or a therapeutically equivalent formulation/salt thereof.

In certain embodiments, the amount is in a non-standard amount or an amount not typically provided in a commercially packaged container.

In certain embodiments, the at least one container includes an amount of a drug or a therapeutically equivalent formulation thereof in an amount less than 10% of a drug set forth in Table 1 Column A or Table 2 Column A, less than 20% of a drug set forth in Table 1 Column A or Table 2 Column A, less than 30% of a drug set forth in Table 1 Column A or Table 2 Column A, less than 40% of a drug set forth in Table I Column A or Table 2 Column A, or less than 50% of a drug set forth in Table 1 Column A or Table 2 Column A.

In certain embodiments, a second container includes an amount of a drug or a therapeutically equivalent formulation thereof in an amount less than 10% of a drug set forth in Table 1 Column A or Table 2 Column A, less than 20% of a drug set forth in Table 1 Column A or Table 2 Column A, less than 30% of a drug set forth in Table I Column A or Table 2 Column A, less than 40% of a drug set forth in Table 1 Column A or Table 2 Column A, or less than 50% of a drug set forth in Table I Column A or Table 2 Column A.

In certain embodiments, a third container includes an amount of a drug or a therapeutically equivalent formulation thereof in an amount less than 10% of a drug set forth in Table 1 Column A or Table 2 Column A, less than 20% of a drug set forth in Table 1 Column A or Table 2 Column A, less than 30% of a drug set forth in Table I Column A or Table 2 Column A, less than 40% of a drug set forth in Table 1 Column A or Table 2 Column A, or less than 50% of a drug set forth in Table I Column A or Table 2 Column A.

In certain embodiments, a fourth container includes an amount of a drug or a therapeutically equivalent formulation thereof in an amount less than 10% of a drug set forth in Table 1 Column A or Table 2 Column A, less than 20% of a drug set forth in Table 1 Column A or Table 2 Column A, less than 30% of a drug set forth in Table 1 Column A or Table 2 Column A, less than 40% of a drug set forth in Table I Column A or Table 2 Column A, or less than 50% of a drug set forth in Table 1 Column A or Table 2 Column A.

In certain embodiments, a fifth container includes an amount of the least one amount or a therapeutically equivalent formulation thereof in an amount less than 10% of a drug set forth in Table 1 Column A or Table 2 Column A, less than 20% of a drug set forth in Table 1 Column A or Table 2 Column A, less than 30% of a drug set forth in Table 1 Column A or Table 2 Column A, less than 40% of a drug set forth in Table 1 Column A or Table 2 Column A, or less than 50% of a drug set forth in Table 1 Column A or Table 2 Column A.

In certain embodiments, another container includes an amount of the at least one dose/amount or a therapeutically equivalent formulation thereof in an amount less than or greater than a standard prepackaged commercially available amount as set forth in Tables 1 and 2.

In certain embodiments, the system provides for customization of the amount within the vessel.

In certain embodiments, the system is modular and wherein amounts of different medicaments are provided to the user via different containers.

In certain embodiments, the containers have different shapes and sizes and are configured to attach to the at least two entry ports of the vessel.

In another aspect, the present invention provides a method for adding at least one amount of a medicament and/or a beneficial substance to a vessel, the method comprising: providing a vessel including at least two entry ports and at least one exit port; providing at least one container including at least one amount of a medicament; connecting the at least one container to one of the at least two entry ports of the vessel; wherein the at least one container is directly received and engaged by one of the at least two entry ports of the vessel; and transferring the at least one amount of a medicament in the at least one container to the vessel.

In certain embodiments, the at least one container is selected from a group consisting of two containers, three containers, four containers and five containers. In certain embodiments, the method further comprises connecting two of the containers to the vessel and transferring the contents of the two containers into the vessel. In certain embodiments, the method further comprises connecting three of the containers to the vessel and transferring the contents of the three containers into the vessel. In certain embodiments, the method further comprises connecting four containers to the vessel and transferring the contents of the four containers into the vessel. In certain embodiments, the method further comprises connecting five containers to the vessel and transferring the contents of the five containers into the vessel. In certain embodiments, the method further comprises connecting six or more containers to the vessel and transferring the contents of the six or more containers into the vessel.

In certain embodiments, the at least one amount is in a non-standard amount. In certain embodiments, the at least one container is a plurality of containers, wherein each of the plurality of containers houses a medicament and/or beneficial substance in a non-standard amount.

In certain embodiments, the at least one container is two containers, wherein each of the two containers houses a medicament and/or beneficial substance in a non-standard amount. In certain embodiments, the at least one container is three containers, wherein each of the three containers houses a medicament and/or beneficial substance in a non-standard amount. In certain embodiments, the at least one container is four containers, wherein each of the four containers houses a medicament and/or a beneficial substance in a non-standard amount. In certain embodiments, the at least one container is five containers, wherein each of the five containers houses a medicament and/or beneficial substance in a non-standard amount.

In certain embodiments, the at least one container is a plurality of containers, wherein at least one container houses a medicament and/or beneficial substance in a standard amount and wherein at least one container houses a medicament and/or a beneficial substance in a non-standard amount.

In certain embodiments, at least two of the containers may be stacked one upon the other. In certain embodiments, at least three of the containers may be stacked one upon the other. In certain embodiments, at least four of the containers may be stacked one upon the other. In certain embodiments, at least five of the containers may be stacked one upon the other. In certain embodiments, a plurality of the containers may be stacked one upon the other. In certain embodiments, the stacking of the containers is in a vertical configuration, wherein a force of gravity pulls the contents of the containers downward towards an exit port.

In certain embodiments, the stacking of the containers may be in a vertical and horizontal configuration. In certain embodiments, the connection of a plurality of containers to one another may be in a randomized configuration.

In certain embodiments, the vessel is selected from a group consisting of a bag, a bottle, or a syringe. In certain embodiments, the vessel may be a container. In certain embodiments, the vessel may be a container similar to the at least one container.

In certain embodiments, the at least one exit port of the vessel is connected to an infusion line, filter, connector, decontamination device or needle.

In certain embodiments, the at least one container is selected from a group consisting of a vial, an ampule, a capsule, a cartridge, a pre-loaded vial, a pre-loaded ampule, a pre-loaded capsule, or a pre-loaded ampule.

In certain embodiments, at least one of the two entry ports includes a decontamination interface. In one or more embodiments, at least two entry ports are coupled to a decontamination device or decontamination interface.

In certain embodiments, the connection between the vessel and the at least one container includes a decontamination interface, the decontamination interface comprises: a first connection interface attached to one of the at least two entry ports of the vessel; and a second connection interface attached to the at least one container, wherein said first connection interface and said second connection interface are directly received and engaged between said one of the at least two entry ports of the vessel and the at least one container, and wherein said first and second connection interfaces are externally displaced from said engagement between said one of the at least two entry ports and said at least one container while a hermetically sealed connection is maintained between said one of the at least two entry ports and said at least one container.

In certain embodiments, the decontamination interface comprises: a first connection interface is coupled to one of the at least two entry ports of the vessel; and a second connection interface is coupled to the at least one container; wherein the first and second connection interfaces are engaged with each other and entrap contaminants, and wherein the first connection interface and the second connection interface, following said engagement, are internally displaced within the at least two entry ports or said second at least one container, while providing for a contaminant-free fluid passageway and hermetically sealed engagement of the vessel and the at least one container.

In certain embodiments, the at least one container is selected from a group consisting of two containers, three containers, four containers and five containers.

In certain embodiments, the at least two entry ports is selected from a group consisting of three entry ports, four entry ports, five entry ports, and six entry ports.

In another aspect, the present invention is directed to a method of decontaminating a modular assembly system for beneficial substances, comprising: providing a vessel having a plurality of decontamination devices; providing a plurality of containers; and coupling the plurality of containers to the plurality of decontamination devices.

In certain embodiments, the method comprises decontaminating the surfaces of a plurality of containers. In certain embodiments, the plurality of containers are attached to the plurality of decontamination devices sequentially.

In certain embodiments, the plurality of containers are attached to the plurality of decontamination devices randomly.

In certain embodiments, the surfaces of the containers are decontaminated sequentially. In certain embodiments, the surfaces of the containers are decontaminated in a random order.

In certain embodiments, the method further comprises moving a wiping member across a plurality of housings of the decontamination devices. In certain embodiments, the method further comprises moving a plurality of the containers between a plurality of compartments of the decontamination devices.

In another aspect, the present invention is directed to a syringe comprising: at least two entry ports and at least one exit port, the syringe configured to receive at least one container including at least one amount of a medicament; wherein the at least one container is configured to be directly received and engaged by one of the at least two entry ports of the syringe, wherein upon connection of the at least one container to the one of the at least two entry ports of the syringe, the at least one amount in the at least one container is transferred into the syringe.

In another aspect, the present invention provides a system for assembling a beneficial substance, the system comprising: a vessel having at least two entry ports configured to directly receive and engage at least two containers; at least two containers configured to be directly received and engaged by the at least two entry ports of the vessel, wherein the at least two containers house a beneficial substance; wherein upon engagement of the at least two containers to the at least two entry ports of the vessel, the beneficial substance housed in the at least two containers is transferred into the vessel.

In certain embodiments, the at least two entry ports of the vessel abut a wall of the vessel. In certain embodiments, the at least two entry ports of the vessel are flush mounted or are surface mounted to a wall of the vessel. In certain embodiments, the vessel is a bag, bottle, syringe or combinations thereof.

In certain embodiments, the vessel has at least three entry ports. In certain embodiments, the engagement between the at least two entry ports of the vessel and the at least two containers is selected from a thread-luer mechanism, a ratchet teeth mechanism, an adhesive mechanism, a slide-on mechanism and a snap-on mechanism.

In certain embodiments, the vessel includes a rigid surface around the at least two entry ports of the vessel.

In certain embodiments, a surface of the vessel that supports the at least two entry ports has a greater rigidity than another surface of the vessel.

In certain embodiments, the at least two containers have a decontamination device. In certain embodiments, the decontamination device is selected from the group consisting of external displacement, internal displacement, static wiper, and moveable wiper decontamination device. In certain embodiments, the vessel has at least two decontamination devices configured to establish a contaminant-free engagement between the at least two entry ports of the vessel and the at least two containers. In certain embodiments, the at least two decontamination devices are attached to the at least two entry ports of the vessel. In certain embodiments, the at least two decontamination devices abut a wall of the vessel. In certain embodiments, the at least two decontamination devices are flush mounted or are surface mounted to a wall of the vessel.

In certain embodiments, the vessel further comprises an exit port. In certain embodiments, the exit port of the vessel is connected to an infusion line, a filter, or a needle. In certain embodiments, the exit port is connected to a decontamination device.

In certain embodiments, at least one container of the at least two containers houses an amount of a beneficial substance or a therapeutically equivalent formulation thereof in an amount less than about 5% of a drug set forth in Table 1 Column A or Table 2 Column A, less than about 10% of a drug set forth in Table 1 Column A or Table 2 Column A, less than about 15% of a drug set forth in Table 1 Column A or Table 2 Column A, less than about 20% of a drug set forth in Table 1 Column A or Table 2 Column A, or less than about 25% of a drug set forth in Table 1 Column A or Table 2 Column A.

In certain embodiments, at least one container of the at least two containers houses an amount of a beneficial substance or a therapeutically equivalent formulation thereof in an amount less than about 30% of a drug set forth in Table 1 Column A or Table 2 Column A, less than about 35% of a drug set forth in Table 1 Column A or Table 2 Column A, less than about 40% of a drug set forth in Table 1 Column A or Table 2 Column A, or less than about 50% of a drug set forth in Table 1 Column A or Table 2 Column A.

In certain embodiments, a second container of the at least two containers houses an amount of a beneficial substance or a therapeutically equivalent formulation thereof in an amount less than about 5% of a drug set forth in Table 1 Column A or Table 2 Column A, less than about 10% of a drug set forth in Table 1 Column A or Table 2 Column A, less than about 15% of a drug set forth in Table 1 Column A or Table 2 Column A, less than about 20% of a drug set forth in Table 1 Column A or Table 2 Column A, or less than about 25% of a drug set forth in Table 1 Column A or Table 2 Column A.

In certain embodiments, a second container of the at least two containers houses an amount of a beneficial substance or a therapeutically equivalent formulation thereof in an amount less than about 30% of a drug set forth in Table 1 Column A or Table 2 Column A, less than about 35% of a drug set forth in Table 1 Column A or Table 2 Column A, less than about 40% of a drug set forth in Table 1 Column A or Table 2 Column A, or less than about 50% of a drug set forth in Table 1 Column A or Table 2 Column A.

In another aspect, the present invention provides a system for the assembly of a beneficial substance, the system comprising: a vessel having at least two entry ports abutting the vessel, the at least two entry ports configured to receive at least two containers; at least two containers configured to be received by the at least two entry ports of the vessel, wherein the at least two containers are configured to be directly received by the at least two entry ports of the vessel.

In another aspect, the present invention provides a system for the assembly of a beneficial substance, the system comprising: a bag having at least two entry ports abutting the bag, the at least two entry ports configured to receive at least two containers; at least two containers configured to be received by the at least two entry ports of the bag, wherein the at least two containers are configured to be directly received by the at least two entry ports of the bag.

In another aspect, the present invention provides a first container for the assembly of a beneficial substance, the container having at least two entry ports abutting the first container, the at least two entry ports configured to receive at least two containers.

In another aspect, the present invention provides a system comprising a plurality of containers each having at least one entry port and at least one exit port, wherein the plurality of the containers are configured to engage one another in a sequential and/or randomized configuration, wherein upon and/or after engagement of the plurality of the containers, the plurality of the containers are in fluidic communication with one another.

In certain embodiments, the engagement between the plurality of the containers is an airtight engagement. In certain embodiments, the engagement between the plurality of the containers is a hermetic engagement. In certain embodiments, the engagement between the plurality of the containers is irreversible and/or permanent. In certain embodiments, the plurality of containers have a medicament, and/or a beneficial substance housed in the containers. In certain embodiments, the containers are configured to engage each other in a stacking configuration. In certain embodiments, the stacking configuration is a vertical or horizontal stacking configuration.

In certain embodiments, the engagement and fluidic communication between the plurality of the containers provides for the medicament and/or beneficial substance to flow through at least one of the containers. In certain embodiments, the engagement and fluidic communication between the plurality of the containers provides for the medicament and/or beneficial substance to flow through at least two of the containers. In certain embodiments, the engagement and fluidic communication between the plurality of the containers provides for the medicament and/or beneficial substance to flow through a plurality of the containers. In certain embodiments, a force of gravity pulls the medicament and/or beneficial substance downward through a plurality of the containers.

In certain embodiments, the system of the plurality of the containers provides for the preparation and/or administration of a customized and/or individualized amount of a medicament and/or beneficial substance.

In certain embodiments, the system of the plurality of the containers is modular. In certain embodiments, the system provides for the modular assembly of beneficial substances without manual manipulation of the beneficial substances.

In another aspect, the present invention provides a bag for the assembly of a beneficial substance, the bag having at least two entry ports abutting the bag, the at least two entry ports configured to receive at least two containers.

In certain embodiments, the bag has at least two entry ports flush mounted to the bag. In certain embodiments, the bag has at least two entry ports surface mounted to the bag. In certain embodiments, the at least two entry ports of the bag are made of a rigid material. In certain embodiments, the at least two entry ports of the bag are made of a material of sufficient strength to provide for the engagement of the at least two containers to a side wall of the bag. In certain embodiments, the at least two entry ports are a plurality of entry ports made of a material of sufficient strength to provide for the engagement and maintenance of the engagement of the plurality of containers to a side wall of the bag. In certain embodiments, the at least two entry ports of the bag are made of a glass material. In certain embodiments, the at least two entry ports of the bag are made of a metal material. In certain embodiments, the at least two entry ports of the bag are made of a plastic material. In certain embodiments, the at least two entry ports are made of a rigid plastic material.

In certain embodiments, the at least two entry ports have a thickness of at least one-eighth of an inch. In certain embodiments, the at least two entry ports have a thickness of at least one-quarter of an inch.

In certain embodiments, the at least two entry ports have a thickness of at least half an inch. In certain embodiments, the at least two entry ports of the bag have a compartment that has a pressure less than environmental or atmospheric pressure.

In certain embodiments, the at least two entry ports of the bag have a compartment that has a pressure less than the pressure of the at least two containers. In certain embodiments, the at least two entry ports of the bag have a compartment that houses a sterilizing and/or disinfecting substance. In certain embodiments, the at least two entry ports of the bag are sealed and/or covered. In certain embodiments, the seal and/or cover is a displaceable or a frangible seal and/or cover.

In another aspect, the present invention provides a system for the assembly of a beneficial substance, the system comprising:

a vessel having at least two entry ports flush mounted to the vessel, the at least two entry ports configured to receive at least two containers; at least two containers configured to be received by the at least two entry ports of the vessel, wherein the at least two containers are configured to be directly received by the at least two entry ports of the vessel.

In another aspect, the present invention provides a system for the assembly of a beneficial substance, the system comprising: a bag having at least two entry ports surface mounted to the bag, the at least two entry ports configured to receive at least two containers; at least two containers configured to be received by the at least two entry ports of the bag; wherein the at least two containers are configured to be directly received by the at least two entry ports of the bag.

In another aspect, the present invention provides a system for the assembly of a beneficial substance, the system comprising: a bag having at least two entry ports, the at least two entry ports configured to receive at least two connectors; at least two connectors configured to be received by the at least two entry ports of the bag; and at least two containers configured to be received by the at least two connectors; wherein the at least two connectors are received and engaged to the at least two entry ports of the bag and wherein the at least two containers are received and engaged to the at least two connectors a fluidic communication is established between the at least two containers and the at least two entry ports of the bag.

In certain embodiments, the at least two connectors have at least one decontamination device disposed on the connector. In certain embodiments, the at least two connectors have at least two decontamination devices disposed on the connector.

In certain embodiments, the at least two connectors have at least one displaceable interface or surface. In certain embodiments, the at least two connectors have at least two displaceable interfaces or surfaces. In certain embodiments, the at least two containers have a displaceable interface or surface. In certain embodiments, the at least two entry ports have a displaceable interface or surface. In certain embodiments, the at least two connectors have a decontamination device attached to the connectors. In certain embodiments, the at least two entry ports have a decontamination device attached to the entry ports. In certain embodiments, the at least two containers have a decontamination device attached to the entry ports. In certain embodiments, the at least two connectors each have a conduit disposed within the connector for providing a fluidic passageway between the at least two containers and the bag.

In another aspect, the present invention provides a vessel for the assembly of a beneficial substance, the vessel having at least two entry ports flush mounted to the vessel, the at least two entry ports configured to receive at least two containers and/or connectors.

In another aspect, the present invention provides a bag for the assembly of a beneficial substance, the bag having at least two entry ports flush mounted to the bag, the at least two entry ports configured to receive at least two containers and/or connectors. In another aspect the present invention provides a vessel for the assembly of a beneficial substance, the vessel having at least two entry ports surface mounted to the vessel, the at least two entry ports configured to receive at least two containers and/or at least two connectors. In another aspect the present invention provides a bag for the assembly of a beneficial substance, the bag having at least two entry ports surface mounted to the bag, the at least two entry ports configured to receive at least two containers and/or at least two connectors.

In another aspect, the present invention provides a system for the assembly of a beneficial substance, the system comprising: a syringe having at least two entry ports, the at least two entry ports configured to receive at least two containers; and at least two containers configured to be received by the at least two entry ports of the syringe.

In certain embodiments, the at least two entry ports abut the syringe. In certain embodiments, the at least two entry ports are flush mounted to the syringe. In certain embodiments, the at least two entry ports are surface mounted to the syringe. In certain embodiments, at least one of the at least two containers house a beneficial substance and/or medicament in a non-standard amount. In certain embodiments, the at least two containers house a beneficial substance and/or medicament in a non-standard amount.

In another aspect, the present invention provides a system for the assembly of a beneficial substance, the system comprising: a syringe having at least two entry ports flush mounted to the syringe, the at least two entry ports configured to receive at least two containers; and at least two containers configured to be received by the at least two entry ports of the syringe.

In another aspect, the present invention provides a syringe for the assembly of a beneficial substance, the syringe having at least two entry ports abutting the syringe, the at least two entry ports configured to receive at least two containers.

In another aspect, the present invention provides a syringe for the assembly of a beneficial substance, the syringe having at least two entry ports flush mounted to the syringe, the at least two entry ports configured to receive at least two containers. In another aspect, the present invention provides a syringe for the assembly of a beneficial substance, the syringe having at least two entry ports surface mounted to the syringe, the at least two entry ports configured to receive at least two containers.

In another aspect, the present invention provides a syringe for the assembly of a beneficial substance, the syringe having at least one entry port and at least one exit port, the at least one entry port configured to receive at least one container. In another aspect, the present invention provides a syringe for the assembly of a beneficial substance, the syringe having at least one entry port, the at least one entry port configured to receive at least one container. In another aspect, the present invention provides a syringe for the assembly of a beneficial substance, the syringe having at least one entry port, the at least one entry port configured to directly receive at least one container. In another aspect, the present invention provides a syringe for the assembly of a beneficial substance, the syringe having at least two entry ports, the at least two entry ports configured to receive at least two containers. In another aspect, the present invention provides a syringe for the assembly of a beneficial substance, the syringe having at least two entry ports, the at least two entry ports configured to directly receive at least two containers. In another aspect, the present invention provides a syringe for the assembly of a beneficial substance, the syringe having at least one entry port and one exit port, the at least one entry port configured to receive a connector. In another aspect, the present invention provides a syringe for the assembly of a beneficial substance, the syringe having at least two entry ports, the at least two entry ports configured to receive at least two connectors.

In another aspect, the present invention provides a syringe having at least one decontamination device. In another aspect, the present invention provides a syringe with plurality of decontamination devices. In another aspect, the present invention provides a syringe with a plurality of entry ports and a plurality of decontamination devices.

In another aspect, the present invention provides a syringe with at least two decontamination devices covering at least two entry ports. In another aspect, the present invention provides a container with an expulsion member having at least two entry ports, the entry ports configured to engage at least two containers. In another aspect the present invention provides a container with an expulsion member having at least two entry ports, the entry ports configured to engage at least two connectors. In another aspect, the present invention provides a container having an expulsion member, at least two entry ports, and at least two displaceable interfaces and/or surfaces. In another aspect, the present invention provides a container having an expulsion member, at least two entry ports, and at least two decontamination devices. In another aspect, the present invention provides a container having a decontamination device on or attached to an exit port of the container.

In certain embodiments, the container is a syringe.

In another aspect, the present invention provides a syringe having a decontamination device on or attached to an exit port of the syringe.

In another aspect, the present invention provides a system for the assembly of a beneficial substance, comprising: a bag having at least one piercing member disposed within the bag and at least one entry ports, wherein the at least one entry port is configured to receive at least one container; and at least one container configured to be received by the at least one entry port of the bag.

In certain embodiments, the piercing member is disposed within a port of the bag. In certain embodiments, the piercing member is disposed within a chamber and/or compartment of the bag. In certain embodiments, the piercing member is concealed from ambient and/or environmental air. In certain embodiments, the piercing member is concealed from ambient and/or environmental air until about the time of piercing and/or actuation of the piercing member. In certain embodiments, the piercing member is a needle. In certain embodiments, the needle is a hollowed needle. In certain embodiments, the hollowed needle is configured to provide a fluidic passageway between the bag and the at least one container. In certain embodiments, the container is a vial. In certain embodiments, the container is a bottle. In certain embodiments, the container has an expulsion member.

In another aspect, the present invention provides a bag having at least one piercing member disposed within the bag and at least one entry port abutting the bag, wherein the at least one entry port is configured to receive at least one container; and at least one container configured to be received by the at least one entry port of the bag.

In certain embodiments, the piercing member is disposed within a port of the bag. In certain embodiments, the piercing member is disposed within a chamber and/or compartment of the bag. In certain embodiments, the piercing member is concealed from ambient and/or environmental air. In certain embodiments, the piercing member is concealed from ambient and/or environmental air until about the time of piercing and/or actuation of the piercing member. In certain embodiments, the piercing member is a needle. In certain embodiments, the needle is a hollowed needle. In certain embodiments, the hollowed needle is configured to provide a fluidic passageway between the bag and the at least one container.

In certain embodiments, the container is a vial. In certain embodiments, the container is a bottle. In certain embodiments, the container has an expulsion member.

In another aspect, the present invention provides a bag having at least one piercing member disposed within the bag and at least one entry port flush mounted to the bag, wherein the at least one entry port is configured to receive at least one container; and at least one container configured to be received by the at least one entry port of the bag.

In certain embodiments, the piercing member is disposed within a port of the bag. In certain embodiments, the piercing member is disposed within a chamber and/or compartment of the bag. In certain embodiments, the piercing member is concealed from ambient and/or environmental air. In certain embodiments, the piercing member is concealed from ambient and/or environmental air until about the time of piercing and/or actuation of the piercing member. In certain embodiments, the piercing member is a needle. In certain embodiments, the needle is a hollowed needle. In certain embodiments, the hollowed needle is configured to provide a fluidic passageway between the bag and the at least one container.

In certain embodiments, the container is a vial. In certain embodiments, the container is a bottle. In certain embodiments, the container has an expulsion member.

In another aspect, the present invention provides a bag having at least one piercing member disposed within the bag and at least one entry port, wherein the at least one entry port is configured to receive at least one container.

In another aspect, the present invention provides a bag having at least one piercing member disposed within the bag and at least one entry port abutting the bag, wherein the at least one entry port is configured to receive at least one container.

In another aspect, the present invention provides a bag having at least one piercing member disposed within the bag and at least one entry port surface mounted to the bag, wherein the at least one entry port is configured to receive at least one container.

In certain embodiments, the piercing member is disposed within a port of the bag. In certain embodiments, the piercing member is disposed within a chamber and/or compartment of the bag. In certain embodiments, the piercing member is concealed from ambient and/or environmental air. In certain embodiments, the piercing member is concealed from ambient and/or environmental air until about the time of piercing and/or actuation of the piercing member. In certain embodiments, the piercing member is a needle. In certain embodiments, the needle is a hollowed needle. In certain embodiments, the hollowed needle is configured to provide a fluidic passageway between the bag and the at least one container.

In certain embodiments, the container is a vial. In certain embodiments, the container is a bottle. In certain embodiments, the container has an expulsion member. In one or more embodiments, the piercing member is concealed from ambient air. In one or more embodiments, the piercing member is disposed within a port of the bag. In one or more embodiments, the piercing member is disposed within the main chamber of the bag. In one or more embodiments, the piercing member is disposed within a secondary chamber of the bag. In one or more embodiments, the bag further comprises a second piercing member disposed within the bag. In one or more embodiments, a third piercing member is disposed within the bag. In one or more embodiments, at least a fourth piercing member is disposed within the bag. In one or more embodiments, the bag has a plurality of piercing members disposed within the bag and concealed from ambient air. In one or more embodiments, the plurality of piercing members are disposed within a port of the bag. In one or more embodiments, the plurality of piercing members are disposed within the main chamber of the bag. In one or more embodiments, the plurality of piercing members are disposed within a secondary chamber of the bag.

In one or more embodiments, an actuator is in the bag and is configured to move the piercing member through a surface of the bag. In one or more embodiments, a plurality of actuators are configured to move the piercing members through a surface of the bag. In one or more embodiments, an unlocking mechanism is provided and is configured to unlock the at least one piercing member. In one or more embodiments, an unlocking mechanism is provided and is configured to unlock the at least two actuators thus allowing the actuators to move the at least two piercing members through a surface of the bag.

In one or more embodiments, an unlocking member is configured to unlock the at least one piercing member. In one or more embodiments, an unlocking member is configured to unlock the at least two actuators thus allowing the actuators to move the at least two piercing members through a surface of the bag.

In one or more embodiments, an unlocking member on bag or vial engages an unlocking mechanism on other bag or vial resulting in release of a piercing member/actuator/displaceable vessel surface or seal.

In one or more embodiments, fluidic communication cannot be established unless the unlocking member engages the unlocking mechanism. This safety feature provides for hermetic engagement first, then afterwards establishment of fluidic communication. This prevents toxic chemotherapeutic substance in a vial from prematurely being released/spilled into the environmental surroundings/ambient air.

In one or more embodiments, a plurality of piercing members are provided that are concealed from ambient air. In one or more embodiments, a plurality of piercing members are provided that are concealed from ambient air and a plurality of entry ports are configured to receive a plurality of containers. In one or more embodiments, the piercing members are needles. In one or more embodiments, the needles are hollowed needles. In one or more embodiments, the piercing members have at least one sharp surface. In one or more embodiments, the piercing member are located inside a vessel. In one or more embodiments, the piercing members are located inside the bag. In one or more embodiments, the piercing member are located within the ports of the bag. In one or more embodiments, the piercing members are located in a chamber of the bag. In one or more embodiments, the piercing members are located within a plurality of entry ports of the bag. In one or more embodiments, the piercing member is located within at least one entry port of the bag.

In one or more embodiments, bags/containers may have locking mechanisms that lock another container to the bag/first container. In one or more embodiments fixed locking and/or irreversible locking occurs. In one or more embodiments, first engagement and locking occurs, then hermetic seal established, then fluidic communication. In one or more embodiments, first a hermetic seal is established between the bag and the containers, then locking occurs, then fluid communication between the bag and the containers.

In one or more embodiments, bags/containers may have “unlocking” mechanisms/members that “unlock the piercing member” on the other container, so that the piercing members don't “prematurely” pierce the vessel surfaces.

In one or more embodiments, bags/containers may have unlocking members that unlock an “actuator” that moves the piercing members that are disposed in the bag/container.

In one or more embodiments, the piercing members are concealed from ambient air. In one or more embodiments, other fluidic communication mechanisms are contemplated in addition to the piercing members. In one or more embodiments, a displaceable container surface that is “unlocked” at about the time of engagement of containers. In one or more embodiments, the displaceable container is unlocked after engagement of containers. In one or more embodiments, the container surface may be unlocked by an unlocking member located on another container. In one or more embodiments, the container surface then displaces allowing for fluidic communication between containers.

In another aspect, the present invention provides a modular dosing system for adding at least amount and/or at least one dose of a medicament to a vessel or container, the system comprising: at least two containers, at least one of the at least two containers including an amount and/or dose of a medicament and/or beneficial substance; wherein each of the at least two containers are configured to be connected to at least one another one of the at least two containers, wherein upon connection of one of the at least two containers to another one of the at least two containers, the dose of medicament in one of the at least two containers is in fluid communication with the other one of the at least two containers.

In certain embodiments, at least one of the at least two containers has a decontamination interface which operates as a dual entry and exit port. In certain embodiments, at least one of the at least two containers has a decontamination interface which operates as the exit port into a device selected from the following group of an infusion line, a filter, a manifold, a connector, or a needle. In certain embodiments, the force of gravity pulls the amount of medicament into an infusion line that operates as the exit port. In certain embodiments, at least one of the at least two containers are attached to the other container having at least one of the entry ports in a vertical or horizontal manner on any side of the container.

In certain embodiments, the system is selected from a group consisting of two containers, three containers, four containers or five containers. In certain embodiments, the at least two containers are selected from a group consisting of a vial, an ampule, a capsule, a cartridge, a pre-loaded vial, a pre-loaded ampule, a pre-loaded capsule, or a pre-loaded ampule. In certain embodiments, the at least two containers are designed to allow the user to double check the dosage regimen by comprising an element selected from a group consisting of enlarged font written doses, color coding, raised bumps or protuberant located on the at least two containers.

In certain embodiments, the decontamination interface comprises: a first connection interface attached to one of the at least two entry ports of the vessel; and a second connection interface attached to the at least one container, wherein said first connection interface and said second connection interface are configured to allow for an engagement between said one of the at least two entry ports of the vessel and the at least one container, and wherein said first and second connection interfaces are further configured to externally displace from said engagement between said one of the at least two entry ports and said at least one container while a hermetically sealed connection is maintained between said first vessel and said second vessel.

In certain embodiments, the decontamination interface comprises: a first connection interface configured to be coupled to one of the at least two entry ports of the vessel; and a second connection interface configured to be coupled to the at least one container; wherein the first and second connection interfaces are configured to engage with each other and entrap contaminants, and wherein the first connection interface and the second connection interface, following said engagement, are configured to internally displace within the at least two entry ports or said at least one container, while allowing for a contaminant-free fluid passageway and hermetically sealed engagement of the vessel and the at least one container.

In certain embodiments, the dosing regimen is selected from a group consisting of various drugs set forth in Table 1 and/or Table 2, or a therapeutically equivalent formulation/salt thereof.

In certain embodiments, the dosing regimen is in a non-standard amount or an amount not provided in a commercially prepackaged container.

In another aspect, the present invention provides a method for adding one amount of a medicament to a modular dosing system, the method comprising: providing at least one container with at least one entry port and at least one exit port; providing at least one other container with at least one other exit port; connecting the at least one container with at least one entry port and at least one exit port to the at least one other container with at least one other exit port, transferring the beneficial substance or medicament from the at least one other container with at least one other exit port into the at least one container with at least one entry port and at least one exit port.

In certain embodiments, the connecting of the at least one container with at least one entry port and at least one exit port to the at least one other container with at least one other exit port, can be connected in any of the following ways or combination thereof: horizontal, vertical, lateral, normal, diagonal, longitudinal, linear, three-dimensional or other orientations.

In another aspect, the present invention provides a system for the assembly of a beneficial substance, the system comprising: a first container having at least one entry port configured to engage a second container; a second container having at least one entry ports and at least one exit port, wherein the at least one entry port is configured to engage a third container and the at least one exit port is configured to engage the first container; at least a third container having at least one exit port; wherein upon engagement of the first container to the second container, and upon engagement of the second container to the third container, the contents of the third container flows into the second container and flows-through along with the contents of the second container into the first container. In one or more embodiments, the system for assembling the beneficial substance is modular by design. In one or more embodiments, a fourth container, a fifth container, or six or more containers may be provided by the system allowing a user of the system to modularly assemble any final amount of a beneficial substance and/or medicament.

In certain embodiments, the second container houses a beneficial substance, wherein the beneficial substance from the third container flows-through the second container and into the first container. In one or more embodiments, the modular system provides beneficial substances and/or medicaments in commercially packaged non-standard amounts. In one or more embodiments, commercially packaged means prepackaged. In one or more embodiments, commercially packaged means packaged by a drug/pharmaceutical manufacturer.

In another aspect, the present invention provides a system for displaying a dosing regimen or single amount of a medicament, the system comprising: a vessel including at least one entry port and at least one exit port; at least one other container including at least one dosing regimen, the amount of at least one dosing regimen being displayed upon an exterior surface of the at least one container; wherein the at least one container is configured to be received by one of the at least one entry ports of the vessel; and the exterior surface of the container is configured to allow the user to easily double check and identify the contents and amounts of the dosing regimen.

In certain embodiments, the connection of the at least one container to the vessel allows for a user to view the dosing regimen written upon the exterior surface of the at least one container, so that the user is able to quickly identify the dosing regimen provided to the patient.

In certain embodiments, the displayed amounts are in non-standard amounts.

In certain embodiments, the displayed amounts are in standard amounts. In certain embodiments, the displayed amounts are in non-standard and standard amounts. In certain embodiments, the displayed amounts are in different colors to indicate different types of amounts and rapidly identify the contents of the at least one container. In certain embodiments, the displayed amounts are written on an exterior surface of the at least one container. In certain embodiments, the at least one container includes at least one protrusion including the displayed amounts.

In certain embodiments, the exterior surface contains at least one raised bump or protuberance to indicate different types of amounts and rapidly identify the contents of the at least one container. In certain embodiments, the exterior surface display contains at least one geometric shape to indicate different types of amounts and rapidly identify the contents of the at least one container. In certain embodiments, the exterior surface display reflects the said at least one container's total volume. In certain embodiments, the exterior surface display of the at least one container is demarcated with at least one line such that the amount of the dosing regimen inside can be visually compared against the at least one demarcation line for an estimate of the total volume.

In certain embodiments, the exterior surface of the vessel is demarcated with at least one line such that the amount of the dosing regimen inside can be visually compared against the at least one demarcation line for an estimate of the total volume.

In other aspects, the present invention provides a method for double checking the dosage container contents in a dosing system, the method comprising the steps of: providing at least one container designed to be modular and part of a potential plurality of containers, hence the necessity for double checking dosage; providing an exterior surface of a container with an amount of medicament whose contents indicated by at least one item selected from the following group or a combination thereof: markings, letters, numbers, physical bumps and indentations, or colorings that are legible to the naked human eye.

In other aspects, the present invention provides a method for double checking the identity container contents in a dosing system, the method comprising the steps of: providing at least one container designed to be modular and part of a potential plurality of containers, hence the necessity for double checking identity; providing an exterior surface of a container with an amount of medicament whose contents indicated by at least one item selected from the following group or a combination thereof: markings, letters, numbers, physical bumps and indentations, or colorings that are legible to the naked human eye.

In certain embodiments, the contents indication is inverted.

In other aspects, the present invention provides a plurality of entry ports abutting a wall of a container. In certain embodiments a plurality of entry ports are flush mounted to a wall of a container. In certain embodiments, a plurality of entry ports are surface mounted to a wall of a container.

In certain embodiments, a plurality of engagement mechanisms abut a wall of the container, are surface mounted to the container or are flush mounted to the container. In certain embodiments, the engagement mechanisms are selected from a group consisting of threads, a luers, smart sites, ratchet teeth, etc.

In other aspects, the present invention provides a plurality of decontamination devices abutting, flush mounted, or surface mounted to a wall of a container.

In other aspects, the present invention provides a system for the modular assembly of a beneficial substance, comprising: a vessel having a plurality of openings configured to couple with a plurality of containers; a plurality of containers having a decontamination device, wherein the containers are configured to couple with the vessel.

In certain embodiments, the plurality of containers house a medicament. In certain embodiments, at least one of the containers houses a medicament in a nonstandard amount. In certain embodiments, at least two of the containers houses a medicament in a nonstandard amount.

In certain embodiments, the vessel is a bag. In certain embodiments, the vessel is a bottle. In certain embodiments, the containers are bottles or bags. In certain embodiments, the containers are vials.

In certain embodiments, the decontamination devices are integrally manufactured with the containers. In certain embodiments, the decontamination devices form a unitary structure with the containers.

In certain embodiments, the coupling between the plurality of containers and the vessel forms an airtight coupling.

In certain embodiments, a first container housing a drug or a pharmaceutically equivalent formulation thereof in an amount less than about 5% of a Table 1 Column A amount, in an amount less than about 10% of a Table 1 Column A amount, in an amount less than 20% of a Table 1 Column A amount, less than about 30% of a Table 1 Column A amount, or less than about 50% of a Table 1 Column A amount.

In certain embodiments, a second container houses a drug or a pharmaceutically equivalent formulation thereof in an amount less than about 5% of a Table 1 Column A amount, in an amount less than about 10% of a Table 1 Column A amount, in an amount less than 20% of a Table 1 Column A amount, less than about 30% of a Table 1 Column A amount, or less than about 50% of a Table 1 Column A amount.

In certain embodiments, a third container houses a drug or a pharmaceutically equivalent formulation thereof in an amount less than about 5% of a Table 1 Column A amount, in an amount less than about 10% of a Table 1 Column A amount, in an amount less than 20% of a Table 1 Column A amount, less than about 30% of a Table 1 Column A amount, or less than about 50% of a Table 1 Column A amount.

In certain embodiments, a fourth container houses a drug or a pharmaceutically equivalent formulation thereof in an amount less than about 5% of a Table 1 Column A amount, in an amount less than about 10% of a Table 1 Column A amount, in an amount less than 20% of a Table 1 Column A amount, less than about 30% of a Table 1 Column A amount, or less than about 50% of a Table 1 Column A amount.

In certain embodiments, a fifth container houses a drug or a pharmaceutically equivalent formulation thereof in an amount less than about 5% of a Table 1 Column A amount, in an amount less than about 10% of a Table 1 Column A amount, in an amount less than 20% of a Table 1 Column A amount, less than about 30% of a Table 1 Column A amount, or less than about 50% of a Table 1 Column A amount.

In certain embodiments, the system further comprises a second container housing a beneficial substance in a nonstandard or standard amount.

In other aspects, the present invention provides a vessel with plurality of entry ports flush mounted/surface mounted.

In other aspects, the present invention provides a system of vessels with plurality of entry ports+plurality of containers. In other aspects, the present invention provides a system of vessel with decontamination devices+plurality of containers. In other aspects, the present invention provides a system of vessel+plurality of containers with decontamination devices. In other aspects, the present invention provides a modular assembly system. In other aspects, the present invention provides a modular assembly system with decontamination devices. In other aspects, the present invention provides a syringe with plurality of ports. In other aspects, the present invention provides a syringe with plurality of decontamination devices.

Unless otherwise defined, all technical or/and scientific terms used herein have the same meaning as commonly understood by one of ordinary skill in the art to which the invention pertains. Although methods and materials similar or equivalent to those described herein can be used in the practice or testing of embodiments of the invention, exemplary methods or/and materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1E are schematic front cut view illustrations presenting an exemplary system which includes an IV bag having a plurality of entry ports and an exit port, wherein a plurality of containers are received and engaged by the plurality of entry ports to transfer an amount from the plurality of containers to the IV bag, according to some embodiments of the invention.

FIGS. 2A-2E are schematic front cut view illustrations presenting a further exemplary system which includes an IV bag having a plurality of entry ports and an exit port, wherein a plurality of containers are received and engaged by the plurality of entry ports to transfer a dose or amount of a beneficial substance from the plurality of containers to the IV bag, according to some embodiments of the invention.

FIGS. 3A-3E are schematic front cut view illustrations presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIGS. 4A-4D are schematic front cut view illustrations presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIGS. 5A-5D are schematic front cut view illustrations presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIGS. 6A-6D are schematic front cut view illustrations presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIGS. 7A-7D are schematic front cut view illustrations presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIGS. 8A-8D are schematic front cut view illustrations presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIG. 9 is a schematic front cut view illustration presenting a further exemplary system which includes an IV bag having a plurality of entry ports and an exit port, according to some embodiments of the invention.

FIG. 10 is a schematic front cut view illustration presenting a further exemplary system which includes an IV bag having a plurality of entry ports and an exit port, according to some embodiments of the invention.

FIG. 11 is a schematic front cut view illustration presenting a further exemplary system which includes an IV bag having a plurality of entry ports and an exit port, according to some embodiments of the invention.

FIGS. 12A-12E are schematic front cut view illustrations presenting a further exemplary system which includes an IV bag having a plurality of entry ports and an exit port, wherein a plurality of syringe elements are received and engaged by the plurality of entry ports to transfer a dose from the plurality of syringe elements to the IV bag, according to some embodiments of the invention.

FIGS. 13A-13H are schematic front cut view illustrations presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIG. 14 is schematic front cut view illustrations presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIG. 15 is a schematic front cut view illustration presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIG. 16 is a schematic front cut view illustration presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIGS. 17A-17D are schematic front cut view illustrations presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIGS. 18A-18D are schematic front cut view illustrations presenting a further exemplary system which includes a syringe having a plurality of entry ports, wherein a plurality of containers are received and engaged by the plurality of entry ports to transfer a dose from the plurality of containers to the syringe, according to some embodiments of the invention.

FIGS. 19A-19D are schematic front cut view illustrations presenting a further exemplary system which includes a syringe having a plurality of entry ports, wherein a plurality of containers are received and engaged by the plurality of entry ports to transfer a dose from the plurality of containers to the syringe, according to some embodiments of the invention.

FIGS. 20A-20D are schematic front cut view illustrations presenting a further exemplary system which includes a syringe having a plurality of entry ports and an exit port, wherein a plurality of containers are received and engaged by the plurality of entry ports to transfer a dose from the plurality of containers to the syringe, according to some embodiments of the invention.

FIGS. 21A-21E are schematic front cut view illustrations presenting a further exemplary system which includes a syringe having a plurality of entry ports, wherein a plurality of containers are received and engaged by the plurality of entry ports to transfer a dose from the plurality of containers to the syringe, according to some embodiments of the invention.

FIG. 22 is a schematic front cut view illustration presenting a further exemplary system which includes a syringe having a plurality of entry ports, showing various objects able to be received by the plurality of entry ports, according to some embodiments of the invention.

FIGS. 23A-23E are schematic front cut view illustrations presenting a further exemplary system which includes a syringe having a plurality of entry ports, wherein a plurality of containers are received and engaged by the plurality of entry ports to transfer a dose from the plurality of containers to the syringe, according to some embodiments of the invention.

FIGS. 24A-24E are schematic front cut view illustrations presenting a further exemplary system which includes a syringe having a plurality of entry ports, wherein a plurality of containers are received and engaged by the plurality of entry ports to transfer a dose from the plurality of containers to the syringe, according to some embodiments of the invention.

FIGS. 25A-25I are schematic front cut view illustrations presenting a further exemplary system which includes a syringe having a plurality of entry ports, wherein a plurality of containers are received and engaged by the plurality of entry ports to transfer a dose from the plurality of containers to the syringe, according to some embodiments of the invention.

FIGS. 26A-26K are schematic front cut view illustrations presenting a further exemplary system which includes a syringe having a plurality of entry ports, wherein a plurality of containers are received and engaged by the plurality of entry ports to transfer a dose from the plurality of containers to the syringe, according to some embodiments of the invention.

FIGS. 27A-27B are schematic front cut view illustrations presenting a further exemplary system which includes a modular dosing system of a plurality of containers, whereby the containers are configured to be connected to one another, according to some embodiments of the invention.

FIGS. 28A-28B are schematic front cut view illustrations presenting a further exemplary system which includes a modular dosing system of a plurality of containers, whereby the containers are configured to be connected to one another, according to some embodiments of the invention.

FIGS. 29A-29B are schematic front cut view illustrations presenting a further exemplary system which includes a modular dosing system of a plurality of containers, whereby the containers are configured to be connected to one another, according to some embodiments of the invention.

FIGS. 30A-30B are schematic front cut view illustrations presenting a further exemplary system which includes a modular dosing system of a plurality of containers, whereby the containers are configured to be connected to one another, according to some embodiments of the invention.

FIGS. 31A-31D are schematic front cut view illustrations presenting a further exemplary system which includes a modular dosing system of a plurality of containers, whereby the containers are configured to be connected to one another, according to some embodiments of the invention.

FIGS. 32A-32G are schematic front cut view illustrations presenting a further exemplary system which includes a modular dosing system of a plurality of containers, whereby the containers are configured to be connected to one another, according to some embodiments of the invention.

FIGS. 33A-33C are schematic front cut view illustrations presenting a further exemplary system which includes a modular dosing system of a plurality of containers, whereby the containers are configured to be connected to one another, according to some embodiments of the invention.

FIGS. 34A-34F are schematic front cut view illustrations presenting a further exemplary system which includes a modular dosing system of a plurality of containers, whereby the containers are configured to be connected to one another, according to some embodiments of the invention.

FIG. 35 is a schematic front cut view illustration presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIG. 36 is a schematic front cut view illustration presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIG. 37 is a schematic front cut view illustration presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIG. 38 is a schematic front cut view illustration presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIG. 39 is a schematic front cut view illustration presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIG. 40 is a schematic front cut view illustration presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIG. 41 is a schematic front cut view illustration presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIG. 42 is a schematic front cut view illustration presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIG. 43 is a schematic front cut view illustration presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention;

FIG. 44 is a schematic front cut view illustration presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIG. 45 is a schematic front cut view illustration presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIG. 46 is a schematic front cut view illustration presenting a further exemplary system which includes a modular dosing system of a plurality of containers, whereby the containers are configured to be connected to one another, according to some embodiments of the invention.

FIG. 47 is a schematic front cut view illustration presenting a further exemplary system which includes a modular dosing system of a plurality of containers, whereby the containers are configured to be connected to one another, according to some embodiments of the invention.

FIG. 48 is a schematic front cut view illustration presenting a further exemplary system which includes a modular dosing system of a plurality of containers, whereby the containers are configured to be connected to one another, according to some embodiments of the invention.

FIG. 49 is a schematic front cut view illustration presenting a further exemplary system which includes a modular dosing system of a plurality of containers, whereby the containers are configured to be connected to one another, according to some embodiments of the invention.

FIG. 50 is a schematic front cut view illustration presenting a further exemplary system which includes an IV bag having a plurality of entry ports and an exit port, showing various objects able to be received by the plurality of entry ports, according to some embodiments of the invention.

FIG. 51 is a schematic front cut view illustration presenting a further exemplary system which includes an IV bag having a plurality of entry ports and an exit port, showing various objects able to be received by the plurality of entry ports, according to some embodiments of the invention.

FIG. 52 is a schematic front cut view illustration presenting a further exemplary system which includes an IV bag having a plurality of entry ports and an exit port, showing various objects able to be received by the plurality of entry ports, according to some embodiments of the invention.

FIGS. 53A-53B is a schematic front cut view illustration presenting a further exemplary system which includes a modular dosing system of a plurality of containers showing amounts of dosing regimens, whereby the containers are configured to be connected to one another, according to some embodiments of the invention.

FIG. 54 is a schematic front cut view illustration presenting a further exemplary system which includes an IV bag having a plurality of entry ports and an exit port, wherein a plurality of containers are received and engaged by the plurality of entry ports to transfer a dose from the plurality of containers to the IV bag, according to some embodiments of the invention.

FIG. 55 is a schematic front cut view illustration presenting a further exemplary system which includes a syringe or container with an expulsion member having a plurality of entry ports and an exit port, wherein a plurality of containers are received and engaged by the plurality of entry ports to transfer a dose from the plurality of containers to the syringe or container with an expulsion member, according to some embodiments of the invention;

FIG. 56 is a schematic front cut view illustration presenting a further exemplary system which includes a bottle having a plurality of entry ports and an exit port, wherein a plurality of containers are received and engaged by the plurality of entry ports to transfer a dose from the plurality of containers to the bottle, according to some embodiments of the invention;

It should be appreciated that for simplicity and clarity of illustration, elements shown in the figures have not necessarily been drawn to scale. For example, the dimensions of some of the elements are exaggerated relative to each other for clarity. Further, where considered appropriate, reference numerals have been repeated among the figures to indicate corresponding elements.

### DETAILED DESCRIPTION OF THE INVENTION

It is understood that the invention is not limited to the particular methodology, devices, items or products etc., described herein, as these may vary as the skilled artisan will recognize. It is also to be understood that the terminology used herein is used for the purpose of describing particular embodiments only, and is not intended to limit the scope of the invention. The following exemplary embodiments may be described in the context of exemplary bedding articles for ease of description and understanding. However, the invention is not limited to the specifically described products and methods and may be adapted to various applications without departing from the overall scope of the invention. All ranges disclosed herein include the endpoints. The use of the term “or” shall be construed to mean “and/or” unless the specific context indicates otherwise.

The present invention relates, in some embodiments thereof, to systems, containers and substance transfer methods to establish fluid communication between medical vessels.

The present invention relates, in some embodiments thereof, to devices, methods and systems allowing an engagement of a vessel, such as an IV bag, syringe or bottle, with two or more containers in a sterile or decontaminated manner. In some embodiments of the invention, the systems and devices of the invention include a vessel comprising at least two entry ports and at least one exit port, and at least one container containing a dosing regimen, the at least one container configured to be received by one of the at least two entry ports of the vessel, whereby upon connection of the at least one container to the one of the at least two entry ports of the vessel, the dosing regimen in the at least one container is transferred into the vessel, thereby allowing the user to assemble virtually any customized dosage amount of a beneficial substance for an individual with maximum flexibility and without the need for manually manipulating and measuring the beneficial substance.

The present invention relates, in some embodiments thereof, to devices, methods and systems allowing an engagement of containers such as vials with one another in a modular configuration. In some embodiments of the invention, the systems and devices of the invention involve a modular dosing system for adding at least one dose of a medicament to a preparation in a modular construction.

The present invention relates to providing a “customized/individualized” final dose of an injectable or intracorporeally administered medication and how it provides flexibility to “assemble” a customized amount of a medicament in a pharmacy, doctor's office, and/or at the point of care.

The present invention relates, in some embodiments thereof, to devices, methods and systems allowing for displaying a dosing regimen or single dose of a medicament, so that the administrator of the dose is able to precisely ascertain the product identity and dose administered to a patient.

The present devices, methods and systems are particularly useful for medical purposes, wherein sterile preparations of medical substances are vastly needed, especially for customized dosage regimens requiring a plurality of packaged containers housing beneficial substances. In one embodiment, the present invention provides a vessel with multiple connection interfaces and entry ports, that may be coupled to, or integrally formed with, medical vessels or containers, such as vials, syringes, containers, bottles, etc.

The invention provides a solution to an unmet and long felt need in the medical setting and allows connecting in a sterile manner, a vessel with containers that house amounts of beneficial substances. The herein disclosed devices and systems are user friendly, cost effective and abolish the need for complicated and expensive known methods for transferring medical substances. In an embodiment of the invention, the herein disclosed devices and systems are disposable. In an embodiment of the invention, the herein disclosed devices and systems are non-disposable.

In an aspect of the invention, the devices and systems of the invention include a vessel, the vessel may be a bag, bottle, syringe, infusion line, connector, connector with a plurality of ports, a vial, a filter, manifold and/or any type of container and/or medicinal device used for the housing and/or administration of a medicinal substance to a patient. Additionally, the vessel is designed to be coupled with at least two containers containing a dose and subsequent delivery of the assembled substance to a patient for administration. Optionally, the vessel may be designed to couple with at least two connectors, and/or a plurality of connectors, wherein the connectors are configured to couple to at least two or a plurality of containers. Administration may be intracorporal administration to a patient. The medicinal substance may be a medicament, a nutritional substance, vitamins, minerals, elements, trace elements a fluid, a sterile fluid, a solid, a semi-solid, a lyophilized substance, a diluent, a diagnostic substance, a pharmaceutical substance, and/or etc.

In certain aspects, the vessel has at least two entry ports and at least one exit port collectively comprising a plurality of fluid communications. The entry ports and exit ports may be integrally/fixedly attached to the container. The entry ports and exit ports may form a unitary structure with the container. The entry ports may be flush mounted or surface mounted to the vessel. The entry ports may be adhered to the vessel via an adhesive or may be constructed to be within or integrally built into the vessel. The entry ports may abut the container. The entry ports may abut a wall of the container.

In some embodiments of the invention the ports are designed universally in that they can connect with all other universally designed types of ports on containers, vessels, and/or connectors.

Embodiments of the invention may include a modular assembly of a plurality of containers to a manifold, wherein the containers house non-standard and/or standard amounts of beneficial substances and/or medicaments allowing for assembly of a final customized amount of a beneficial substance and/or medicament. Optionally, the manifold may be connected to an infusion line, container or a syringe.

Embodiments of invention as shown in the below reference chart and include a modular assembly of containers to an IV bag in a first aspect; modular assembly of containers to a syringe in a second aspect; and modular assembly of containers to one another in a third aspect as shown in the reference chart below:

The application incorporates by reference various types of connection mechanisms and decontamination devices set forth in the following applications:
DEVICES AND SYSTEMS WITH AN EXTERNAL DISPLACEMENT MECHANISM FOR CONTAMINANT-FREE ENGAGEMENT OF PHARMACEUTICAL VESSELS AND PHARMACEUTICAL ADMINISTRATION DEVICES having U.S. application Ser. No. 16/100,594 filed Aug. 10, 2018;
DEVICES AND SYSTEMS WITH AN INTERNAL DISPLACMENT MECHANISM FOR CONTAMINANT-FREE ENGAGEMENT OF MEDICAL VESSELS AND DEVICES having U.S. application Ser. No. 16/100,712 filed Aug. 10, 2018;
SYSTEMS, DEVICES AND METHODS FOR DECONTAMINATING SURFACES OF PHARMACEUTICAL VESSELS AND PHARMACEUTICAL ADMINSTRATION DEVICES having U.S. application Ser. No. 16/100,840 filed Aug. 10, 2018;
DECONTAMINATION DEVICE FOR PHARMACEUTICAL VESSELS having U.S. application Ser. No. 16/100,964 filed Aug. 10, 2018.

The entry and exit ports may incorporate decontamination devices to provide for a sterile contaminant-free engagement between the plurality of fluid communications. Contaminant-free may also mean air particle-free engagement between vessels.

In certain embodiments, the vessel includes a plurality (meaning at least 2, at least 3, at least 4, at least 5, 6 or more) of entry and exit ports and provides for the assembly of a customized amount of a beneficial substance without requiring the user to manually manipulate (draw up, measure, compound, intermix, mix) a beneficial sub stance.

The system comprises providing a plurality of containers, the vessels may be vials, syringes or bottles housing a beneficial substance, such as a dosing regimen. The beneficial substance in each of the vials may be the same beneficial substance, may be the same beneficial substance in different amounts or in the same amount, or may be different beneficial substances.

For example, if a total parenteral nutrition is required to be prepared the user (usually a nurse/pharmacist/doctor) may simply attach a vial of multivitamins to a bag/bottle, then attach a vial containing a protein substance, then attach a vial containing elements/trace elements, then attach a vial containing insulin, then attach a vial containing any other needed substance to the bag/bottle. Instead of manually drawing up each of these beneficial substances in a syringe and visually measuring the amounts drawn up the user may simply attach a plurality of the mentioned vials to the vessel (bag/bottle/container) and then dispense the final product to a nurse or to the patient. No manual manipulation and measuring of the beneficial substances is required in a sterile environment.

The plurality of entry and exit ports may be located on a side of a vessel, on the top of the vessel, on the bottom of a vessel, and combination thereof. The vessel may be empty. The vessel may house a fluid. The fluid may be a sterile fluid. The fluid may be a diluent. The fluid may be a solution. The fluid may be a suspension. The vessel may have a negative pressure relative ambient air pressure, ambient air pressure at sea level, and/or atmospheric pressure. Optionally, the vessel may have a negative pressure relative a container and/or a plurality of containers that are intended to couple with the vessel.

In one or more embodiments, the herein disclosed invention allows transferring medical substances in a contaminant-free, or in a substantially contaminant-free manner.

In one or more embodiments, the herein disclosed invention affords an engagement of vessels with containers, containers with syringes, and containers with other containers in a contaminant-free, or in a substantially contaminant-free manner.

In one or more embodiments, the herein disclosed invention provides a fluidic passageway or communication between vessels with containers, containers with syringes, and containers with other containers in a contaminant-free, or in a substantially contaminant-free manner.

In one or more embodiments, the herein disclosed invention affords to isolate and/or entrap ambient air particles present between medical vessels and containers, containers with syringes, and containers with other containers, and between interfaces and other interfaces. In one or more embodiments, the herein disclosed invention, allows to substantially decrease the chances to introduce contaminants within a medical substance, when preparing medical substances for administration to patients.

As used herein the term “substances” refers to various types of materials that should be kept sterile. The substances may be liquid, semi-solid, solid, lyophilized solid or gas. In one or more embodiments, the substances are “medical substances”. As used herein the term “medical substances” refers to and encompasses any of the various medical drugs, fluids, nutritional products, liquids, solids, suspensions and the like.

As used herein the term “contaminant-free” is interchangeable with the term “sterile”, “disinfected”, and “decontaminated”. The term refers to substances that are free or substantially free of ambient air particles and/or pathogens. Typically, when less or no air is introduced within medical substances, the chances of contamination by pathogens, such as, bacteria, viruses, funguses, spores, pyrogens or the alike is completely abolished or significantly reduced.

As used herein, the term “substantially contaminant-free” means significantly less ambient air present when transferring medical substances with the herein disclosed vessels and systems, as compared to comparable conditions for transferring medical substances without the herein disclosed vessels and systems.

As used herein the term “ambient air particles” is interchangeable with the term “environmental air particles” and refers to air particles present in a non-filtered environment. For example, air can be purified by filters, such as a High Efficiency Particulate Air (HEPA) filter.

As used herein the term “connection interface” encompasses any surface, layer, plane or the alike that can be attached to a vessel. The term may encompass a structure that can be coupled or adhered to a vessel and that can engage with a complementary connection interface.

As used herein the term “external displacement” refers to a displacement (i.e., dislocation) of the herein disclosed first and/or second, and/or any additional connection interfaces. In an embodiment of the invention, the displacement is external, namely, outside the vessels being connected by the herein disclosed system. In an embodiment of the invention, the displacement is external to the fluid communication established following engagement of the vessels being connected by the herein disclosed system, methods and devices. In an embodiment of the invention, the displacement maintains hermetic seal of the connection interfaces and/or the vessels. The displacement may occur via a sliding motion, or by a pulling out motion, or by peeling the connections. In an embodiment of the invention, the displacement occurs for both the first and second connection interfaces. The external displacement may optionally occur simultaneously for both connection interfaces or may occur consecutively. In an embodiment of the invention, the external displacement established a fluid passageway between two or more vessels. In one or more embodiments, the connection interface is configured to hermetically seal an aperture present in a vessel or on a wall of a vessel. In one or more embodiments, at about the time or following the external displacement, the aperture of a vessel reseals, allowing a hermetic airtight connection between two or more vessels.

As used herein the term “internal displacement” refers to a displacement (i.e., dislocation) of the herein disclosed first and/or second connection interfaces. In an embodiment of the invention, the displacement is internal, namely, dislocation into one of the vessels associated with a connection interface. In an embodiment of the invention, the displacement is within the fluid passageway established following engagement of the herein disclosed vessels. In an embodiment of the invention, the internal displacement occurs for one or both the first and second connection interfaces. The internal displacement may optionally occur simultaneously for both connection interfaces or may occur consecutively. The displacement may occur via a pressure exerted on the exterior of the connection interfaces, a pressure exerted through a flexible wall of a vessel or container, a twisting of the connection interfaces with respect to one another, or an engagement of the interfaces with one another and actuation by a user.

As used herein the term “vessel” refers to any device utilized for containing substances as herein disclosed. In one or more embodiments, the vessel may be used for containing medical substances. In one or more embodiments, the vessel may be used for housing medical substances. In an embodiment of the invention, the vessel is a medical vessel. In an embodiment of the invention, the vessel is a medical device. In an embodiment of the invention, the vessels are used for, and adapted to allow connection to another vessel(s). In an embodiment of the invention, the vessel may be a medical container utilized for accommodating medical substances. Various types of medical containers are contemplated. The medical container may be selected, without limitation, from a vial, a bag, a chamber, a bottle, and the alike. In an embodiment of the invention, the term vessel further encompasses elements that can be used to connect between vessels. In accordance with this embodiment, the vessel may be selected, without limitation, from a connector, a connector with a plurality of openings, a connector with a plurality of ports, a port, a syringe, an infusion line, a tubing, a syringe, a filter, a spike, a port and a manifold. In an embodiment of the invention, one or more connection interfaces (for example, two or more, three or more, etc.) may be coupled to a first vessel and each of those connection interfaces may be coupled and engage with a second connection interface present on a second vessel. In one or more embodiments, the vessels to be engaged may have similar surface area or similar contact surface area (i.e., surface onto which the connection interface is coupled to). For example, a first vessel and a second vessel may have similar surface area or similar contact surface area. In one or more embodiments, the vessels to be engaged may have different surface area or different contact surface area. For example, a first connection interface may have a greater surface area or contact surface area than a second vessel. In an embodiment of the invention, one or more decontamination devices (for example, two or more, three or more, etc.) may be coupled to a first vessel and each of those decontamination devices may be coupled and engage with a container or a connector.

As used herein the term “fluid communication” refers to two or more vessels in which substances may pass therethrough either directly or indirectly. The fluid communication may occur via a fluid passageway that allows for the flow/transfer of substances.

As used herein the term “directly receives” refers to providing a substance to another container without an intermediary.

As used herein, the term “non-standard amount” is an amount or dosing regimen that is not available in a commercially prepackaged amount.

As used herein, the term “sub-therapeutic amount” is a dosing amount that is less than a therapeutic amount given to a patient.

As used herein, the term “dosing regimen(s)” refers to an amount of a beneficial substance. The term “dosing regimen(s)” and “amount” may be used interchangeably. In one or more embodiments, the beneficial substance may be a medicament, a nutritional substance and the alike.

As shown below, various drugs, or therapeutically equivalent drugs and/or formulations, that can be used in system and methods of the invention include, but are not limited to: Abciximab, Acetaminophen, Acetazolamide, Ado-trastuzumab, Aldesleukin, Alefacept, Alemtuzumab, Alfentanil, Allopurinol, Alprostadil, Amifostine, Aminocaproic Acid, Ammonium Chloride, Amoxicillin, Amsacrine, Antithymocyte Globulin Rabbit, Argatroban, Aripiprazole, Arsenic Trioxide, Asparaginase Erwinia Chrysanthemi, Atezolizumab, Azathioprine Sodium, Azithromycin, Baclofen, Benztropine, Bezlotoxumab, Bivalirudin, Blinatumomab, Bortezomib, Brentuximab, Bretylium Tosylate, Brivaracetam, Brodalumab, Buprenorphine, Busulfan, Calcitriol, Calcium Chloride, Canakinumab, Cangrelor, Capromab, Carbamazepine, Carmustine, Ceftolozane Sulfate/Tazobactam Sodium, Ceftolizumab, Chloramphenicol, Chlorothiazide, Cidofovir, Cladribine, Clarithromycin, Clonazepam, Colistimethate, Conivaptan, Cyclizine Lactate, Cyclosporine, Daclizumab, Dactinomycin, Dalbavancin, Dantrolene, Daptomycin, Daunorubicin, Denosumab, Diclofenac Sodium, Dinutuximab, Diphenhydramine, Doxapram, Dupilumab, Eculizumab, Edetate Calcium Disodium, Efalizumab, Ephedrine, Ertapenem, Estrogens Conjugated, Ethacrynate Sodium, Floxuridine, Fludarabine, Fluphenazine Hydrochloride, Folic Acid, Fomepizole, Fosaprepitant Dimeglumine, Foscarnet, Fusidate Sodium, Gallium, Ganciclovir, Gemcitabine, Gemtuzumab, Gentamicin Pediatric Formulation, Golimumab, Guselkumab, Hyaluronidase, Hydralazine, Ibandronate Sodium, Ibuprofen Lysinate, Ibutilide, Ibritumomab Tiuxetan, Idarucizumab, Indomethacin Sodium Trihydrate, Infliximab, Iodipamide Meglumine 52%, Isavuconazium sulfate, Ixekizumab, Lepirudin, Mechlorethamine Hydrochloride, Melphalan Hydrochloride, Mepolizumab, Mesna, Methadone, Methocarbamol, Methohexital Sodium, Methotrimeprazine Hydrochloride, Methyldopate Hydrochloride, Metoprolol, Metronidazole, Mexilitene Hydrochloride, Mitoxantrone, Moxifloxacin, Multivitamins, Mycophenolate, Natalizumab, Necitumumab, Nesiritide, Nicardipine, Norepinephrine, Obiltoxaximab, Obinutuzumab, Ocrelizumab, Olaratumab, Omaliumab, Omeprazole, Oritavancin Diphosphate, Pantoprazole, Pemetrexed, Penicillin G Potassium, Pentamidine Isethionate, Pentazocine, Pentostatin, Peramivir, Pertuzumab, Phentolamine Mesylate, Polymyxin B Sulfate, Posaconazole, Pralidoxime, Procainamide, Propranolol Hydrochloride, Quinidine Gluconate, Raxibacumab, Reslizumab, Reteplase, Rifampin, Salbutamol, Sargramostim, Scopolamine Butylbromide, Secukinumab, Sodium Ferric Gluconate Complex, Sodium Lactate, Sodium Nitroprusside, Sodium Thiosulfate, Streptomycin Sulfate, Streptozocin, Tacrolimus, Tedizolid Phosphate, Teniposide, Terbutaline Sulfate, Tetracaine Hydrochloride, Thiotepa, Ticarcillin Disodium/Clavulanate Potassium, Tigecycline, Topotecan Hydrochoride, Tranexamice Acid, Ustekinumab Intravenous Formulation, Valproate, Vedolizumab, Vinblastine Sulfate, Vincristine, Vincristine Sulfate Liposomal, Vitamin A, Voriconazole, Zidovudine, Zoledronic Acid, Bacitracin, Chromium (chromic chloride injection), Copper (cupric chloride injection), Carfilzomib

As shown below is a table of injectable drugs and currently commercially available packaged amounts the drugs are supplied in by drug manufacturers. The amounts listed below in Table 1 are referred to as “standard amounts” as the drug manufacturers and the Food and Drug Administration have decided to commercially supply and give regulatory approval for the following amounts. The below drugs may be prepared and administered to patients using embodiments of the present invention:

TABLE 1

| DRUG | COLUMN A | | COLUMN B | | COLUMN C | | COLUMN D | |
|---|---|---|---|---|---|---|---|---|
| Acetycysteine | 3 | gm | 6 | gm | | | | |
| Acyclovir | 500 | mg | 1 | gm | | | | |
| Adalimumab | 10 | mg | 20 | mg | 40 | mg | 80 | mg |
| Adalimumab-atto | 20 | mg | 40 | mg | | | | |
| Adenosine | 60 | mg | 90 | mg | 3 | gm | | |
| Albumin 5% | | | | | | | | |
| Albumin 20% | | | | | | | | |
| Albumin 25% | 50 | ml | 100 | ml | | | | |
| Alirocumab | 75 | mg | 150 | mg | | | | |
| Alteplase | 2 | mg | 50 | mg | 100 | mg | | |
| Amikacin | 100 | mg | 500 | mg | 1 | gm | | |
| Aminophylline | 250 | mg | 500 | mg | | | | |
| Amiodaroine | 150 | mg | 450 | mg | 900 | mg | | |
| Amoxicillin/Clavulanate | 600 | mg | 1.2 | gm | | | | |
| Amphotericin B | 50 | mg | 100 | mg | | | | |
| Amphotericin B Cholesteryl | 50 | mg | 100 | mg | | | | |
| Sulfate Complex | | | | | | | | |
| Amphotericin B Liposomal | 50 | mg | 100 | mg | | | | |
| Ampicillin | 125 | mg | 250 | mg | 500 | mg | 1 | gm |
| Ampicillin Sodium/Sulbactam | 1.5 gm | | 3 gm | | | | | |
| Sodium | (1 gm/0.5 gm) | | (2 gm/1 gm) | | | | | |
| Anidulafungin | 50 | mg | 100 | mg | | | | |
| Anti-Hemophilic Factor | 250 | IU | 500 | IU | 1000 | IU | 3000 | IU |
| Ascorbic Acid | 500 | mg | 1 | gm | | | | |
| Atracurium | 50 | mg | 100 | mg | | | | |
| Atropine | 0.4 | mg | 0.5 | mg | 1 | mg | | |
| Aztreonam | 500 | mg | 1 | gm | 2 | gm | | |
| Basiliximab | 10 | mg | 20 | mg | | | | |
| Belimumab | 120 | mg | 400 | mg | | | | |
| Caffeine Citrate | 30 | mg | 60 | mg | | | | |
| Bendamustine | 25 | mg | 100 | mg | | | | |
| Bevacizumab | 100 | mg | 400 | mg | | | | |
| Bleomycin | 15 | units | 30 | units | | | | |
| Bumetanide | 0.5 | mg | 1 | mg | 2.5 | mg | | |
| Bupivacaine PERCENTAGE | 0.25 | mg | 0.5 | mg | 7.5 | mg | | |
| Butorphanol | 1 | mg | 2 | mg | 4 | mg | 20 | mg |
| Calcium Gluconate 10% | 10 | ml | 50 | ml | 100 | ml | | |
| Carboplatin | 50 | mg | 150 | mg | 450 | mg | 600 | mg |
| Caspofungin | 50 | mg | 70 | mg | | | | |
| Cefoperazone | 1 | gm | 2 | gm | 10 | gm | | |
| Cefazolin | 500 | mg | 1 | gm | 10 | gm | 20 | gm |
| Cefepime | 500 | mg | 1 | gm | 2 | gm | | |
| Cefotaxime | 500 | mg | 1 | gm | 2 | gm | 10 | gm |
| Cefotetan | 1 | gm | 2 | gm | 10 | gm | | |
| Cefoxitin | 1 | gm | 2 | gm | 10 | gm | | |
| Ceftaroline | 400 | mg | 600 | mg | | | | |
| Ceftazidime | 500 | mg | 1 | gm | 2 | gm | 6 | gm |
| Ceftriaxone | 250 | mg | 500 | mg | 1 | gm | 2 | gm |
| Cefuroxime | 750 | mg | 1.5 | gm | 7.5 | gm | | |
| Cetuximab | 100 | mg | 200 | mg | | | | |
| Chlorpromazine | 25 | mg | 50 | mg | | | | |
| Cimetadine | 300 | mg | ???? | | | | | |
| Ciprofloxacin | 200 | mg | 400 | mg | | | | |
| Cisatracurium | 10 | mg | 20 | mg | 200 | mg | | |
| Cisplatin | 50 | mg | 100 | mg | 200 | mg | | |
| Cleviprex | 25 | mg | 50 | mg | | | | |
| Clindamycin | 300 | mg | 600 | mg | 900 | mg | | |
| Clonidine | 1 | mg | 5 | mg | | | | |
| Cloxacillin Sodium | 250 | mg | 500 | mg | 1 | gm | 2 | gm |
| Cyanocobalamin | 100 | mcg | 1 | mg | | | | |
| Cyclophosphamide | 500 | mg | 1 | gm | 2 | gm | | |
| Cytarabine | 100 | mg | 500 | mg | 1 | gm | | |
| Dacarbazine | 100 | mg | 200 | mg | | | | |
| Daratumumab | 100 | mg | 400 | mg | | | | |
| Deferoxamine | 500 | mg | 2 | gm | | | | |
| Defibrotide | | | | | | | | |
| Desmopressin Acetate | 4 | mcg | 40 | mcg | | | | |
| Dexamethasone sodium | 4 | mg | 10 | mg | | | | |
| Phosphate | | | | | | | | |
| Dexmedetomidine | 200 | mcg | 400 | mcg | | | | |
| Hydrochloride | | | | | | | | |
| Dexrazoxane | 250 | mg | 500 | mg | | | | |
| Dextrose 50% | 50 | ml | 500 | ml | 1 | Liter | | |
| Diamorphine | 10 | mg | 30 | mg | 100 | mg | 500 | mg |
| Diazepam | 10 | mg | 50 | mg | | | | |
| Digoxin | 0.25 | mg | 0.5 | mg | | | | |
| Diltiazem | 25 | mg | 50 | mg | 125 | mg | | |
| Dimenhydrinate | 50 | mg | 500 | mg | | | | |
| Dobutamine | 250 | mg | 500 | mg | 1250 | mg | | |
| Docetaxel | 20 | mg | 40 | mg | 80 | mg | | |
| Dolasetron | 12.5 | mg | 100 | mg | | | | |
| Dopamine | 200 | mg | 400 | mg | 800 | mg | | |
| Doripenem | 250 | mg | 500 | mg | | | | |
| Doxorubicin | 20 | mg | 50 | mg | | | | |
| Doxycycline Hyclate | 100 | mg | 200 | mg | | | | |
| Droperidol | 2.5 | mg | 5 | mg | | | | |
| Durvalumab | 120 | mg | 500 | mg | | | | |
| Elotuzumab | 300 | mg | 400 | mg | | | | |
| Enalaprilat | 1.25 | mg | 2.5 | mg | | | | |
| Epirubicin | 50 | mg | 200 | mg | | | | |
| Epoprostenol Sodium | 0.5 | mg | 1.5 | mg | | | | |
| Eptifibatide | 20 | mg | 75 | mg | 200 | mg | | |
| Erythromycin Lactobionate | 500 | mg | 1 | gm | | | | |
| Esmolol | 100 | mg | 2 | gm | 2.5 | gm | | |
| Esomeprazole | 20 | mg | 40 | mg | | | | |
| Etomidate | 20 | mg | 40 | gm | | | | |
| Etoposide | 100 | mg | 500 | mg | 1 | gm | | |
| Evolocumab | 140 | mg | 420 | mg | | | | |
| Famotidine | 20 | mg | 40 | mg | 200 | mg | | |
| Fenoldopam | 10 | mg | 20 | mg | | | | |
| Fentanyl | 100 | mcg | 250 | mcg | 500 | mcg | 1000 | mcg |
| Filgrastim | 300 | mcg | 480 | mcg | | | | |
| Floxacillin | 250 | mg | 500 | mg | 1 | gm | | |
| Fluconazole | 200 | mg | 400 | mg | | | | |
| Flumazenil | 0.5 | mg | 1 | mg | | | | |
| Fluorouracil (5-Fluorouracil) | 500 | mg | 1 | gm | 2.5 | gm | 5 | gm |
| Fosphenytoin | 100 | mgPE | 500 | mgPE | | | | |
| Furosemide | 20 | mg | 40 | mg | 100 | mg | | |
| Gentamicin (Adult Formulation) | 80 | mg | 800 | mg | | | | |
| Glycopyrrolate | 0.2 | mg | 0.4 | mg | 1 | mg | 4 | mg |
| Granisetron | 1 | mg | 4 | mg | | | | |
| Haloperidol Lactate | 5 | mg | 50 | mg | | | | |
| Hydrocortisone Sodium | 100 | mg | 250 | mg | 500 | mg | 1 | gm |
| Succinate | | | | | | | | |
| Hydromorphone Hydrochloride | | | | | | | | |
| Hydroxyzine | 25 | mg | 50 | mg | 100 | mg | 250 | mg |
| Ibuprofen - Caldolor brand | 400 | mg | 800 | mg | | | | |
| Idarubicin | 5 | mg | 10 | mg | 20 | mg | | |
| Ifosfamide | 1 | gm | 3 | gm | | | | |
| Imipenam/Cilastatin Sodium | 250 | mg | 500 | mg | | | | |
| Immune Globulin Human | ???? | | | | | | | |
| Iron Dextran | 50 | mg | 100 | mg | | | | |
| Insulin Regular | 30 | units | 100 | units | | | | |
| Interferone Alfa-2b | 10 million IU | | 18 million IU | | 50 million IU | | | |
| Iodixanol | 270 | mg | 550 | mg | 320 | mg | | |
| Iohexol | | | | | | | | |
| Iopamidol | | | | | | | | |
| Iothalmate Meglumine | | | | | | | | |
| Ioxaglate Meglumine and | | | | | | | | |
| Ioxaglate Sodium | | | | | | | | |
| Ipilimumab | 50 | mg | 200 | mg | | | | |
| Ironotecan | 40 | mg | 100 | mg | | | | |
| Isoproterenol | 0.2 | mg | 1 | mg | | | | |
| Isosorbide Dinitrate | 10 | mg | 50 | mg | 100 | mg | | |
| OTHER IRON PRODUCTS | | | | | | | | |
| Ketamine | 50 | mg | 100 | mg | 200 | mg | 250 | mg |
| Ketorolac | 15 | mg | 30 | mg | 60 | mg | | |
| Labetalol | 20 | mg | 40 | mg | 100 | mg | 200 | mg |
| Lenograstim | 13.4 million IU | | 33.6 million IU | | | | | |
| Leucovorin | 50 | mg | 100 | mg | 200 | mg | 350 | mg |
| Levetiracetam | 500 | mg | 1 | gm | 1.5 | gm | | |
| Levofloxacin | 250 | mg | 500 | mg | 750 | mg | | |
| Levothyroxine | 100 | mcg | 200 | mcg | 500 | mcg | | |
| Lidocaine Hydrochloride | 50 | mg | 100 | mg | 500 | mg | 1 | gm |
| Lincomycin | 600 | mg | 3 | gm | | | | |
| Linezolild | 200 | mg | 400 | mg | 600 | mg | | |
| Lorazepam | 2 | mg | 4 | mg | 20 | mg | 40 | mg |
| Magnesium Sulfate | | | | | | | | |
| Mannitol | | | | | | | | |
| Meropenem | 500 | mg | 1 | gm | | | | |
| Methotrexate | 50 | mg | 1 | gm | | | | |
| Methylprednisolone Acetate | 40 | mg | 80 | mg | 100 | mg | 200 | mg |
| Methylprednisolone Sodium | 40 | mg | 125 | mg | 500 | mg | 1 | gm |
| Succinate | | | | | | | | |
| Metoclopramide Hydrochloride | 10 | mg | 50 | mg | 150 | mg | | |
| Micafungin | 50 | mg | 100 | mg | | | | |
| Midazolam | 5 | mg | 10 | mg | 25 | mg | 50 | mg |
| Milrinone | 10 | mg | 20 | mg | 40 | mg | 50 | mg |
| Mitomycin | 5 | mg | 20 | mg | 40 | mg | 50 | mg |
| Nafcillin | 1 | gm | 2 | gm | | | | |
| Nalbuphine Hydrochloride | 10 | mg | 20 | mg | 100 | mg | 200 | mg |
| Naloxone | 0.04 | mg | 0.4 | mg | 2 | mg | 4 | mg |
| Neostigmine | 5 | mg | 10 | mg | | | | |
| Nimodipine | 10 | mg | 50 | mg | | | | |
| Nitroglycerin | 25 | mg | 50 | mg | | | | |
| Nivolumab | 40 | mg | 100 | mg | | | | |
| Ofatumumab | 100 | mg | 1000 | mg | | | | |
| Octreotide | 50 | mcg | 100 | mcg | 200 | mcg | 500 | mcg |
| Ondansetron | 4 | mg | 40 | mg | | | | |
| Oxacillin | 1 | gm | 2 | gm | | | | |
| Oxaliplatin | 50 | mg | 100 | mg | 200 | mg | | |
| Oxycodone Hydrochloride | 10 | mg | 20 | mg | | | | |
| Oxytocin | 10 | units | 100 | units | 300 | units | 500 | units |
| Paclitaxel | 30 | mg | 150 | mg | 300 | mg | | |
| Palivizumab | 50 | mg | 100 | mg | | | | |
| Palonsetron Hydrochloride | 0.075 | mg | 0.25 | mg | | | | |
| Pancuronium Bromide | 4 | mg | 10 | mg | | | | |
| Panitumumab | 100 | mg | 200 | mg | 400 | mg | | |
| Papaverine | 60 | mg | 300 | mg | | | | |
| Pembrolilzumab | 50 | mg | 100 | mg | | | | |
| Pentobarbital Sodium | 1 | gm | 2.5 | gm | | | | |
| Phenobarbital | 30 | mg | 60 | mg | 65 | mg | 130 | mg |
| Phenylephrine Hydrochloride | 10 | mg | 50 | mg | 100 | mg | | |
| Phenytoin Sodium | 100 | mg | 250 | mg | | | | |
| Phytonadione | 1 | mg | 10 | mg | 50 | mg | | |
| Piperacillin | 2.25 gm | | 3.375 gm | | 4.5 gm | | | |
| Sodium/Tazobactam Sodium | (2 gm/250 mg) | | (3 gm/750 mg) | | (4 gm/500 mg) | | | |
| Potassium Acetate | 40 | mEq | 100 | mEq | 200 | mEq | | |
| Potassium Chloride | 15 | mEq | 20 | mEq | | | | |
| Potassium Phosphates | 15 | mMol | 45 | mMol | | | | |
| Prochlorperazine Edisylate | 10 | mg | 50 | mg | | | | |
| Promethazine Hydrochloride | 25 | mg | 50 | mg | | | | |
| Propofol | 200 | mg | 500 | mg | 1 | gm | | |
| Protamine Sulfate | 50 | mg | 250 | mg | | | | |
| Pyridoxine Hydrochloride | 100 | mg | 1 | gm | 3 | gm | | |
| Quinpristin/Dalfopristin | 500 | mg | 600 | mg | | | | |
| Ramucirumab | 100 | mg | 500 | mg | | | | |
| Ranibizumab | 6 | mg | 10 | mg | | | | |
| Ranitidine Hydrochloride | 50 | mg | 150 | mg | 1 | gm | | |
| Rasburicase | 1.5 | mg | 7.5 | mg | | | | |
| Remifentanil Hydrochloride | 1 | mg | 2 | mg | 5 | mg | | |
| Rituximab | 100 | mg | 500 | mg | | | | |
| Rituximab Hyaluronidase | 1400 mg/23,400 | | 1600 mg/26,800 | | | | | |
| | units | | units | | | | | |
| Rocuronium Bromide | 50 | mg | 100 | mg | | | | |
| Ropivacaine Hydrochloride | 2 | mg | 5 | mg | 7.5 | mg | 200 | mg |
| Scopolamine Hydrobromide | 0.4 | mg | 1 | mg | | | | |
| Iron Sucrose | 100 | mg | 200 | mg | | | | |
| Siltuximab | 100 | mg | 400 | mg | | | | |
| Sodium Acetate | 40 | mEq | 100 | mEq | 200 | mEq | 400 | mEq |
| Sodium Bicarbonate | VARIOUS | | | | | | | |
| | SIZES | | | | | | | |
| Sodium Chloride | VARIOUS | | | | | | | |
| | SIZES | | | | | | | |
| Sodium Phosphates | 15 | mMol | 45 | mMol | 150 | mMol | | |
| Somatropin | 1.5 mg-24 mg | | | | | | | |
| Sorilumab | 150 | mg | 200 | mg | | | | |
| Succinylcholine Chloride | 100 | mg | 200 | mg | | | | |
| Sufentanil Citrate | 50 | mcg | 100 | mcg | 250 | mcg | | |
| Sugammadex Sodium | 200 | mg | 500 | mg | | | | |
| Sumatriptan Succinate | 4 | mg | 6 | mg | | | | |
| Teicoplanin | 200 | mg | 400 | mg | | | | |
| Telavancin Hydrochloride | 250 | mg | 750 | mg | | | | |
| Tenoxicam | 20 | mg | 40 | mg | | | | |
| Theophylline | VARIOUS | | | | | | | |
| | SIZES | | | | | | | |
| Thiamine Hydrochloride | 100 | mg | 200 | mg | | | | |
| Tirofigan Hydrochloride | 5 | mg | 12.5 | mg | | | | |
| Tobramycin Sulfate Pediatric | 20 | mg | | | | | | |
| Formulation | | | | | | | | |
| Tobramycin Sulfate Adult | 60 | mg | 80 | mg | 2 | gm | | |
| Formulation | | | | | | | | |
| Tocilizumab | 80 | mg | 200 | mg | 400 | mg | | |
| Torsemide | 20 | mg | 50 | mg | | | | |
| Tramadol Hydrochloride | 50 | mg | 100 | mg | | | | |
| Trastuzumab | 150 | mg | 420 | mg | | | | |
| Treprostinil Sodium | 10 | mg | 25 | mg | 50 | mg | 200 | mg |
| Trimethobenzamide | 200 | mg | 2 | gm | | | | |
| Trimethoprim/Sulfamethoxazole | 80 mg/400 mg | | 160 mg/800 mg | | 480 mg/2400 mg | | | |
| Tropisetron Hydrochloride | 2 | mg | 5 | mg | | | | |
| Vancomycin Hydrochloride | 500 | mg | 1 | gm | 5 | gm | 10 | gm |
| Vasopressin | 20 | units | | | | | | |
| Vecuronium | 10 | mg | 20 | mg | | | | |
| Verapamil Hydrochloride | 5 | mg | 10 | mg | | | | |
| Vincristine Sulfate | 1 | mg | 2 | mg | | | | |
| Vinorelbine Tartrate | 10 | mg | 50 | mg | | | | |
| Ziconotide Acetate | 100 | mcg | 200 | mcg | 500 | mcg | | |
| Ziv-Aflibercept | 100 | mg | 200 | mg | | | | |

As shown below is a table of injectable drugs and currently commercially available packaged amounts the drugs are supplied in by drug manufacturers. The amounts listed below in Table 2 are referred to as “standard amounts” as the drug manufacturers and the Food and Drug Administration have decided to commercially supply and give regulatory approval for the following amounts. The below drugs may be prepared and administered to patients using embodiments of the present invention:

TABLE 2

| | |
|---|---|
| | Commercially |
| Injectable | Available Amounts |
| Medicament | Column A Amount |

| | | |
|---|---|---|
| Abciximab | 2 | mg |
| Acetaminophen | 1000 | mg |
| Acetazolamide | 500 | mg |
| Ado-trastuzumab | 160 | mg |

| | |
|---|---|
| Aldesleukin | 22 million IU |

| | | |
|---|---|---|
| Alefacept | 15 | mg |
| Alemtuzumab | 30 | mg |
| Alfentanil | 2 | ml |
| Allopurinol | 500 | mg |
| Alprostadil | 500 | mcg |
| Amifostine | 500 | mg |
| Aminocaproic Acid | 5 | gm |
| Ammonium Chloride | 100 | mEq |
| Amoxicillin | 250 | mg |
| Amsacrine | 75 | mg |
| Antithymocyte | 25 | mg |
| Globulin Rabbit | | |
| Argatroban | 250 | mg |
| Aripiprazole | 9.75 | mg |
| Arsenic Trioxide | 10 | mg |
| Asparaginase Erwinia | 10,000 | IU |
| Chrysanthemi | | |
| Atezolizumab | 1200 | mg |
| Azathioprine Sodium | 100 | mg |
| Azithromycin | 500 | mg |
| Baclofen | 10 | mg |
| Benztropine | 2 | mg |
| Bezlotoxumab | 1000 | mg |
| Bivalirudin | 250 | mg |
| Blinatumomab | 35 | mcg |
| Bortezomib | 3.5 | mg |
| Brentuximab | 50 | mg |
| Bretylium Tosylate | 500 | mg |
| Brivaracetam | 500 | mg |
| Brodalumab | 210 | mg |
| Buprenorphine | 0.3 | mg |
| Busulfan | 6 | mg |
| Calcitriol | 1 | mcg |
| Calcium Chloride | 1 | gm |
| Canakinumab | 150 | mg |
| Cangrelor | 50 | mg |
| Capromab | 0.5 | mg |

Carbamazepine

| | | |
|---|---|---|
| Carmustine | 100 | mg |

| | |
|---|---|
| Ceftolozane | 1.5 gm (1 gm/0.5 gm) |
| Sulfate/Tazobactam | |
| Sodium | |

| | | | |
|---|---|---|---|
| Ceftolizumab | 200 | mg | |
| Chloramphenicol | 1 | gm | |
| Chlorothiazide | 500 | mg | |
| Cidofovir | 375 | mg | |
| Cladribine | 10 | mg | |
| Clarithromycin | 500 | mg | |
| Clonazepam | 1 | mg | |
| Colistimethate | 150 | mg | |
| Conivaptan | 20 | mg | |
| Cyclizine Lactate | 50 | mg | |
| Cyclosporine | 250 | mg | |
| Daclizumab | 25 | mg | 150 mg |
| Dactinomycin | 0.5 | mg | |
| Dalbavancin | 500 | mg | |
| Dantrolene | 500 | mg | |
| Daptomycin | 500 | mg | |
| Daunorubicin | 20 | mg | |
| Denosumab | 60 | mg | |
| Diclofenac Sodium | 37.5 | mg | |
| Dinutuximab | 17.5 | mg | |
| Diphenhydramine | 50 | mg | |
| Doxapram | 20 | mg | |
| Dupilumab | 300 | mg | |
| Eculizumab | 300 | mg | |
| Edetate Calcium | 500 | mg | |
| Disodium | | | |
| Efalizumab | 125 | mg | |
| Ephedrine | 50 | mg | |
| Ertapenem | 1 | gm | |
| Estrogens Conjugated | 25 | mg | |
| Ethacrynate Sodium | 50 | mg | |
| Floxuridine | 500 | mg | |
| Fludarabine | 50 | mg | |
| Fluphenazine | 25 | mg | |
| Hydrochloride | | | |
| Folic Acid | 50 | mg | |
| Fomepizole | 1.5 | mg | |
| Fosaprepitant | 150 | mg | |
| Dimeglumine | | | |
| Foscarnet | 6 | gm | |
| Fusidate Sodium | 500 | mg | |
| Gallium | 500 | mg | |
| Ganciclovir | 500 | mg | |
| Gemcitabine | 200 | mg | |
| Gemtuzumab | 5 | mg | |
| Gentamicin Pediatric | 20 | mg | |
| Formulation | | | |
| Golimumab | 50 | mg | |
| Guselkumab | 100 | mg | |
| Hyaluronidase | 150 | units | |
| Hydralazine | 20 | mg | |
| Ibandronate Sodium | 3 | mg | |
| Ibuprofen Lysinate | 20 | mg | |
| Ibutilide | 1 | mg | |
| Ibritumomab | 3.2 | mg | |
| Tiuxetan | | | |
| Idarucizumab | 2.5 | gm | |
| Indomethacin Sodium | 1 | mg | |
| Trihydrate | | | |
| Infliximab | 100 | mg | |
| Iodipamide | 20 | ml | |
| Meglumine 52% | | | |

| | |
|---|---|
| Isavuconazium sulfate | 300 mg sulfate = |
| | 200 mg |

| | | |
|---|---|---|
| Ixekizumab | 80 | mg |
| Lepirudin | 50 | mg |
| Mechlorethamine | 10 | mg |
| Hydrochloride | | |
| Melphalan | 10 | mg |
| Hydrochloride | | |
| Mepolizumab | 100 | mg |
| Mesna | 1 | gm |
| Methadone | 200 | mg |
| Methocarbamol | 1 | gm |
| Methohexital Sodium | 200 | mg |
| Methotrimeprazine | 25 | mg |
| Hydrochloride | | |
| Methyldopate | 250 | mg |
| Hydrochloride | | |
| Metoprolol | 5 | mg |
| Metronidazole | 500 | mg |
| Mexilitene | 250 | mg |
| Hydrochloride | | |
| Mitoxantrone | 2 | mg |
| Moxifloxacin | 400 | mg |
| Multivitamins | 5 | ml |
| Mycophenolate | 500 | mg |
| Natalizumab | 300 | mg |
| Necitumumab | 800 | mg |
| Nesiritide | 1.5 | mg |
| Nicardipine | 25 | mg |
| Norepinephrine | 4 | mg |
| Obiltoxaximab | 600 | mg |
| Obinutuzumab | 1 | gm |
| Ocrelizumab | 300 | mg |
| Olaratumab | 500 | mg |
| Omaliumab | 150 | mg |
| Omeprazole | 40 | mg |
| Oritavancin | 400 | mg |
| Diphosphate | | |
| Pantoprazole | 40 | mg |
| Pemetrexed | 500 | mg |

| | |
|---|---|
| Penicillin G | 5 million units |
| Potassium | |

| | | | |
|---|---|---|---|
| Pentamidine | 300 | mg | |
| Isethionate | | | |
| Pentazocine | 30 | mg | |
| Pentostatin | 10 | mg | |
| Peramivir | 10 | mg | |
| Pertuzumab | 420 | mg | |
| Phentolamine | 5 | mg | |
| Mesylate | | | |
| Polymyxin B Sulfate | 500,000 | units | |
| Posaconazole | 300 | mg | |
| Pralidoxime | 1 | gm | |
| Procainamide | 1 | gm | |
| Propranolol | 1 | mg | |
| Hydrochloride | | | |
| Quinidine Gluconate | 800 | mg | |
| Raxibacumab | 1700 | mg | |
| Reslizumab | 100 | mg | |
| Reteplase | 10.4 | units | 18.1 mg |
| Rifampin | 600 | mg | |
| Salbutamol | 500 | mcg | |
| Sargramostim | 250 | mcg | |
| Scopolamine | 20 | mg | |
| Butylbromide | | | |
| Secukinumab | 150 | mg | |
| Sodium Ferric | 62.5 | mg | |
| Gluconate Complex | | | |
| Sodium Lactate | 50 | mEq | |
| Sodium Nitroprusside | 50 | mg | |
| Sodium Thiosulfate | 12.5 | gm | |
| Streptomycin Sulfate | 1 | gm | |
| Streptozocin | 1 | gm | |
| Tacrolimus | 5 | mg | |
| Tedizolid Phosphate | 200 | mg | |
| Teniposide | 50 | mg | |
| Terbutaline Sulfate | 1 | mg | |
| Tetracaine | 20 | mg | |
| Hydrochloride | | | |
| Thiotepa | 15 | mg | |

| | |
|---|---|
| Ticarcillin | 3.1 gm |
| Disodium/Clavulanate | (3 mg/100 mg) |
| Potassium | |

| | | |
|---|---|---|
| Tigecycline | 50 | mg |
| Topotecan | 4 | mg |
| Hydrochoride | | |
| Tranexamice Acid | 1 | gm |
| Ustekinumab | 130 | mg |
| Intravenous | | |
| Formulation | | |
| Valproate | 500 | mg |
| Vedolizumab | 300 | mg |
| Vinblastine Sulfate | 10 | mg |
| Vincristine | | |
| Vincristine Sulfate | 5 | mg |
| Liposomal | | |
| Vitamin A | 100,000 | IU |
| Voriconazole | 200 | mg |
| Zidovudine | 200 | mg |
| Zoledronic Acid | 4 | mg |
| Bacitracin | 50,000 | units |
| Chromium (chromic | 40 | mcg |
| chloride injection) | | |
| Copper (cupric | 4 | mg |
| chloride injection) | | |
| Carfilzomib | 1000 | mg |

“mg” milligrams
“IU” international units
“ml” milliliters
“mcg” micrograms
“gm” grams
“mEq” milliequivalents

The list of drugs set forth above are non-exhaustive and other drugs can be administered via embodiments of the present invention. Moreover, the drugs can be provided in a commercially packaged unit and then administered to patients using embodiments of the present invention.

The present invention is counterintuitive with current FDA thinking and guidance which states that drug manufacturers should package drugs in amounts that would typically provide one dose to a patient. The present invention requires drug manufacturers to package medicaments and beneficial substance in amounts that would typically require the use of more than one or a plurality of vials/containers to provide a typical single dose to a patient. FDA recommends that a drug product's vial fill size should be appropriate for the labeled use of the product. FDA may request justification when there are questions about the proposed labeled fill sizes in an application. When deciding what is appropriate, applicants should consider the following as set forth in the U.S. Department of Health and Human Services Food and Drug Administration Center for Drug Evaluation and Research (CDER) Center for Biologics Evaluation and Research (CBER) June 2015 Pharmaceutical Quality/CMC:
Single dose vials should not contain a significant volume beyond what would be considered a usual or maximum dose for the expected use of the drug product.
Consumers and/or healthcare providers should not be routinely required to use more than one vial to administer a typical single dose of the drug product.
Multiple-dose vials should contain no more than 30 mL of drug product expect under specific circumstances.

In embodiments of the present invention, modular systems “routinely” require the use of more than one vial to prepare or administer a typical single dose of the drug product to a patient or animal. In one or more embodiments, routinely may mean at least 50% of the time it is required to use more than one vial to prepare or administer a typical single dose of the drug product.

In embodiments of the present invention, the dosing and assembly involves the preparation/assembly of customized amounts of medicaments in a ready-to-use/ready-to-assembly format. In embodiments of the present invention, a combination of vessels with a plurality of ports and containers housing non-standard amounts of beneficial substances/medicaments provide for customization of a final assembled dose for a patient. In embodiments of the invention, multiple drugs and/or therapies can be administered to a patient via a vessel or modular construction of containers.

In embodiments of the invention, the vessel and containers include decontamination devices and/or interfaces. In embodiments of the invention, the decontamination devices and interfaces include external, internal, moveable wiper, and static wiper decontamination devices.

In embodiments of the invention, the decontamination devices may be attached to a port or abut a wall of container. In embodiments of the invention, the decontamination devices are integrally attached to the vessel and or containers. In embodiments of the invention, the decontamination devices are in a unitary structure or arrangement with the vessel or container.

In embodiments of the invention, the entry ports include engagement mechanisms abutting the wall of the vessel or container. In embodiments of the invention, the entry ports include engagement mechanisms surface mounted to the wall of the vessel or container. In embodiments of the invention, the entry ports include engagement mechanisms flush mounted to the wall of the vessel or container. In embodiments of the invention, the entry ports include flush mounted engagement mechanisms. In embodiments of the invention, the entry ports include surface mounted engagement mechanisms. In embodiments, of the invention, the entry ports abutting a wall of the container.

In certain embodiments, the methods and systems are useful for pediatric, geriatric, and oncology patients. These patient populations require customized dosing based on age, weight, or body surface area.

In certain embodiments, the methods and systems involve preparation of oncology injectable drugs. In certain embodiments, the methods and systems involve dosing a chemotherapeutic/oncolytic/oncology medicament. In certain embodiments, the modular assembly obviates need to mix or intermix hazardous drug products.

In certain embodiments, the methods and systems involve double checking dosing regimens administered to patients. In certain embodiments, the dosing regimens have two double checks: (1) product double check of customized final amount; and (2) dosage double check of customized final amount.

In certain embodiments, the methods and systems involve providing amounts of drugs packaged by a manufacturer in a ready to assemble format in amounts less than about 10% of the amount in Table 1 Column A. For example, if Table 1 Column A amount is 100 milligrams (mg) then an amount less than about 10% of a column Table 1 Column A amount is 10 mg or less. “about” includes 10 mg″. For example, less than about 10% of 100 mg is any of the following 10 mg, 9 mg, 8 mg, 7 mg, 6 mg, 2 mg, 1 mg, 0.5 mg, 0.1 mg, etc. . . .

In certain embodiments, the non-standard amounts of drug involve amounts of drug that are not commonly provided by a drug manufacturer. FDA or another regulatory body would never in the current state of the art approve a non-standard amount that would require a typical user to use 2, 3, 4, 5, 6 or more vials/containers to prepare a single final dose amount for a patient.

In certain embodiments, the present invention provides for the packaging by a drug manufacturer or packager non-standard amounts of beneficial substance and/or drugs in ready to assemble containers. On the lower end of the spectrum commercially prepackaged non-standard amount may be: less than about 25% of a Table 1 Column A amount or Table 2 Column A amount, less than about 20% of a Table 1 Column A amount or a Table 2 Column A amount, less than about 10% of a Table 1 Column A amount or a Table 2 Column A amount, less than about 5% of a Table 1 Column A amount or a Table 2 Column A amount, less than about 3% of a Table 1 Column A or Table 2 Column A amount, less than about 2% of a Table 1 Column A or Table 2 Column A amount, and/or less than about 1% of a Table 1 Column A or Table 2 Column A amount.

In an aspect of the present invention, a vessel having a plurality of entry ports configured to engage a plurality of containers and a plurality of containers housing a beneficial substance and/or a medicament provides for a modular assembly of a final amount of a beneficial substance and/or a medicament.

In an aspect of the present invention, a vessel having a plurality of entry ports configured to engage a plurality of containers and at least one container housing a beneficial substance and/or medicament in a nonstandard amount and at least one container housing a beneficial substance and/or medicament in a standard amount provides for a modular assembly of a final amount of a beneficial substance and/or a medicament.

In an aspect of the present invention, a vessel having a plurality of entry ports configured to engage a plurality of containers and a plurality of containers housing a beneficial substance and/or medicament in a nonstandard amount provides for a modular assembly of a final amount of a beneficial substance and/or a medicament.

A further purpose of this invention is to provide a vessel and entry port that reduces the presence of non-purified air and/or air particles when mixing materials into a vessel. This invention focuses on modular systems and systems to connect dosing regimens to vessel to administer non-standard final amounts to patients.

In a certain aspect of the present invention, the present invention provides a manifold having a plurality of openings or ports configured to connect to a plurality of containers; and a plurality of containers housing a beneficial substance, wherein at least one of the plurality of containers houses a beneficial substance in a nonstandard amount. In one or more embodiments, the opening or ports of the manifold are configured to connect/couple with a plurality of connectors which in turn couple with a plurality of containers housing a beneficial substance. In certain embodiments, at least one of the containers housing a beneficial substance in a nonstandard amount.

In a certain aspect of the present invention, the present invention provides a connector with a plurality of openings or ports configured to connect to at least two containers; and a plurality of containers. In one or more embodiments, one of the at least two containers houses a beneficial substance in a nonstandard amount.

In another aspect, the present invention provides a system for the assembly of a beneficial substance, the system comprising: a first container having at least one entry port configured to engage a second container; a second container having at least one entry ports and at least one exit port, wherein the at least one entry port is configured to engage a third container and the at least one exit port is configured to engage the first container; at least a third container having at least one exit port; wherein upon engagement of the first container to the second container, and upon engagement of the second container to the third container, the contents of the third container flows into the second container and flows-through along with the contents of the second container into the first container. In one or more embodiments, the system for assembling the beneficial substance is modular by design. In one or more embodiments, a fourth container, a fifth container, or six or more containers may be provided by the system allowing a user of the system to modularly assemble any final amount of a beneficial substance and/or medicament.

In certain embodiments, the second container houses a beneficial substance, wherein the beneficial substance from the third container flows-through the second container and into the first container. In one or more embodiments, the modular system provides beneficial substances and/or medicaments in commercially packaged non-standard amounts. In one or more embodiments, commercially packaged means prepackaged. In one or more embodiments, commercially packaged means packaged by a drug/pharmaceutical manufacturer.

Referring to the figures:

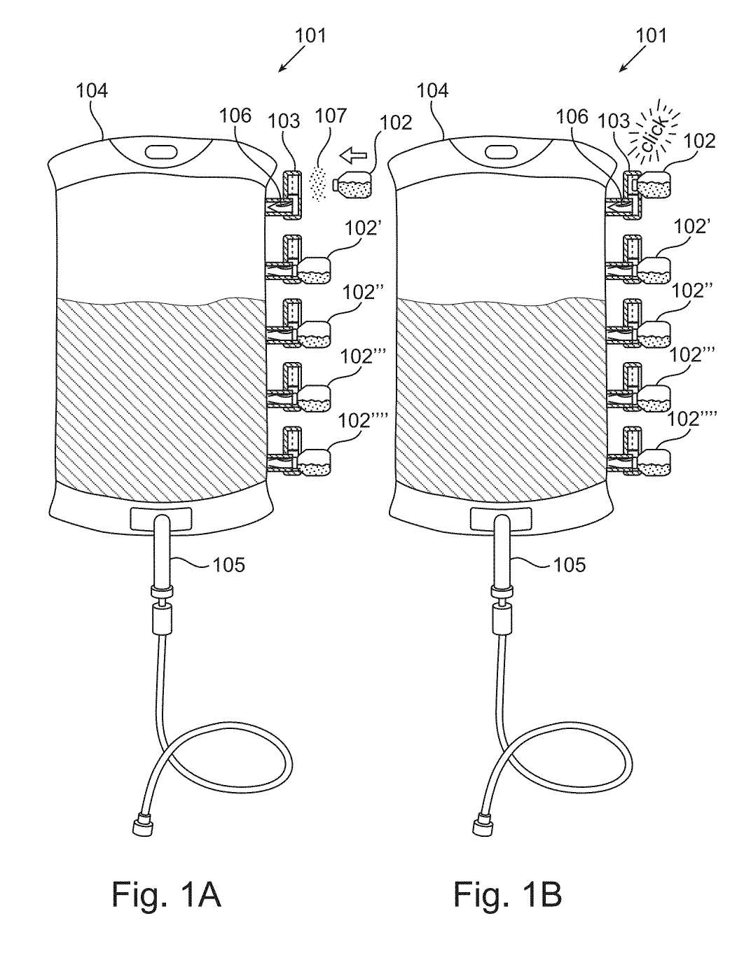

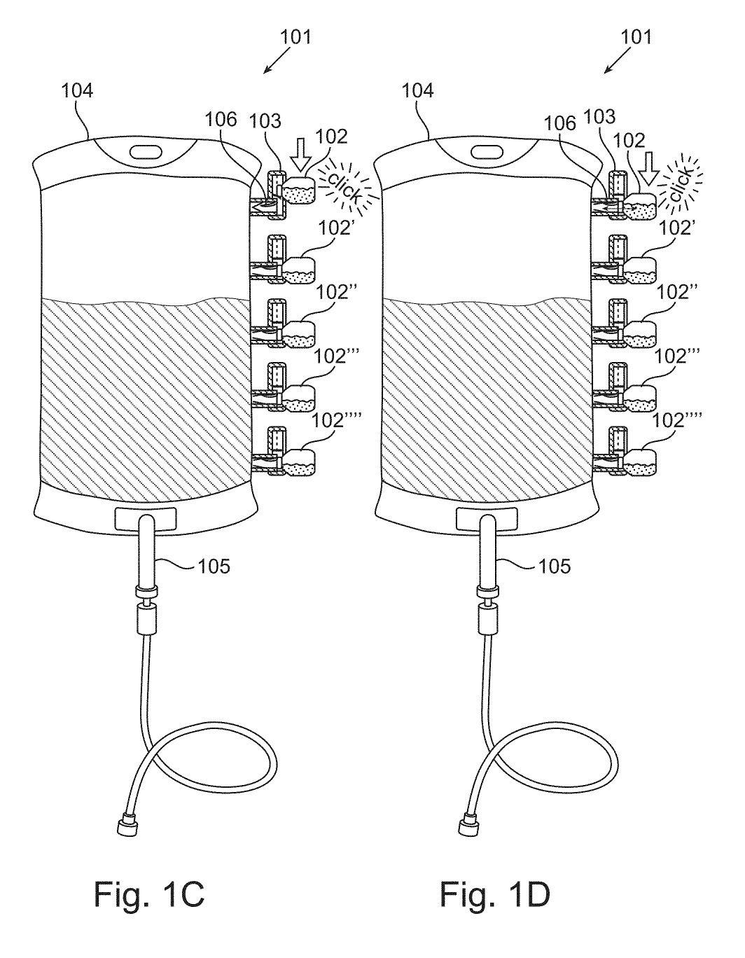

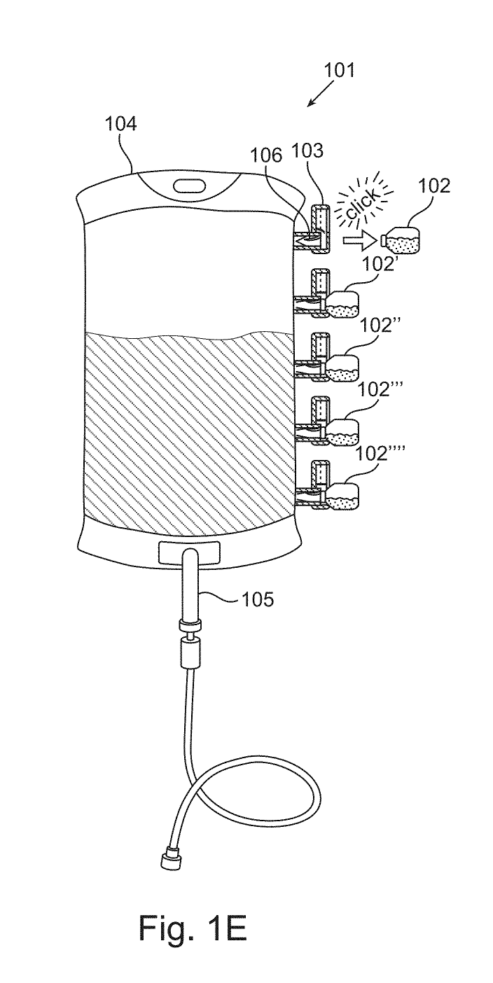

FIGS. 1A-1D are schematic front cut view illustrations presenting an exemplary system 101 which includes an IV bag 104 having a plurality of entry ports 103 and an exit port 105, wherein a plurality of containers 102 are received and engaged by the plurality of entry ports 103 to transfer a dose from the plurality of containers to the IV bag, according to some embodiments of the invention.

As shown in FIG. 1A, contaminants 107 are present between container 102 and entry port 103. In FIG. 1B, container 102 is engaged with entry port 103 and a “click” sound is made. Once the click sound is made, the container 102 is able to slide from a first compartment in the entry port 103 to a second compartment in the entry port 103 as shown in FIG. 1C. The contaminants 107 are not able to enter the second compartment as a wiping member is provided that removes the contaminants from the surface of container 102. In one or more embodiments, the “click” sound may be any other sound that alerts the user that container 102 is engaged to port 103 of IV bag 104.

FIG. 1D shows the transfer of dose from the container 102 through the entry port 103 into the IV bag 104 through which is a contaminant-free fluid passageway. In certain embodiments, a piercing member 106 is provided, such that upon movement of the container 102 from the first compartment to the second compartment, the piercing member 106 pierces the cap from the container 102, and allows the dose to pass through the contaminant-free fluid passageway, as shown in FIG. 1D. After the dose is transferred to the IV bag 104, FIG. 1E shows the removal of the container 102 from the entry port 103. In certain embodiments, the entry port 103 has a one way valve, so that air and contaminants 107 are not able to enter the IV bag 104. In one or more embodiments, entry port 103 is a decontamination device. In one or more embodiments, IV bag 104 is a container. In one or more embodiments, the container 104 is a bottle. In one or more embodiments, the interior of container 104 has a pressure less than ambient air pressure, ambient air pressure at sea level, and/or atmospheric pressure. In one or more embodiments, container 104 has an interior pressure less than the pressure of container 102. In one or more embodiments, container 104 has an interior pressure less than the pressure of the plurality of containers 102 that connect/couple with ports 103 of container 104. In one or more embodiments, the decontamination device (103) has a wiping member disposed within the decontamination device 103. In one or more embodiments, the decontamination device 103 has a rail or hinge mechanism. In one or more embodiments, the decontamination device 103 has an engagement mechanism configured to engage container 102. In one or more embodiments, the container 104 has a plurality of decontamination devices 103. In one or more embodiments, the container 104 has a plurality of decontamination devices 103 with a plurality of wiping member disposed within the decontamination devices 103. In one or more embodiments, the decontamination devices 103 are integrally manufactured with the container. In one or more embodiments, the container 104 is a bag 104. In one or more embodiments, the container 104 is a bottle. In one or more embodiments, the container 104 has a plurality of decontamination devices 103.

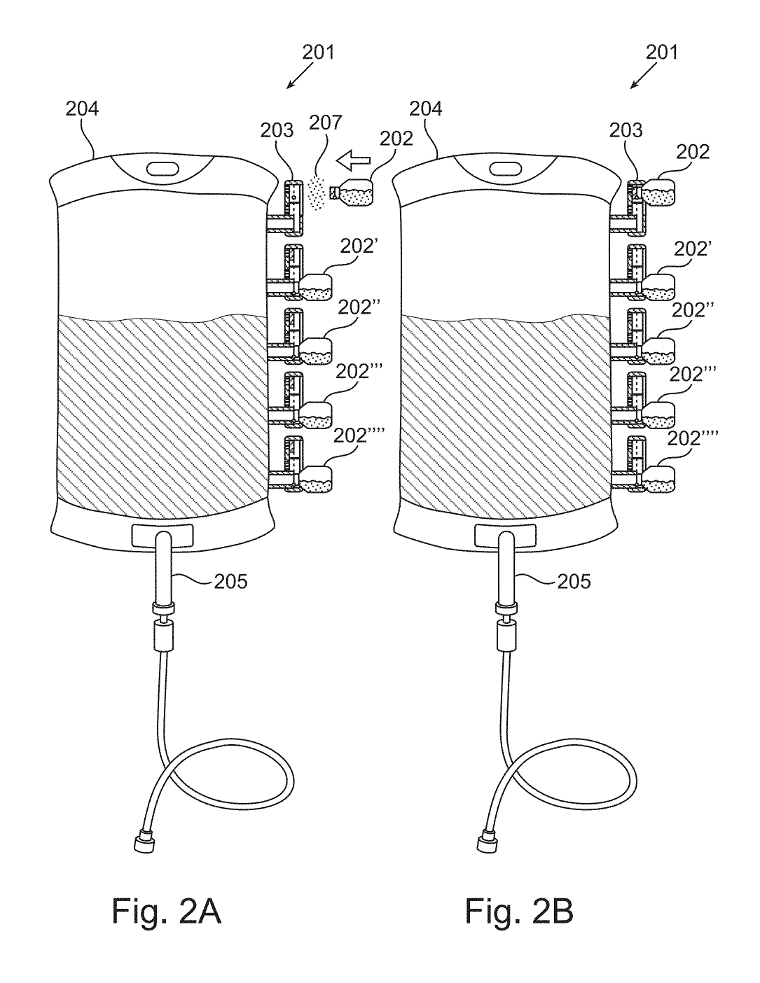

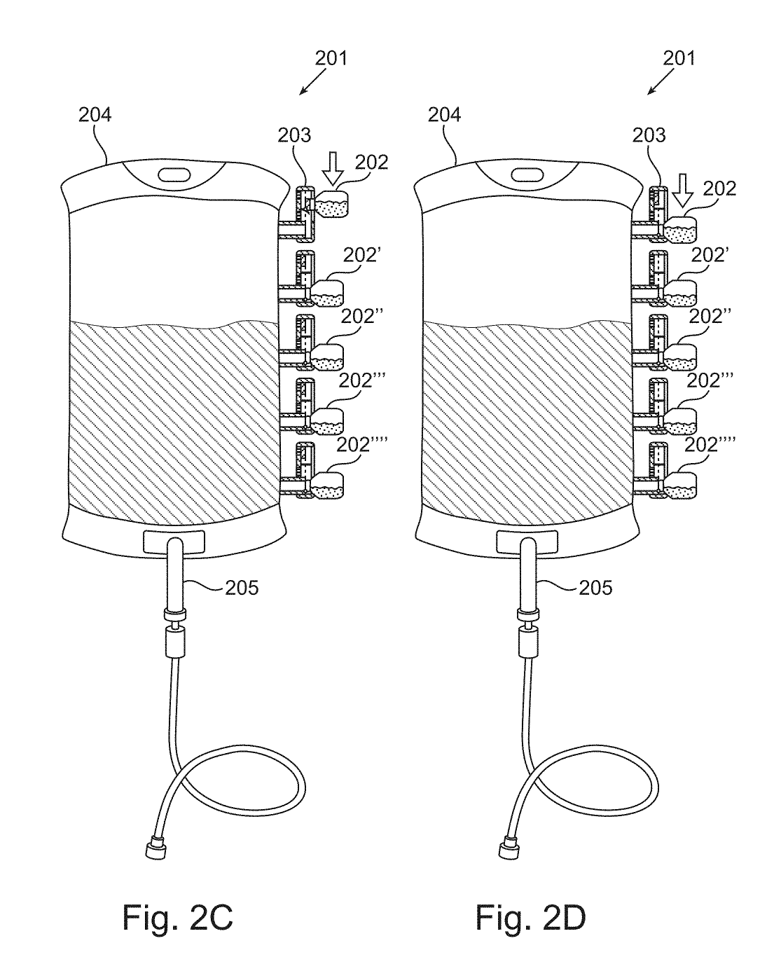

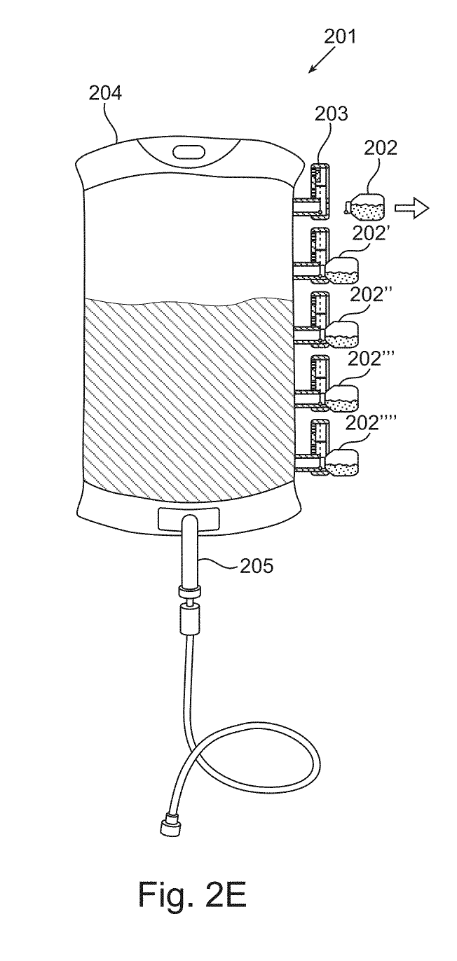

FIGS. 2A-2E are schematic front cut view illustrations presenting a further exemplary system 201 which includes an IV bag 204 having a plurality of entry ports 203 and an exit port 205, wherein a plurality of containers 202 are received and engaged by the plurality of entry ports 203 to transfer a dose from the plurality of containers 202 to the IV bag 204, according to some embodiments of the invention.

As shown in FIG. 2A, contaminants 207 are present between container 202 and entry port 203. In FIG. 2B, container 202 is engaged with entry port 203. The container 202 is able to slide from a first compartment in the entry port 203 to a second compartment in the entry port 203 as shown in FIG. 2C. The contaminants 207 are not able to enter the second compartment as a wiping member is provided that removes the contaminants from the surface of container 202.

FIG. 2D shows the transfer of dose from the container 202 through the entry port 203 into the IV bag 204 through which is a contaminant-free fluid passageway. After the dose is transferred, FIG. 2E shows the removal/detachment of the container 202 from the entry port 203. In certain embodiments, the entry port 203 has a one way valve (not shown), so that air and contaminants 207 are not able to enter the IV bag 204. In one or more embodiments, the plurality of entry ports 203 are decontamination devices. In one or more embodiments, the decontamination devices may be three, four or five compartment decontamination devices. In one or more embodiments, the decontamination devices have a plurality of internally separated compartments. In one or more embodiments, at least one of the compartments of the decontamination devices houses a sterilizing and/or disinfecting substance.

In one or more embodiments, containers 202 house a beneficial substance and/or a medicament. In one or more embodiments, the beneficial substance and/or medicament is in an nonstandard amount. In one or more embodiments, a plurality of the containers houses a beneficial substance in a nonstandard amount. In one or more embodiments, at least one of the containers 202 houses a beneficial substance/medicament in a standard amount and at least one of the container 202 houses a beneficial substance/medicament in a nonstandard amount.

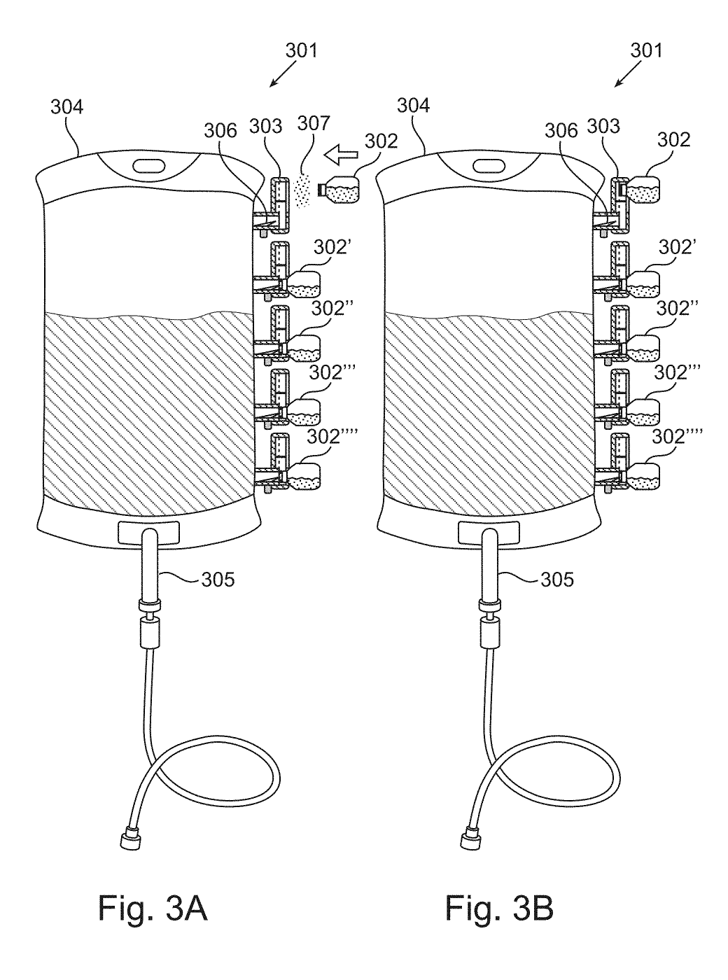

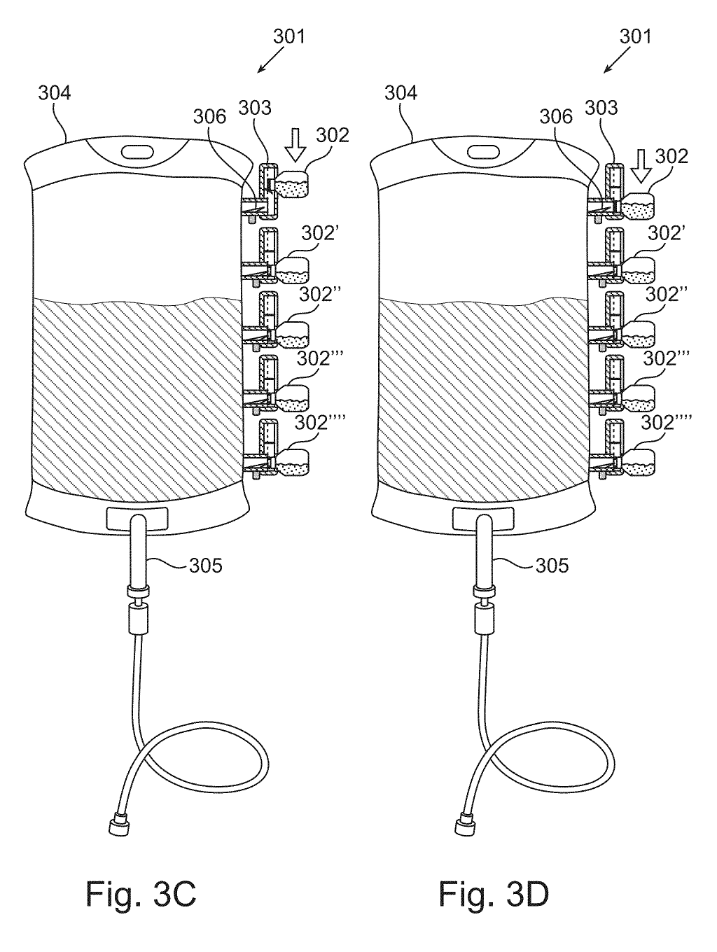

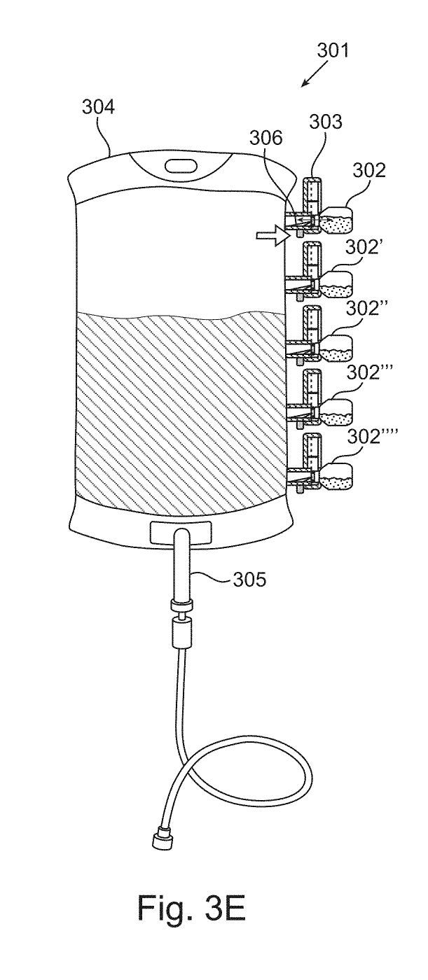

FIGS. 3A-3E are schematic front cut view illustrations presenting a further exemplary system which includes an IV bag 304 having a plurality of entry ports 303 and an exit port 305, wherein a plurality of containers 302 are received and engaged by the plurality of entry ports 303 to transfer a dose from the plurality of containers 302 to the IV bag 304, according to some embodiments of the invention.

As shown in FIG. 3A, contaminants 307 are present between container 302 and entry port 303. In FIG. 3B, container 302 is engaged with entry port 303. The container 302 is able to slide from a first compartment in the entry port 303 to a second compartment in the entry port 303 as shown in FIG. 3C. The contaminants 307 are not able to enter the second compartment as a wiping member is provided that removes the contaminants from the surface of container 302.

FIG. 3D shows the transfer of dose from the container 302 through the entry port 303 into the IV bag 304 through which is a contaminant-free fluid passageway. In certain embodiments, a piercing member 306 is provided, such that upon movement of the container 302 from the first compartment to the second compartment, the piercing member 306 pierces the cap from the container 302 and allows the dose to pass through the contaminant-free fluid passageway, as shown in FIG. 3E. In one or more embodiments, the movement of container 302 within the port 303 actuates and/or causes the piercing member 306 to pierce a surface of container 302. FIG. 3E shows how a user must manually push the container 302 towards the IV bag 304, or optionally how a user must manually push IV bag 304 towards container 302, thus causing the piercing member 306 to pierce the cap and/or seal of container 302. In this manner, the dose or amount in container 302 is able to pass through the contaminant-free fluid passageway. In certain embodiments, the entry port 303 has a one way valve (not shown), so that air and contaminants 307 are not able to enter the IV bag 304. In certain embodiments, entry port 303 is a decontamination device. In certain embodiments, the entry port 303 and/or decontamination device 303 is integrally manufactured with and/or forms a unitary structure with IV bag 304.

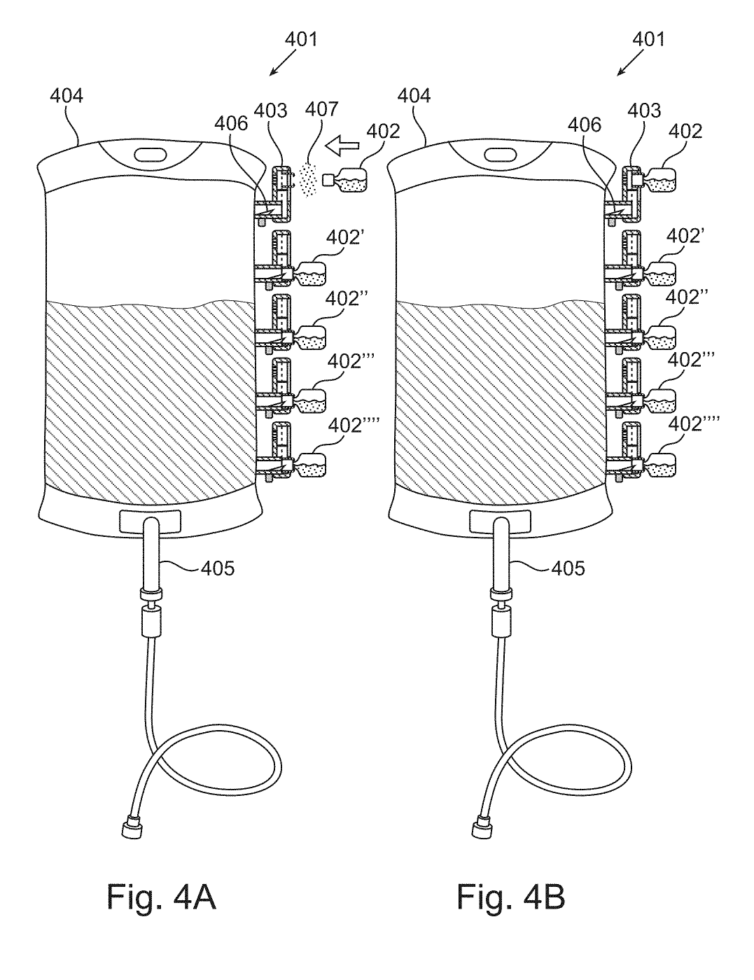

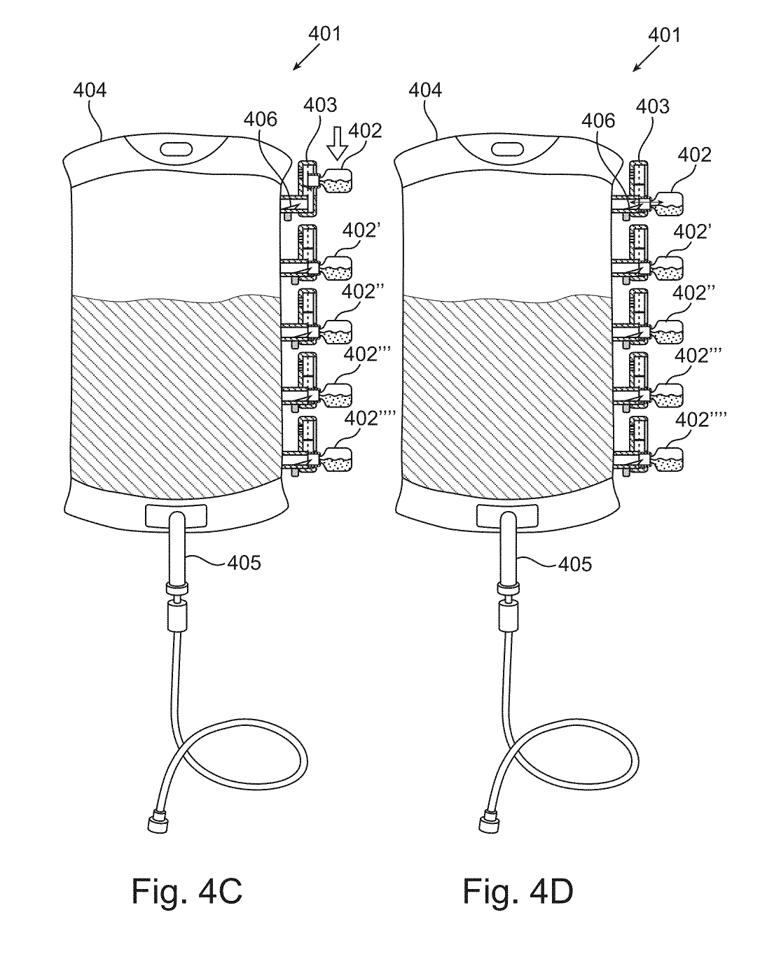

FIGS. 4A-4D are schematic front cut view illustrations presenting a further exemplary system 401 which includes an IV bag 404 having a plurality of entry ports 403 and an exit port 405, wherein a plurality of containers 402 are received and engaged by the plurality of entry ports 403 to transfer a dose from the plurality of containers to the IV bag, according to some embodiments of the invention.

As shown in FIG. 4A, contaminants 407 are present between container 402 and entry port 403. In FIG. 4B, container 402 is engaged with entry port 403 via a clamping member on the entry port 403. The container 402 is able to slide from a first compartment in the entry port 403 to a second compartment in the entry port 403 as shown in FIG. 4C. The contaminants 407 are not able to enter the second compartment as a wiping member is provided that removes the contaminants from the surface of container 402.

FIG. 4D shows the transfer of dose from the container 402 through the entry port 403 into the IV bag 404 through which is a contaminant-free fluid passageway. In certain embodiments, a piercing member 406 is provided, such that upon movement of the container 402 from the first compartment to the second compartment, the piercing member 406 pierces the cap from the container 402, and allows the dose to pass through the contaminant-free fluid passageway. In certain embodiments, the entry port 403 has a one way valve, so that air and contaminants 407 are not able to enter the IV bag 404. Optionally, piercing member 406 may pierce a surface of IV bag 404. IV bag 404 is used arbitrarily in all the figures and may be interchanged with any other vessel such as a bottle, a container, a manifold, a connector with multiple openings/ports and/or a syringe.

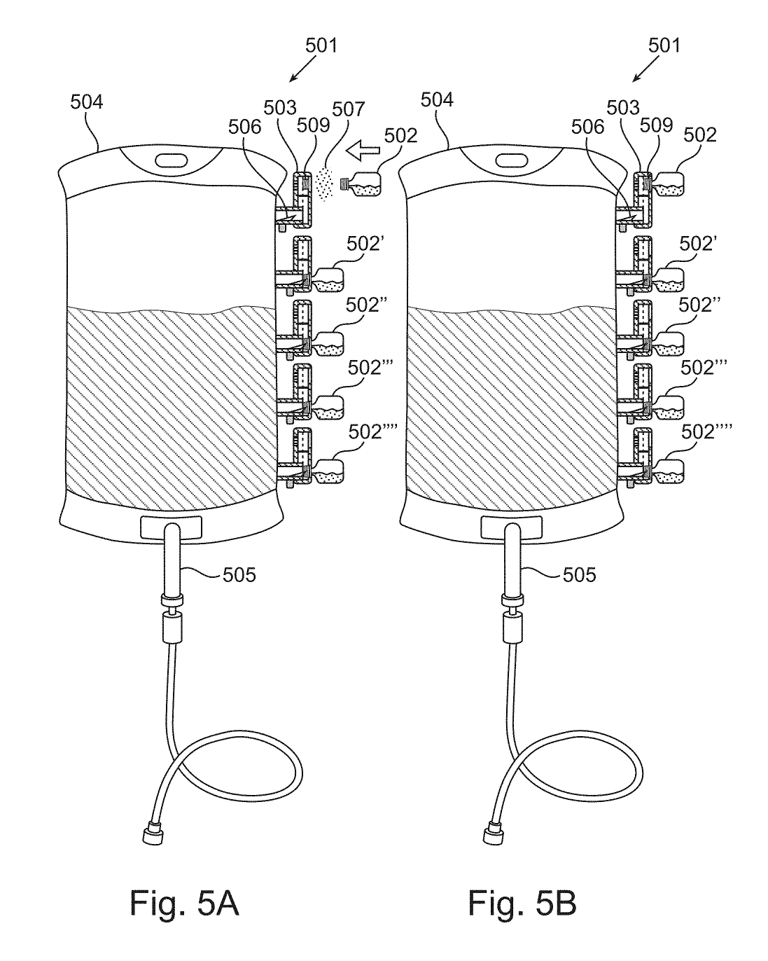

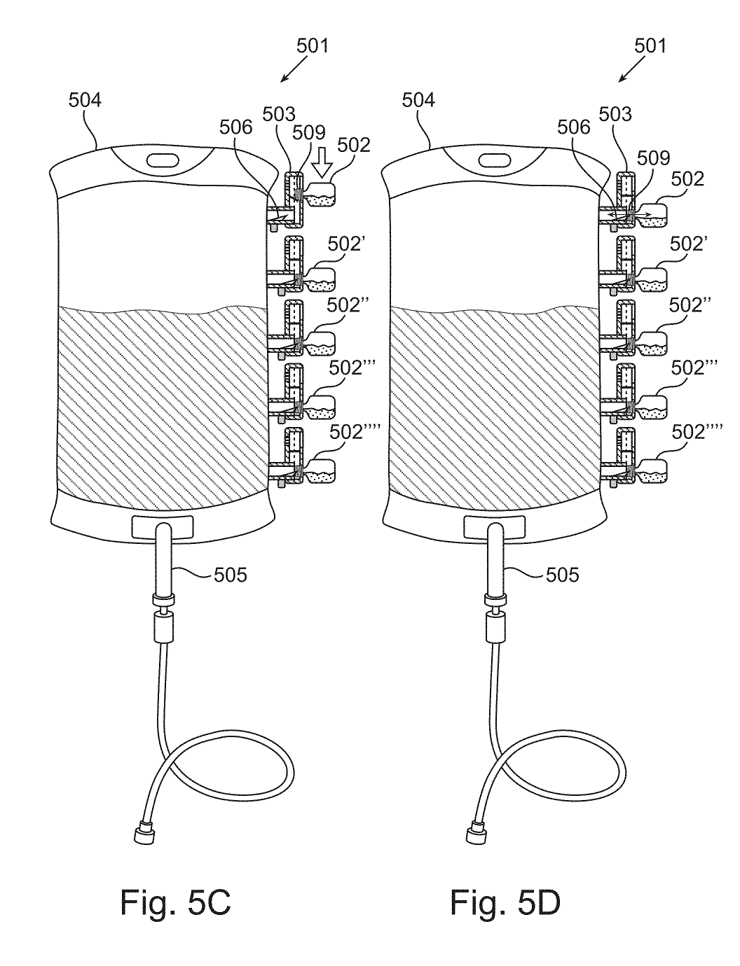

FIGS. 5A-5D are schematic front cut view illustrations presenting a further exemplary system which includes a IV bag 504 having a plurality of entry ports 503 and an exit port 505, wherein a plurality of containers 502 are received and engaged by the plurality of entry ports 503 to transfer a dose from the plurality of containers 502 to the IV bag 504, according to some embodiments of the invention.

As shown in FIG. 5A, contaminants 507 are present between container 502 and entry port 503. In FIG. 5B, container 502 is engaged with entry port 503 via a thread 509 on or in the entry port 503. The container 502 is able to slide from a first compartment in the entry port 503 to a second compartment in the entry port 503 as shown in FIG. 5C. The contaminants 507 are not able to enter the second compartment as a wiping member is provided that removes the contaminants from the surface of container 502. The thread 509 may be a luer, a luer-lock, a luer-slip or a smartside.

FIG. 5D shows the transfer of dose from the container 502 through the entry port 503 into the IV bag 504 through which is a contaminant-free fluid passageway. In certain embodiments, a piercing member 506 is provided, such that upon movement of the container 502 from the first compartment to the second compartment, the piercing member 506 pierces the cap from the container 502, and allows the dose to pass through the contaminant-free fluid passageway. In certain embodiments, the entry port 503 has a one way valve, so that air and contaminants 507 are not able to enter the IV bag 504.

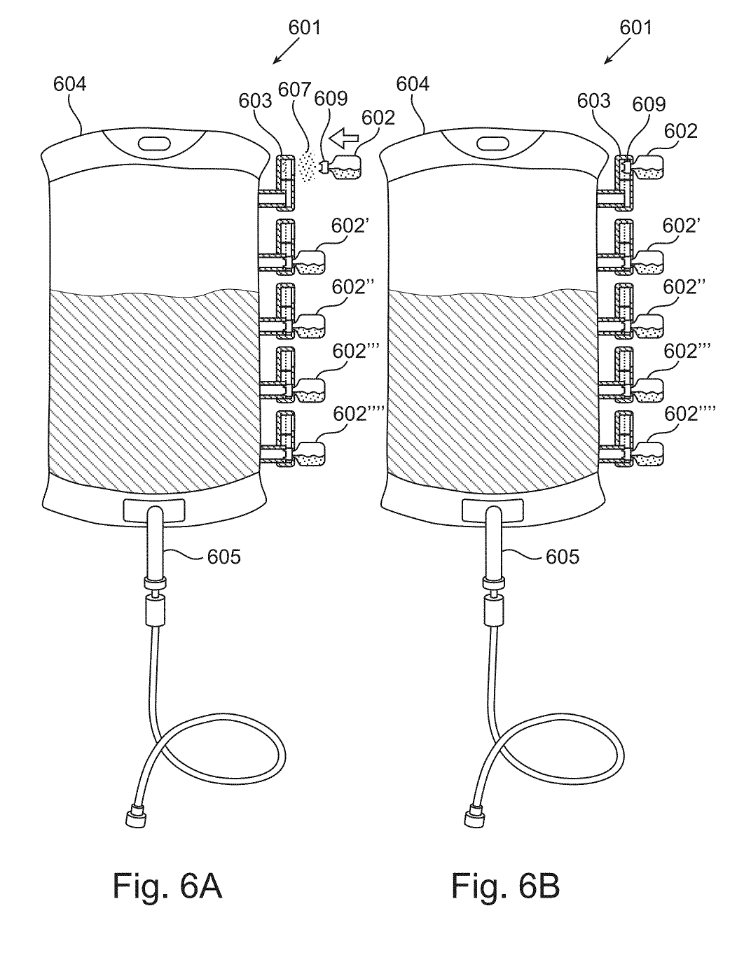

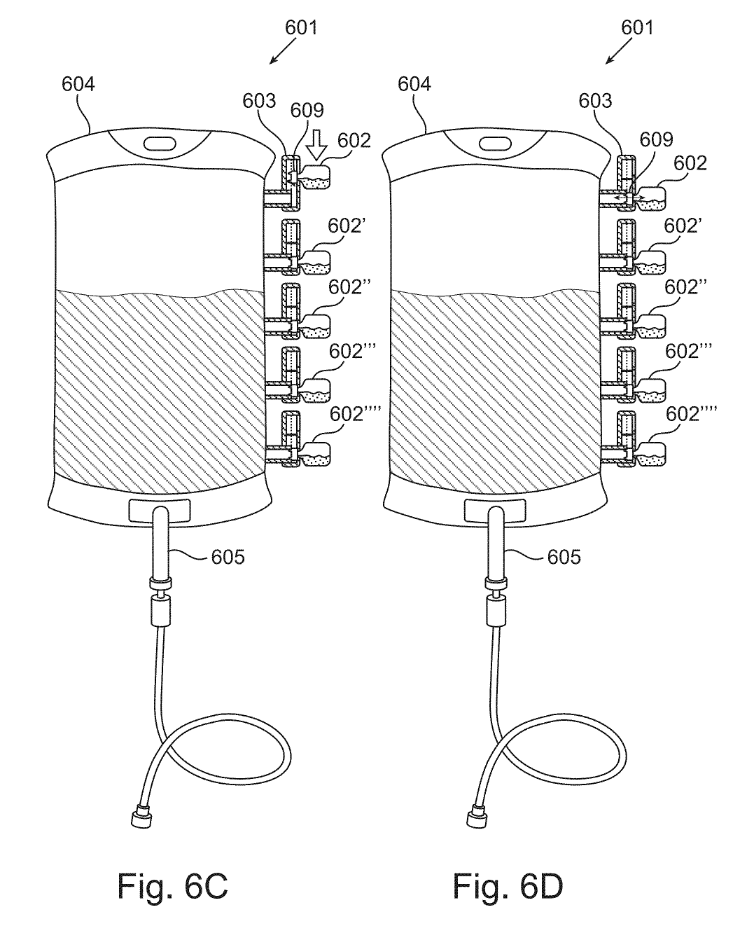

FIGS. 6A-6D are schematic front cut view illustrations presenting a further exemplary system 601 which includes an IV bag 604 having a plurality of entry ports 603 and an exit port 605, wherein a plurality of containers 602 are received and engaged by the plurality of entry ports 603 to transfer a dose from the plurality of containers 602 to the IV bag 604, according to some embodiments of the invention.

As shown in FIG. 6A, contaminants 607 are present between container 602 and entry port 603. In FIG. 6B, container 602 is engaged with entry port 603 via ratchet teeth 609 on the container 602. The container 602 is able to slide from a first compartment in the entry port 603 to a second compartment in the entry port 603 as shown in FIG. 6C. The contaminants 607 are not able to enter the second compartment as a wiping member is provided that removes the contaminants from the surface of container 602. For all of FIGS. 6A-6D, the entry ports 603 may be decontamination Devices. Optionally, entry ports 603 and/or decontamination devices 603 may be flush mounted or surface mounted to IV bag 604. Optionally, entry ports and/or decontamination device 603 may abut a wall of IV bag 604. Optionally, entry and/or decontamination devices 603 may abut a side wall of IV bag 604. IV bag 604 is used arbitrarily and may be any vessel such as a container, a bottle, a vial, a cartridge, or a syringe.

FIG. 6D shows the transfer of dose from the container 602 through the entry port 603 into the IV bag 604 through which is a contaminant-free fluid passageway. In certain embodiments, the entry port 603 has a one way valve, so that air and contaminants 607 are not able to enter the IV bag 604.

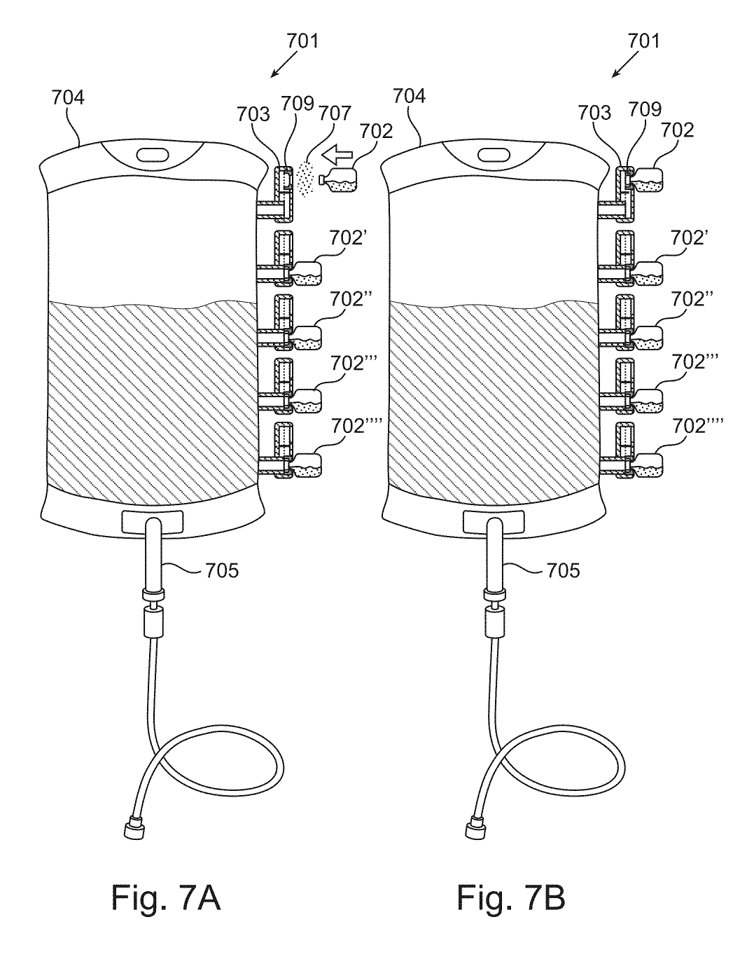

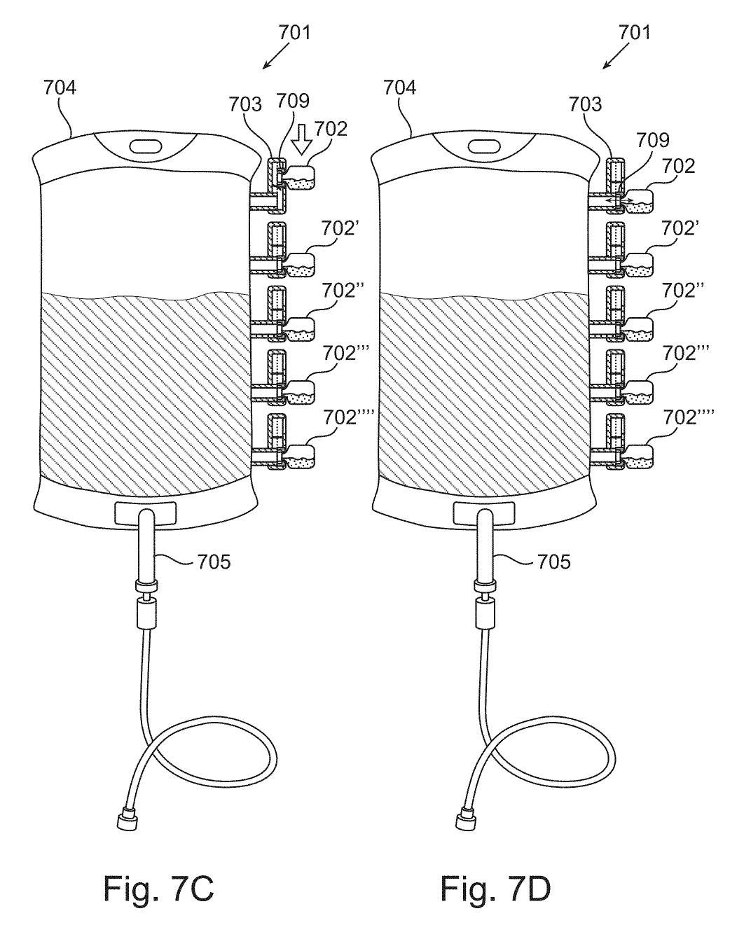

FIGS. 7A-7D are schematic front cut view illustrations presenting a further exemplary system 701 which includes an IV bag 704 having a plurality of entry ports 703 and an exit port 705, wherein a plurality of containers 702 are received and engaged by the plurality of entry ports 703 to transfer a dose from the plurality of containers 702 to the IV bag 704, according to some embodiments of the invention.

As shown in FIG. 7A, contaminants 707 are present between container 702 and entry port 703. In FIG. 7B, container 702 is engaged with entry port 703. A circumferential wiper 709 is provided within the entry port 703 which wipes the sides of the ports of container 702. In one or more embodiments, the circumferential wiper 709 may be made of an elastomeric and/or rubber material. In one or more embodiments, the circumferential wiper 709 may be cover by a sterilizing and/or disinfecting agent. The container 702 is able to slide from a first compartment in the entry port 703 to a second compartment in the entry port 703 as shown in FIG. 7C. The contaminants 707 are not able to enter the second compartment as a wiping member is provided that removes the contaminants from the surface of container 702.

FIG. 7D shows the transfer of dose from the container 702 through the entry port 703 into the IV bag 704 through which is a contaminant-free fluid passageway. In certain embodiments, the entry port 703 has a one way valve, so that air and contaminants 707 are not able to enter the IV bag 704.

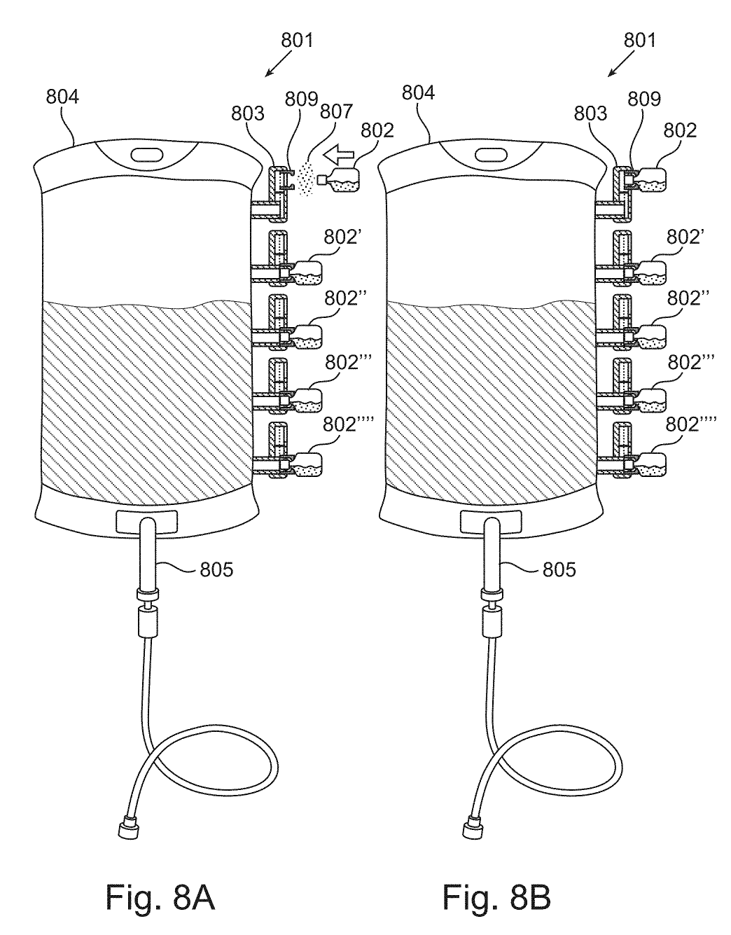

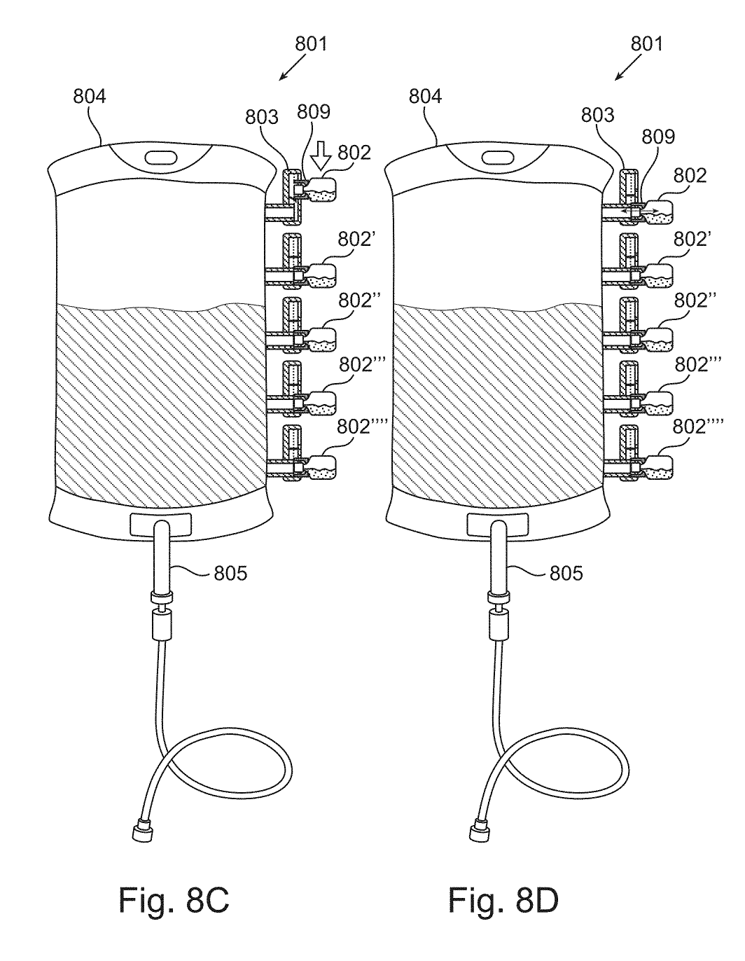

FIGS. 8A-8D are schematic front cut view illustrations presenting a further exemplary system 801 which includes a IV bag 804 having a plurality of entry ports 803 and an exit port 805, wherein a plurality of containers 802 are received and engaged by the plurality of entry ports 803 to transfer a dose from the plurality of containers 802 to the IV bag 804, according to some embodiments of the invention.

As shown in FIG. 8A, contaminants 807 are present between container 802 and entry port 803. In FIG. 8B, container 802 is engaged with entry port 803 via a clasping member 809. The container 802 is able to slide from a first compartment in the entry port 803 to a second compartment in the entry port 803 as shown in FIG. 8C via a rail or hinge mechanism. The contaminants 807 are not able to enter the second compartment as a wiping member is provided within the ports 803 that removes the contaminants from the surface of container 802.

FIG. 8D shows the transfer of dose from the container 802 through the entry port 803 into the IV bag 804 through which is a contaminant-free fluid passageway. In certain embodiments, the entry port 803 has a one way valve, so that air and contaminants 807 are not able to enter the IV bag 804. The one way valve of entry port 803 may prevent backflow of a beneficial substance from IV bag 804 to containers 802. Optionally, containers 802 may have a valve, wherein the valve may be a one way valve.

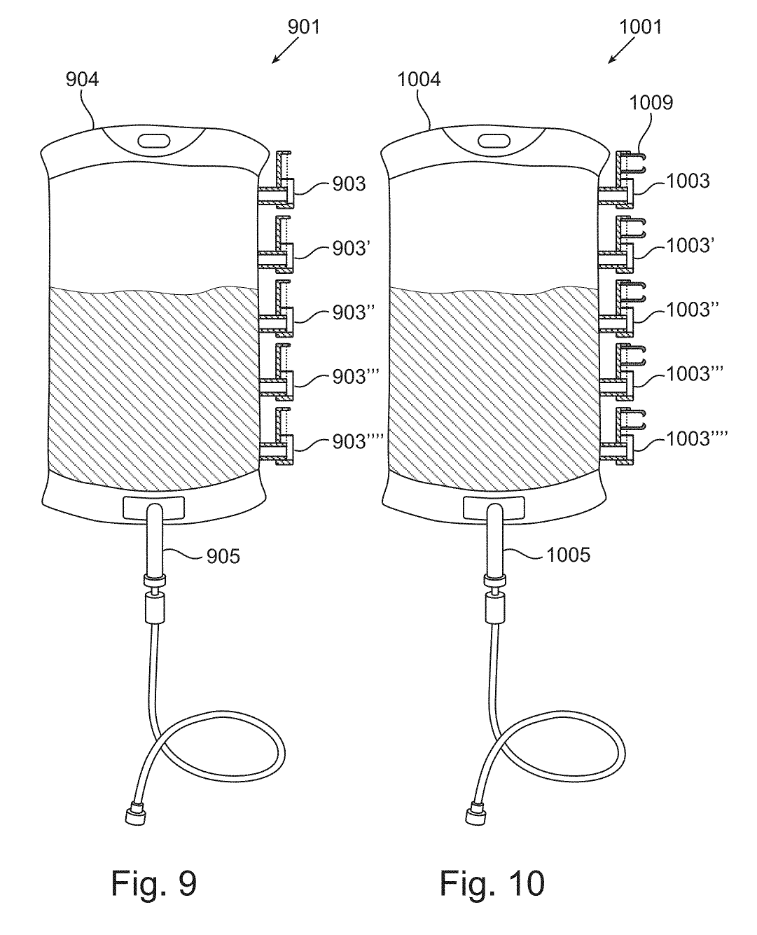

FIG. 9 is a schematic front cut view illustration presenting a further exemplary system 901 which includes a IV bag 904 having a plurality of entry ports 903 and an exit port 905, according to some embodiments of the invention. FIG. 9 shows a rail member such that a container is able to be received by the rail member and slide from a first position to a second position, which includes a contaminant-free fluid passageway that is hermetically sealed in order to transfer a dose into the IV bag 904.

FIG. 10 is a schematic front cut view illustration presenting a further exemplary system 1001 which includes a IV bag 1004 having a plurality of entry ports 1003 and an exit port 1005, according to some embodiments of the invention. FIG. 10 shows a clasping member 1009 and a rail member 1003 such that a container is able to be received by the rail member and slide from a first position to a second position, which includes a contaminant-free fluid passageway that is hermetically sealed in order to transfer a dose into the IV bag 1004. The IV bag 1004 has a plurality of rail members 903.

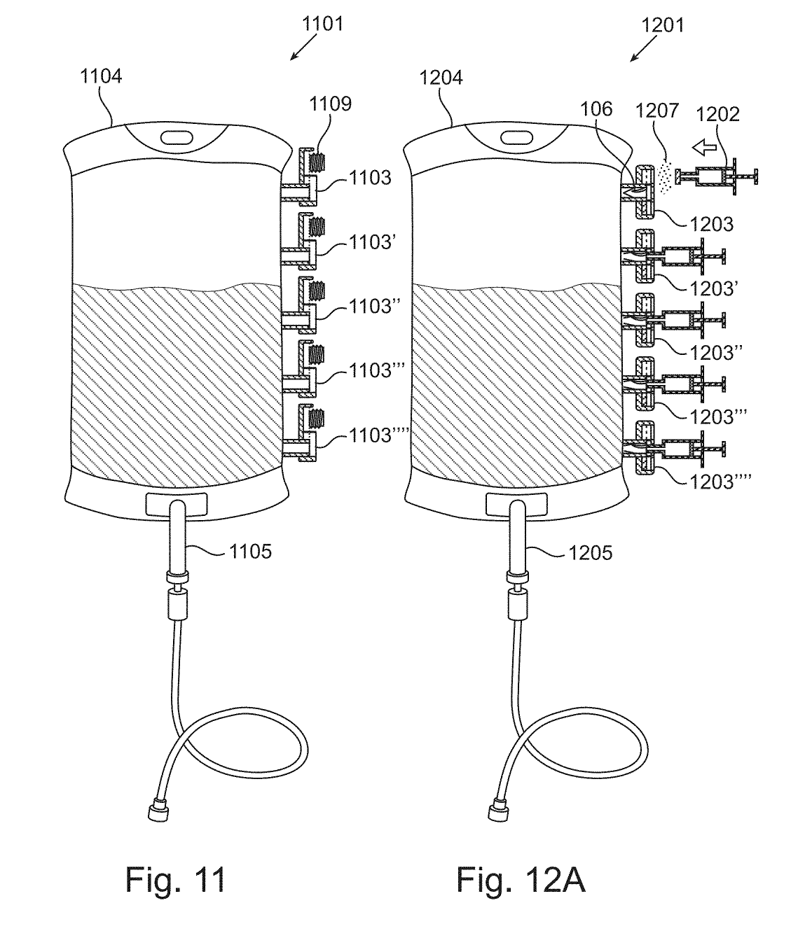

FIG. 11 is a schematic front cut view illustration presenting a further exemplary system 1101 which includes IV bag 1104 having a plurality of entry ports 1003 and an exit port 1105, according to some embodiments of the invention. FIG. 11 shows a thread 1109 such that a container is able to be received by the thread and slide from a first position to a second position, which includes a contaminant-free fluid passageway that is hermetically sealed in order to transfer a dose into the IV bag 1104.

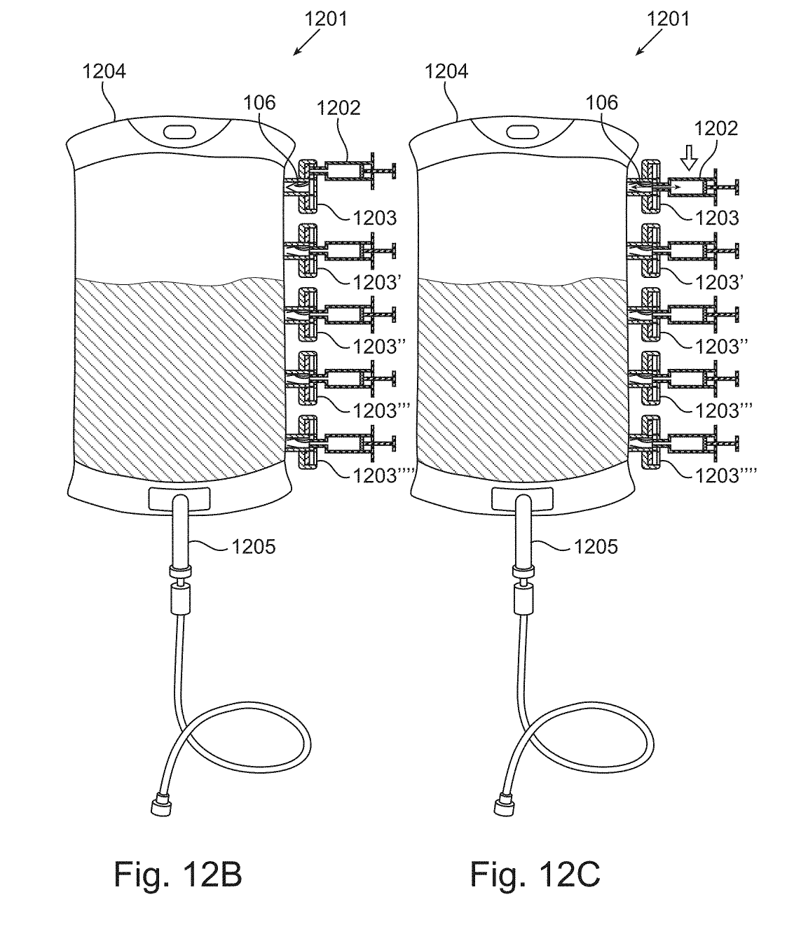

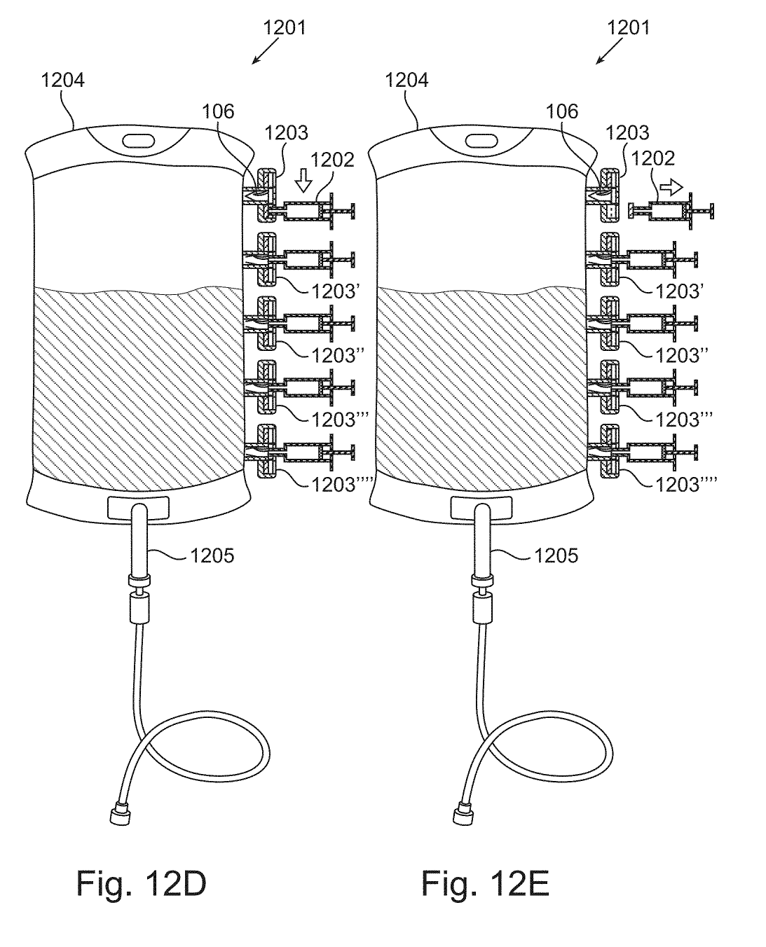

FIGS. 12A-12E are schematic front cut view illustrations presenting a further exemplary system 1201 which includes a IV bag 1204 having a plurality of entry ports 1203 and an exit port 1205, wherein a plurality of syringe elements 1202 are received and engaged by the plurality of entry ports 1203 to transfer a dose from the plurality of syringe elements 1202 to the IV bag 1204, according to some embodiments of the invention. Optionally, the syringe elements 1202 may be containers with an expulsion member. In one or more embodiments, at least one of syringe elements 1202 may house a beneficial substance and/or medicament in a nonstandard amount. In one or more embodiments, a plurality of syringe elements 1202 may house a beneficial substance and/or medicament in a nonstandard amount.

As shown in FIG. 12A, contaminants 1207 are present between syringe element 1202 and entry port 1203. In FIG. 12B, syringe element 1202 is engaged with entry port 1203. The syringe element 1202 is able to slide from a first compartment in the entry port 1203 to a second compartment in the entry port 1203 as shown in FIG. 12C. The contaminants 1207 are not able to enter the second compartment as a wiping member is provided that removes the contaminants from the surface of syringe element 1202.

FIG. 12C shows the transfer of dose from the syringe element 1202 through the entry port 1203 into the IV bag 1204 through which is a contaminant-free fluid passageway in the second/middle compartment. After the dose is transferred, FIG. 12D shows the syringe element 1202 sliding to a third compartment whereby the syringe element 1202 is able to be removed from the entry port 1203. In certain embodiments, the entry port 1203 has a one way valve, so that air and contaminants 1207 are not able to enter the IV bag 1204. Optionally, entry ports 1203 may have actuators that move the piercing member 106 through a surface of at least one of IV bag 1204 and/or syringe element 1202. Optionally, entry ports 1203 may have a frangible seal in the middle compartment that breaks upon the application of pressure to the frangible seal, thus establishing a fluidic passageway between syringe elements 1202 and IV bag 1204.

In certain embodiments, the third compartment of entry ports 1203 have a resealing element, such that upon entering the third compartment, the distal end of the syringe element 1202 mates with the resealing element. Afterwards, the syringe element 1202 is removed from the entry port 1203. The mating of the syringe element 1202 and the resealing element disposed within entry ports 1203 occurs within the third compartment of entry ports 1203. In one or more embodiments, exit port 1205 may have a decontamination device attached, coupled, integrally attached, and/or form a unitary structure with IV bag 1204.

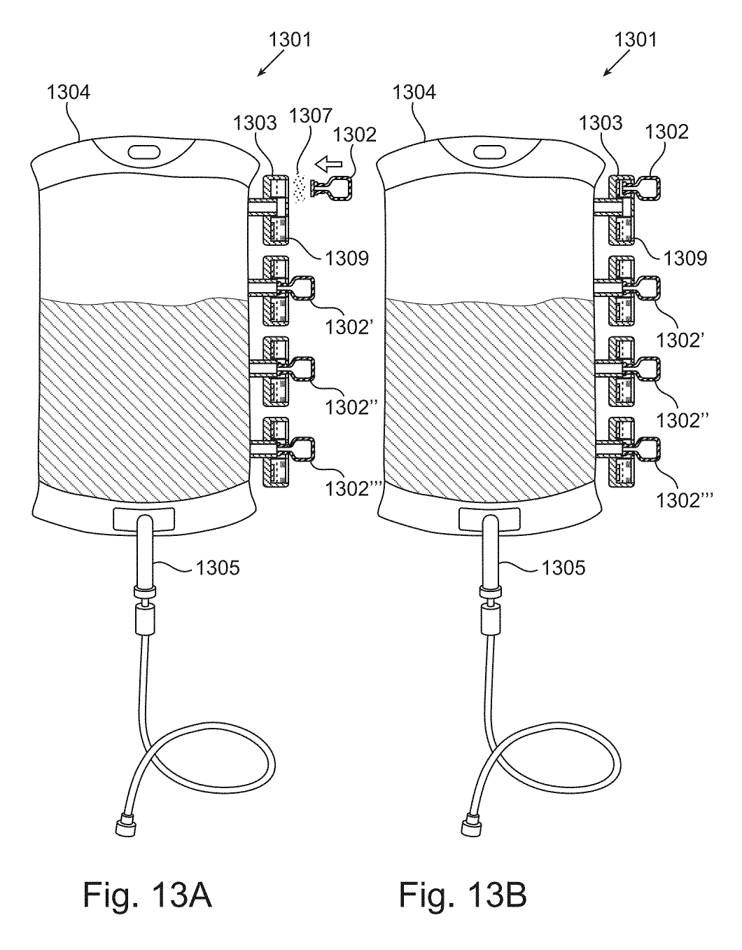

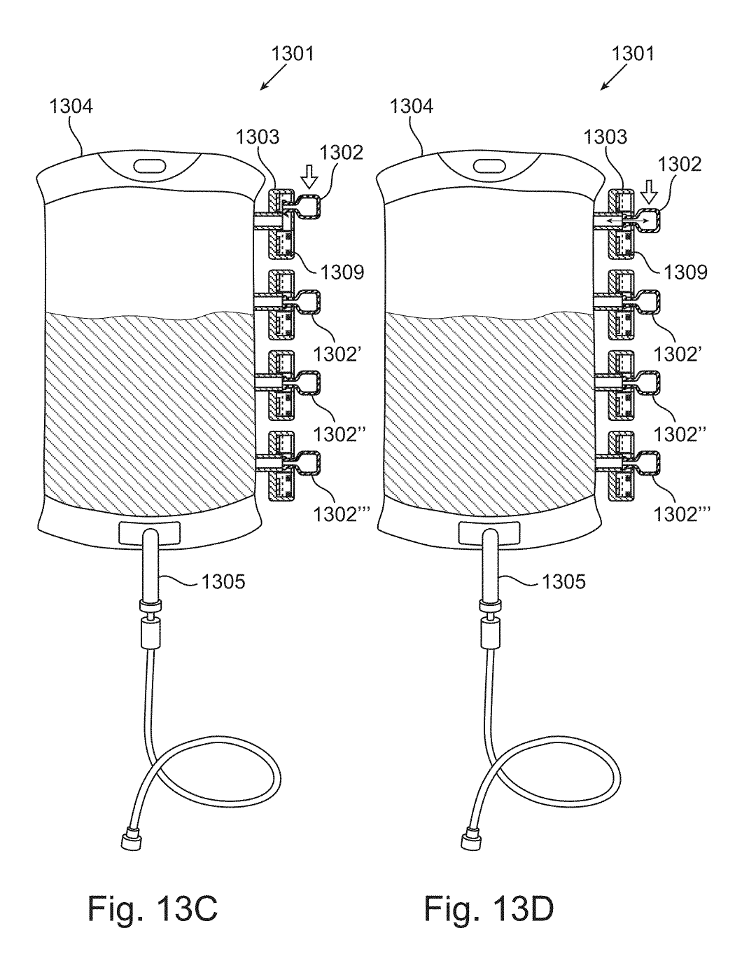

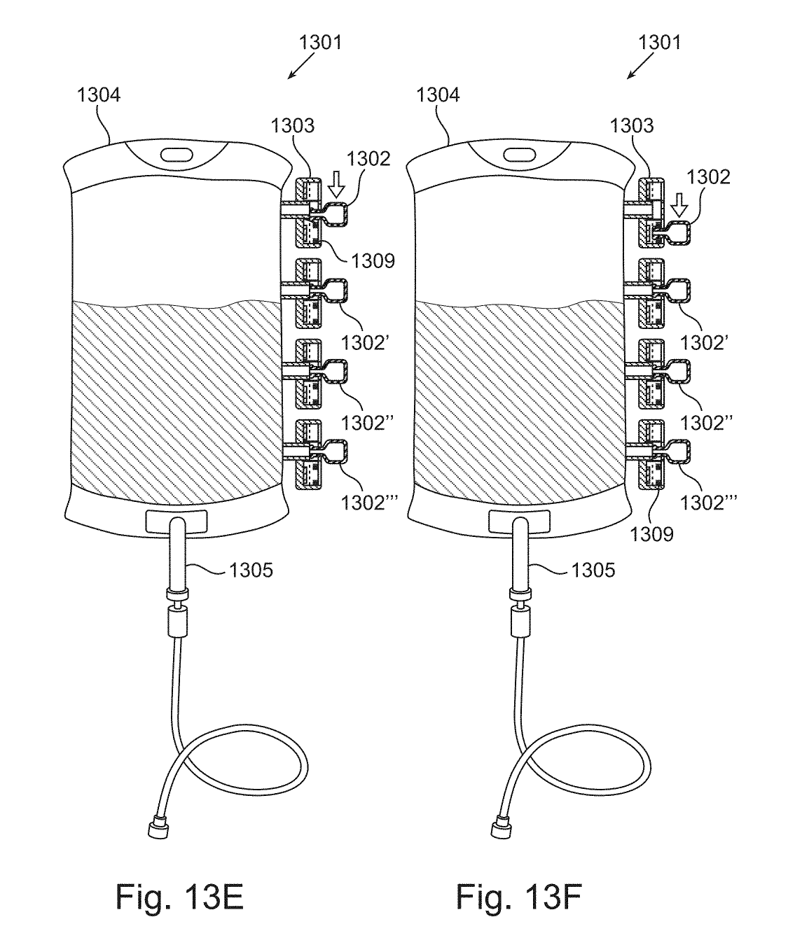

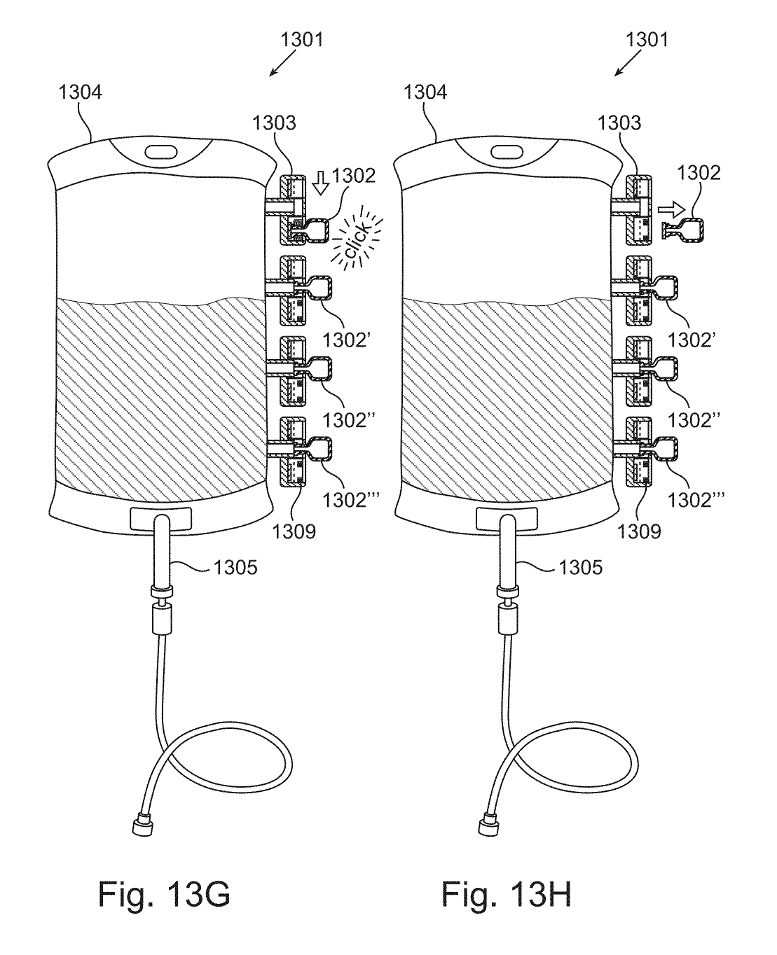

FIGS. 13A-13H are schematic front cut view illustrations presenting a further exemplary system 1301 which includes a IV bag 1304 having a plurality of entry ports 1303 and an exit port 1305, wherein a plurality of containers 1302 are received and engaged by the plurality of entry ports 1303 to transfer a dose from the plurality of containers 1302 to the IV bag 1304, according to some embodiments of the invention. As shown, container 1302 is a bag, a bottle or vial. It must be noted that any of entry ports 1303 may optionally be a four compartment or five compartment entry port.

As shown in FIG. 13A, contaminants 1307 are present between container 1302 and entry port 1303. In FIG. 13B, container 1302 is engaged with entry port 1303. The container 1302 is able to slide from a first compartment in the entry port 1303 to a second compartment in the entry port 1303 as shown in FIG. 13C. The contaminants 1307 are not able to enter the second compartment as a wiping member is provided that removes the contaminants from the surface of container 1302. Optionally, container 1302 may slide to a fourth compartment (not shown) and/or a fifth compartment (not shown) of entry port 1303.

FIG. 13D shows the transfer of dose from the container 1302 through the entry port 1303 into the IV bag 1304 through which is a contaminant-free fluid passageway in the second/middle compartment of entry port 1303. After the dose is transferred, FIGS. 13E-13F shows the container 1302 sliding to a third compartment whereby the container 1302 is able to be removed from the entry port 1303.

FIGS. 13G-13H show the container 1302 pressed down and/or pushed in until a “click sound” is made, whereby the seal between the container 1302 and the entry port 1303 is released. Afterwards, container 1302 is able to be removed from entry port 1303 by pulling or twisting the container 1302 away from entry port 1303 and IV bag 1304. Entry ports 1303 have a spring element 1309 which provide for movement of container 1302 within the third compartment of entry port 1303. Optionally, the spring element 1309 may be a rubber element, an elastomeric material, or any type of flexible material that allows for a surface or port of container 1302 to move within the third compartment of entry port 1303 allowing for the resealing of an aperture on a surface of container 1302 with a displaceable, sealing, and/or resealing member disposed within third compartment of port 1303. In one or more embodiments, the vessel/container 1304 has a plurality of sealing/resealing members disposed within entry ports 1303, wherein the entry ports may be decontamination devices. In one or more embodiments, entry ports 1303 may have four or five compartments. In one or more embodiments, entry ports 1303 may have a plurality of compartment. In one or more embodiments, entry ports 1303 may be decontamination devices having at least four compartments.

In certain embodiments, the third compartment of entry ports 1303 has a resealing element, such that upon entering the third compartment, the distal end (port) of the container 1302 mates with the resealing element. Afterwards, the container 1302 is removed from the entry port 1303. The resealing element disposed within the third compartment of entry ports 1303 may be a cap, a seal, a twist-on cap, a snap-on cap and combinations thereof.

In certain embodiments, container 1303 may be detached from the second compartment of entry port 1303.

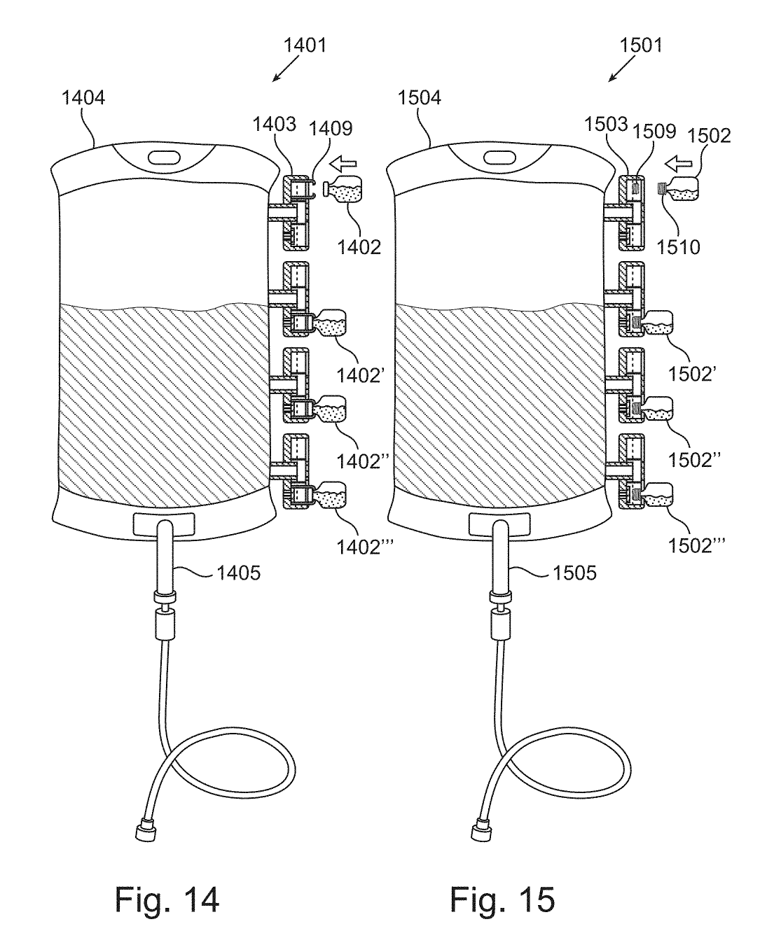

FIG. 14 is schematic front cut view illustrations presenting a further exemplary system 1401 which includes a IV bag 1404 having a plurality of entry ports 1403 and an exit port 1405, wherein a plurality of containers 1402 are received and engaged by the plurality of entry ports 1403 to transfer a dose from the plurality of containers 1402 to the IV bag 1404, according to some embodiments of the invention. FIG. 14 shows a clasping member 1409 and a rail member (dotted line) such that a container 1402 is able to be received by the clasping member 1409 and afterwards the clasping member along with the containers 1402 slide along the rail member (dotted line) and slide from a first position to a second position, which includes a contaminant-free fluid passageway that is hermetically sealed in order to transfer a dose into the IV bag 1404. Clasping member 1409 may be attached, integrally attached, and/or form a unitary structure with the rail member of entry ports 1403.

FIG. 15 is a schematic front cut view illustration presenting a further exemplary system 1501 which includes a IV bag 1504 having a plurality of entry ports 1503 and an exit port 1505, wherein a plurality of containers 1502 are received and engaged by the plurality of entry ports 1503 to transfer a dose from the plurality of containers 1502 to the IV bag 1504, according to some embodiments of the invention. FIG. 15 shows a thread member 1509 in the entry port and thread member 1510 on the container 1502 that engage and slide the container 1502 from a first position to a second position, which includes a contaminant-free fluid passageway that is hermetically sealed in order to transfer a dose into the IV bag 1504.

After the dose is transferred, the container 1502 slides into a third compartment whereby the container 1502 is able to be removed from the entry port 1503. Container 1502 is able to be removed from entry port 1503 by pulling or twisting or a combination of pulling and twisting the container 1502 away from entry port 1503 and IV bag 1504.

In certain embodiments, the third compartment has a resealing element, such that upon entering the third compartment, the distal end of the container 1502 mates with the resealing element. Afterwards, the container 1502 is removed from the entry port 1503.

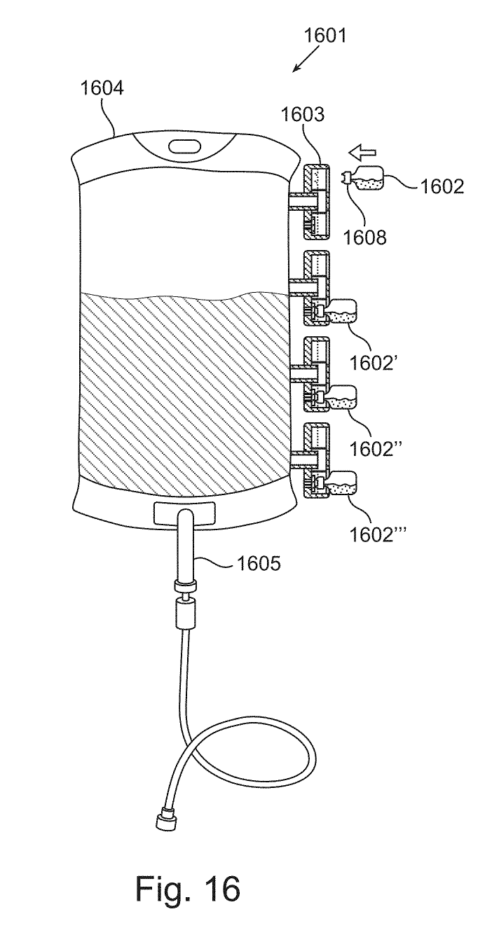

FIG. 16 is a schematic front cut view illustration presenting a further exemplary system 1601 which includes a IV bag 1604 having a plurality of entry ports 1603 and an exit port 1605, wherein a plurality of containers 1602 are received and engaged by the plurality of entry ports 1603 to transfer a dose from the plurality of containers 1602 to the IV bag 1604, according to some embodiments of the invention. FIG. 16 shows a ratchet tooth 1608 on the container 1602 that mates and slide the container 1602 from a first position to a second position, which includes a contaminant-free fluid passageway that is hermetically sealed in order to transfer a dose into the IV bag 1604.

After the dose is transferred, the container 1602 slides into a third compartment whereby the container 1602 is able to be removed from the entry port 1603.

In certain embodiments, the third compartment has a resealing element, such that upon entering the third compartment, the distal end of the container 1602 mates with the resealing element. Afterwards, the container 1602 is removed from the entry port 1603.

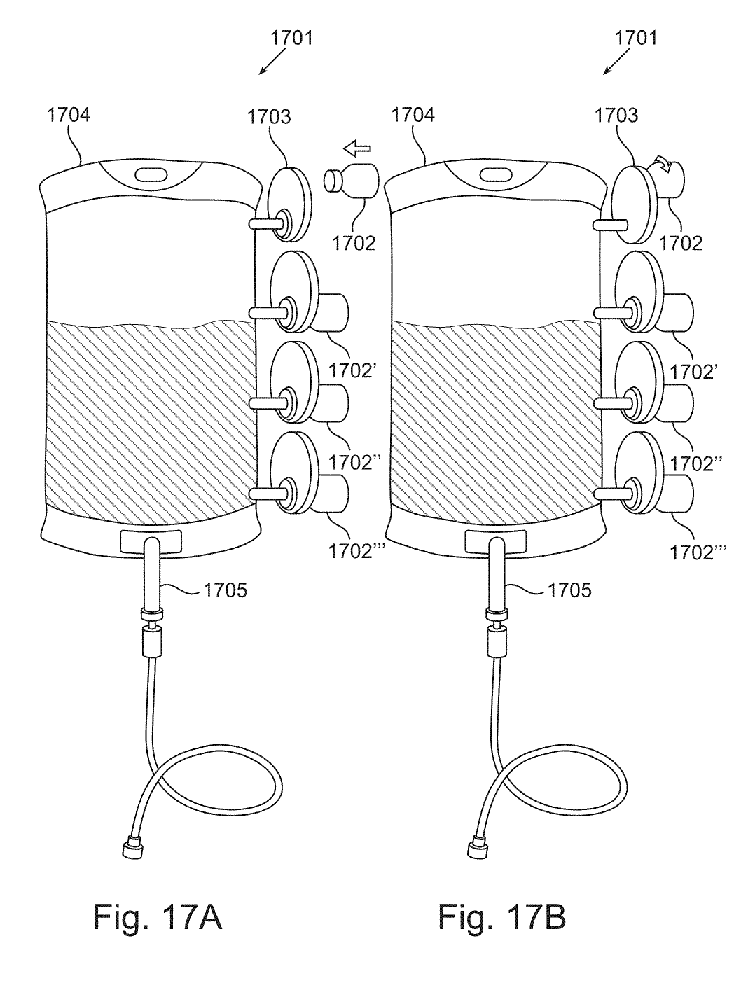

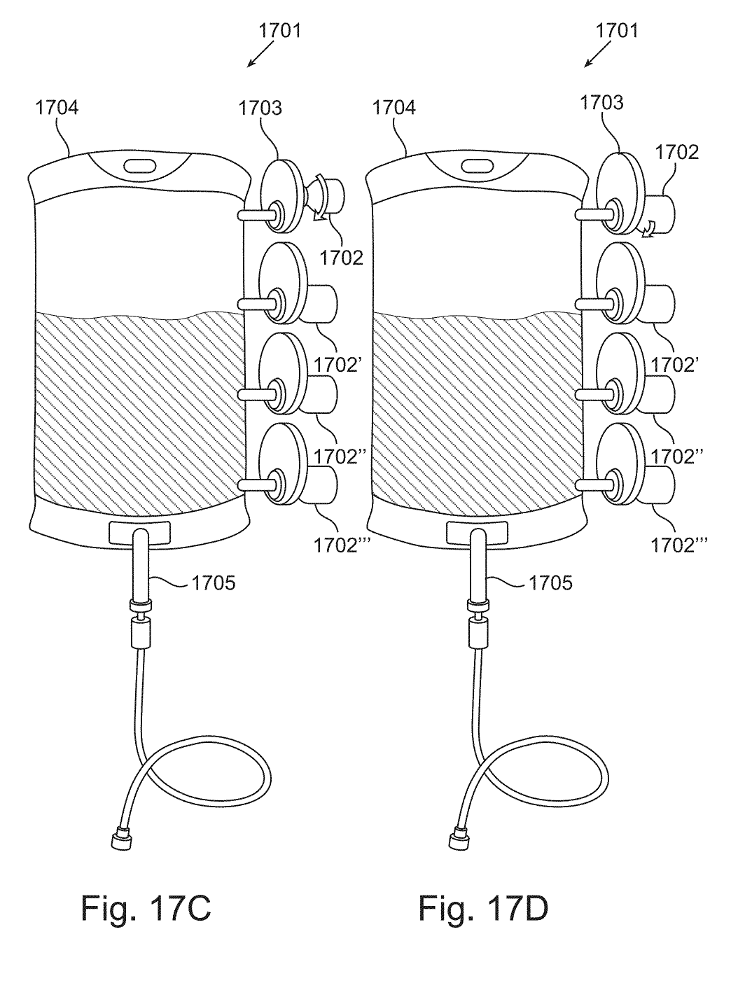

FIGS. 17A-17D are schematic front cut view illustrations presenting a further exemplary system 1701 which includes a IV bag 1704 having a plurality of entry ports 1703 and an exit port 1705, wherein a plurality of containers 1702 are received and engaged by the plurality of entry ports 1703 to transfer a dose from the plurality of containers 1702 to the IV bag 1704, according to some embodiments of the invention. As shown in FIGS. 17A-17D, containers 1702 are vials.

As shown in FIG. 17A, container 1702 and entry port 1703 are separated. In FIG. 17B, container 1702 is engaged with entry port 1703. The container 1702 is able to rotate from a first position in the entry port 1703 to a second compartment in the entry port 1703 as shown in FIGS. 17B-17C. The contaminants are not able to enter the second compartment as a wiping member (not shown) is disposed within entry ports 1703 that removes the contaminants from the surface of container 1702, and the wiping member is within the track between the first and second positions in the entry port 1703. Optionally, wiping member (not shown) is disposed inside the entry ports 1703 and is positioned between a first compartment and a second compartment of entry ports 1703. Entry ports 1703 may be decontamination devices. Optionally, decontamination devices may be abut, be surface mounted or be flush mounted to a wall of IV bag 1704.

FIG. 17D shows container 1702 in the second position whereby the container 1702 is in fluid communication through the entry port 1703 into the IV bag 1704 through which is a contaminant-free fluid passageway.

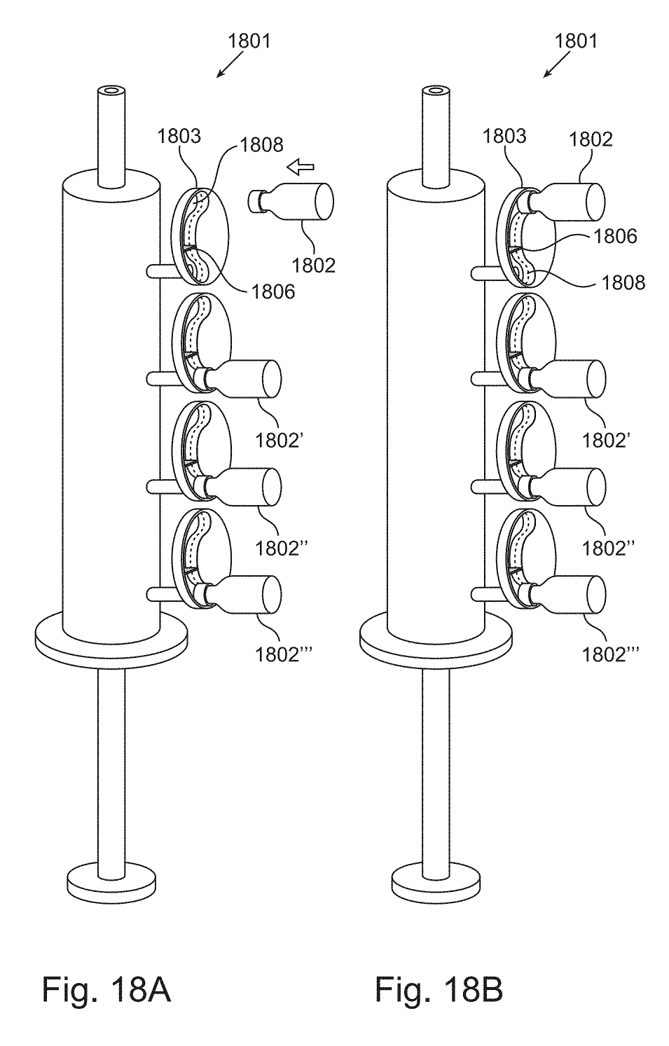

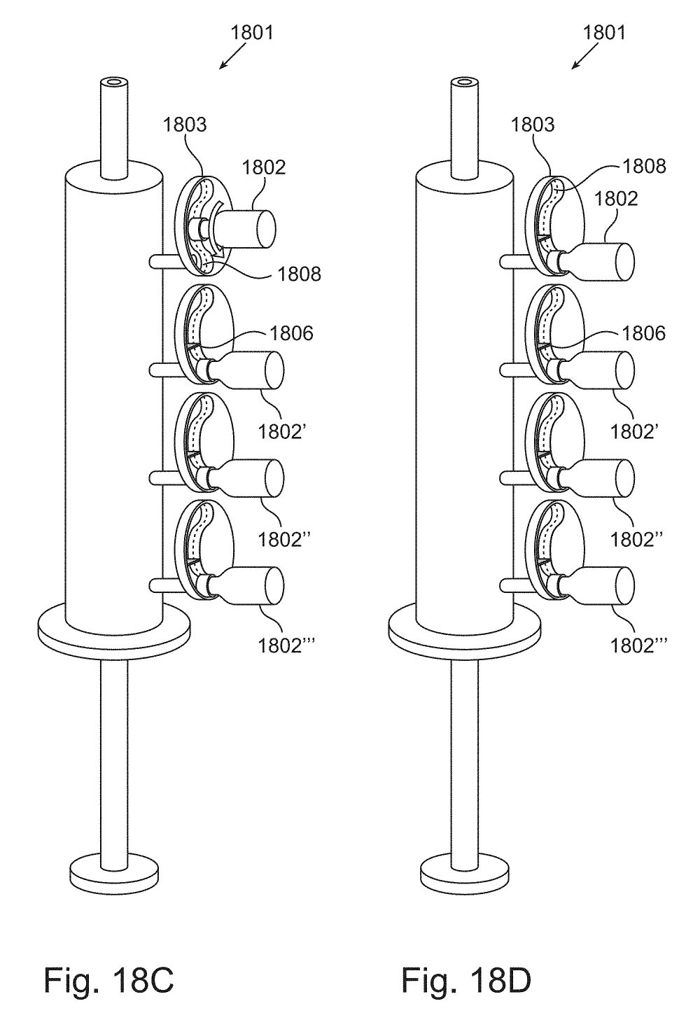

FIGS. 18A-18D are schematic front cut view illustrations presenting a further exemplary system which includes a syringe 1801 having a plurality of entry ports 1803, wherein a plurality of containers 1802 are received and engaged by the plurality of entry ports 1803 to transfer a dose from the plurality of containers 1802 to the syringe 1801, according to some embodiments of the invention. Optionally, the exit port (tip of syringe/syringe tip) may have and/or be attached to a decontamination device. The exit port (tip of syringe/syringe tip) may be integrally attached and/or form a unitary structure with a decontamination device.

As shown in FIG. 18A, container 1802 and entry port 1803 are separated. In FIG. 18B, container 1802 is engaged with entry port 1803. The container 1802 is able to rotate from a first position in the entry port 1803 to a second position in the entry port 1803 as shown in FIGS. 18B-18C. The contaminants are not able to enter the second position as a wiping member 1806 is provided that removes the contaminants from the surface of container 1802, and the wiping member 1806 is within the track 1808 between the first and second positions in the entry port 1803.

FIG. 18D shows container 1802 in the second position whereby the container 1802 is in fluid communication through the entry port 1803 through which a contaminant-free fluid passageway is established into the syringe 1801.

In one or more embodiments, the plurality of entry ports 1803 do not have (are not attached) to decontamination devices. In one or more embodiments, entry ports 1803 are regular entry ports. In one or more embodiments, the syringe has a plurality of entry ports. In one or more embodiments, the syringe has a plurality of entry ports and a plurality of engagement mechanisms configured to engage a plurality of containers 1802.

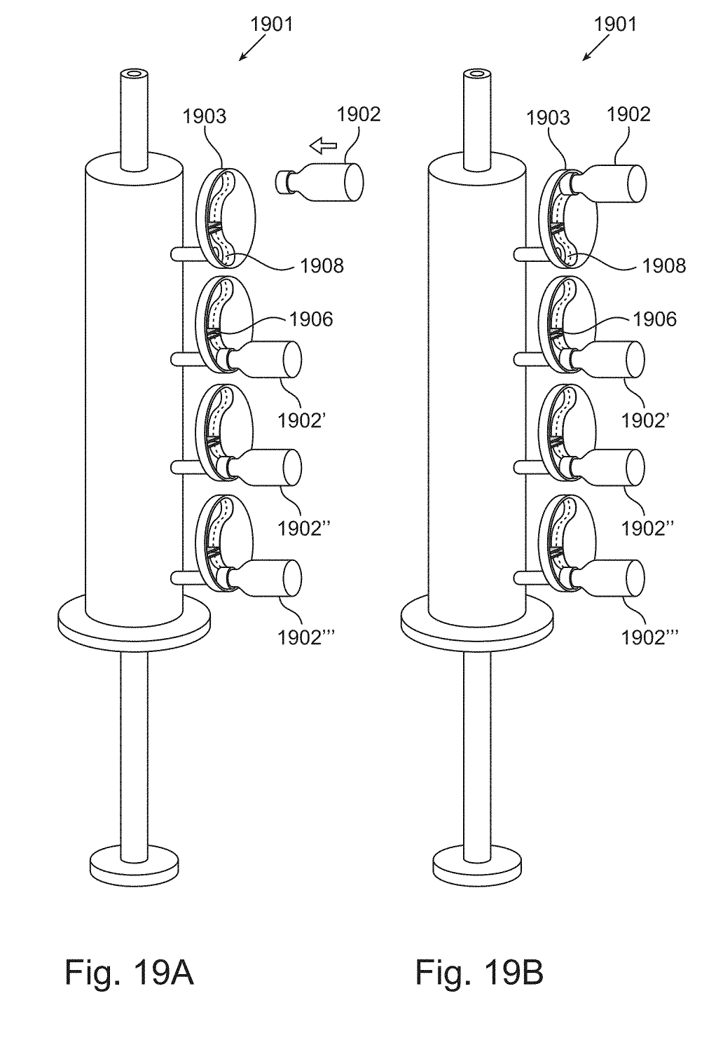

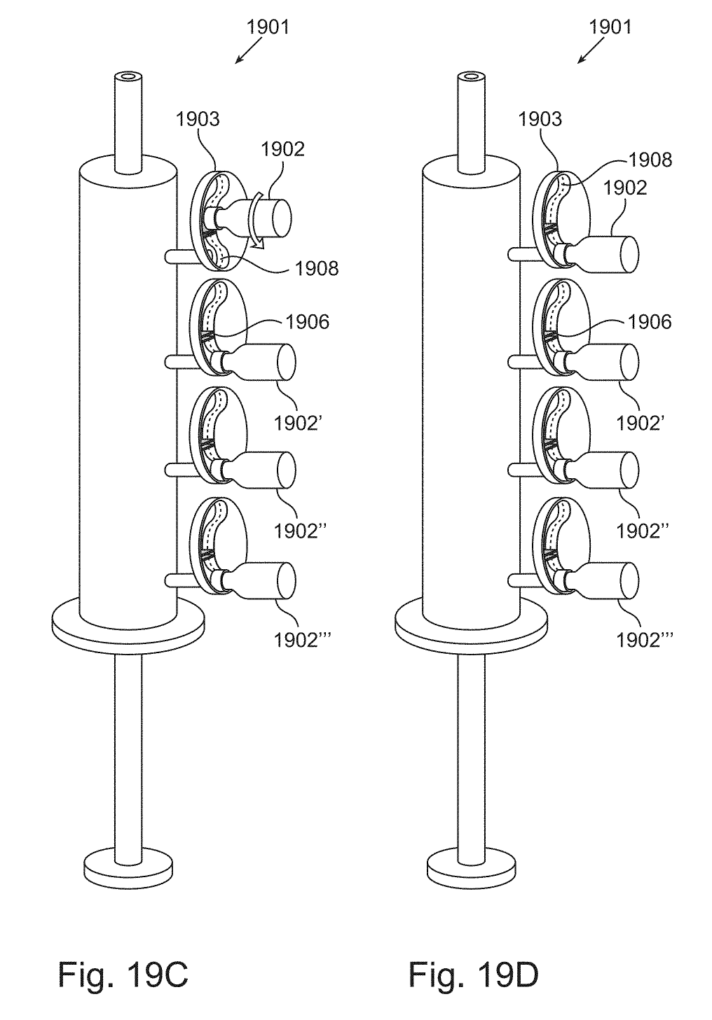

FIGS. 19A-19D are schematic front cut view illustrations presenting a further exemplary system which includes a syringe 1901 having a plurality of entry ports 1903 and, wherein a plurality of containers 1902 are received and engaged by the plurality of entry ports 1903 to transfer a dose from the plurality of containers 1902 to the syringe 1901, according to some embodiments of the invention.

As shown in FIG. 19A, container 1902 and entry port 1903 are separated. In FIG. 19B, container 1902 is engaged with entry port 1903. The container 1902 is able to rotate from a first position in the entry port 1903 to a second position in the entry port 1903 as shown in FIGS. 19B-19C. The contaminants are not able to enter the second position as a double wiping member 1906 is provided that removes the contaminants from the surface of container 1902, and the double wiping member 1906 is within the track 1908 between the first and second positions in the entry port 1903.

FIG. 19D shows container 1902 in the second position whereby the container 1902 is in fluid communication through the entry port 1903 into the syringe 1901 through which is a contaminant-free fluid passageway.

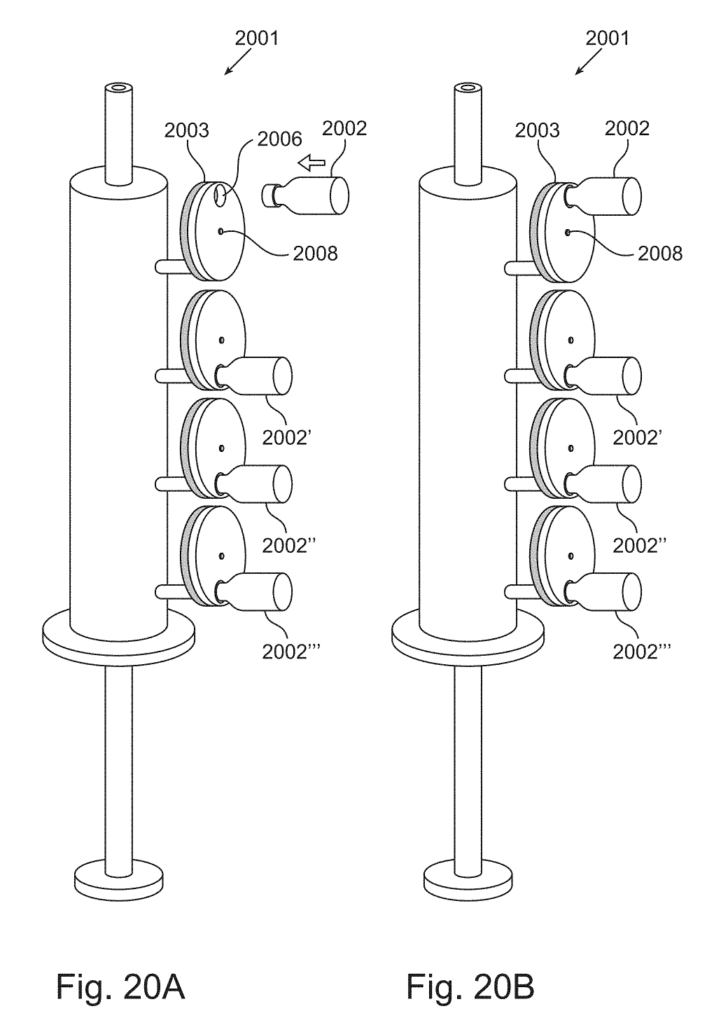

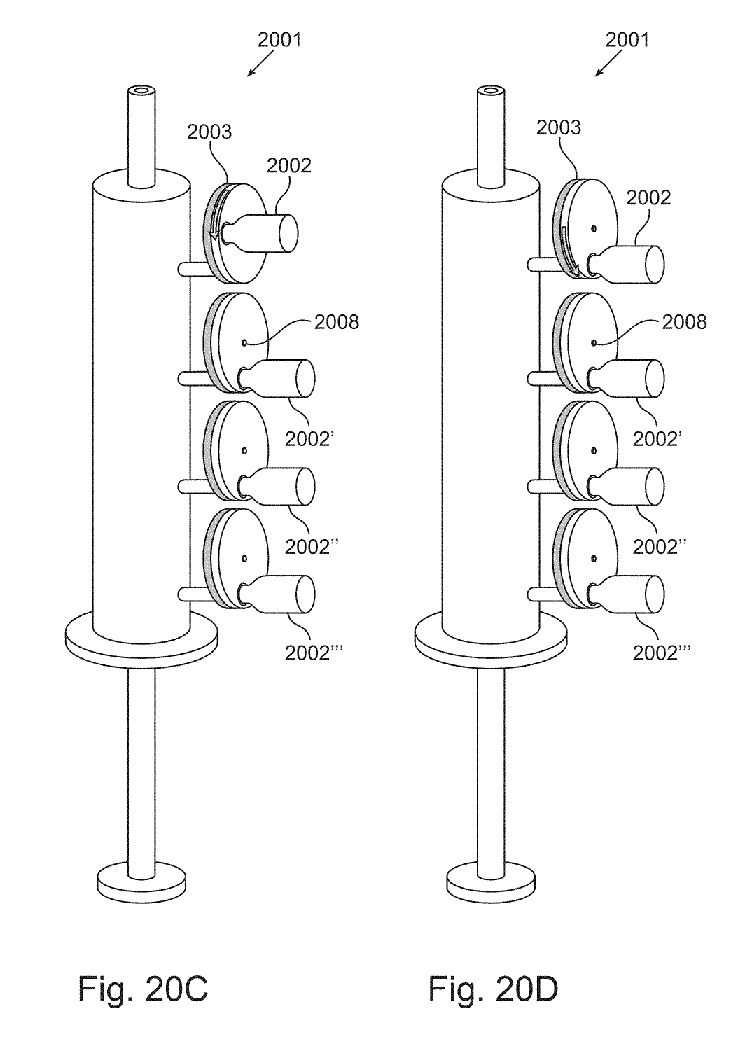

FIGS. 20A-20D are schematic perspective view illustrations presenting a further exemplary system which includes a syringe 2001 having a plurality of entry ports 2003 and an exit port (syringe tip), wherein a plurality of containers 2002 are received and engaged by the plurality of entry ports 2003 to transfer a dose from the plurality of containers 2002 to the syringe 2001, according to some embodiments of the invention.

As shown in FIG. 20, container 2002 and entry port 2003 are separated. In FIG. 20B, container 2002 is engaged with entry port 2003 via entry port opening 2006. Entry port opening 2006 may be covered or sealed prior to attachment of container 2002 to entry port 2003. The cover or seal may be a frangible seal or a removeable cover. The container 2002 is able to rotate from a first position in the entry port 2003 to a second position in the entry port 2003 as shown in FIGS. 20B-20C. As shown, entry port 2003 is composed of two disks that rotate with respect to one another from a first position to a second position. The two discs may rotate with respect to each other via a hinge mechanism 2008 that connects the two discs and provides for the rotation of at least one of the two discs in relation to the other disc. Optionally, the two discs may rotate with respect to each other via a circumferential rail mechanism (not shown) on at least one of the two discs which engages the other disc. The two discs form an airtight and/or hermetic engagement. The contaminants are not able to enter the second position as there is a wiping member disposed between the two discs removes the contaminants from the surface of container 2002, and the wiping member 2006 is within a track 2008 between the first and second positions in the entry port 2003.

FIG. 20D shows container 2002 in the second position whereby the container 2002 is in fluid communication through the entry port 2003 into the syringe 2001 through which is a contaminant-free fluid passageway.

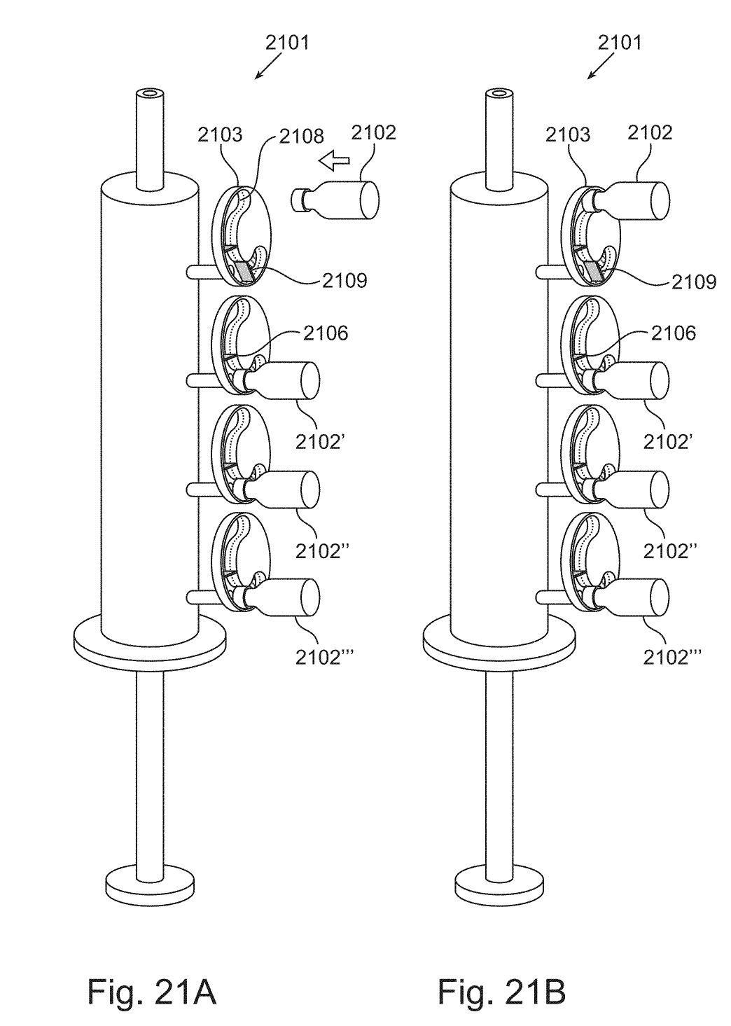

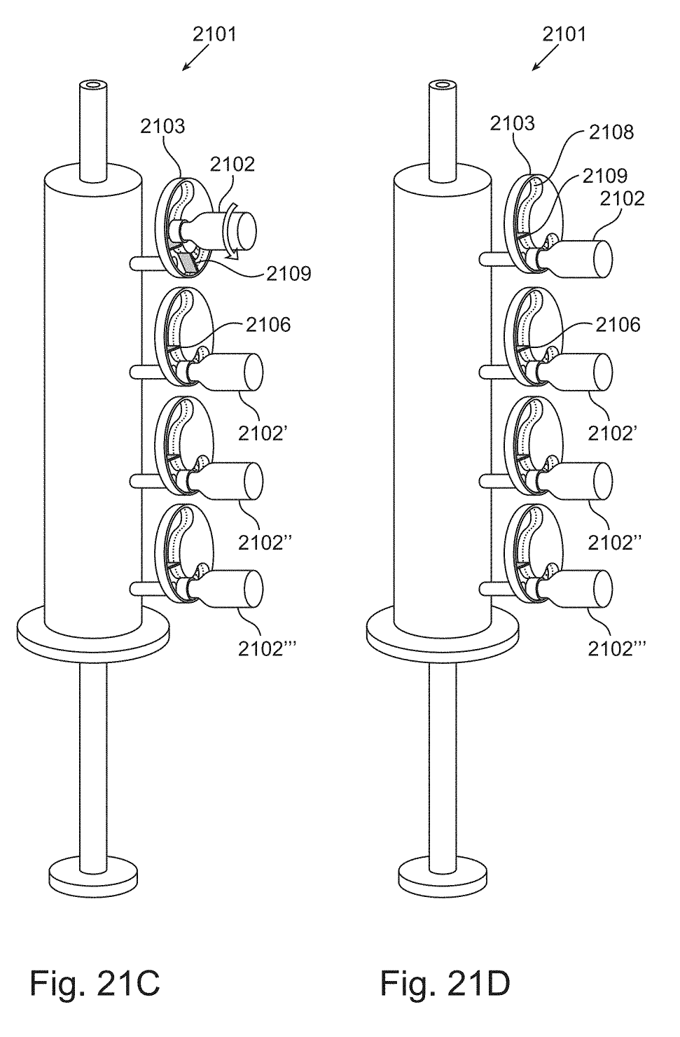

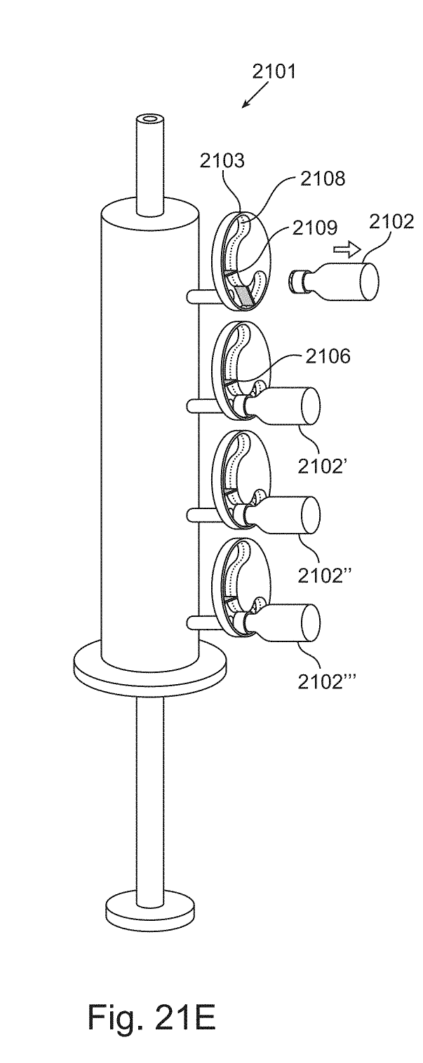

FIGS. 21A-21E are schematic front cut view illustrations presenting a further exemplary system which includes a syringe 2101 having a plurality of entry ports 2103, wherein a plurality of containers 2102 are received and engaged by the plurality of entry ports 2103 to transfer a dose from the plurality of containers 2102 to the syringe 2101, according to some embodiments of the invention.

As shown in FIG. 21, container 2102 and entry port 2103 are separated. In FIG. 21B, container 2102 is engaged with entry port 2103. The container 2102 is able to rotate from a first position in the entry port 2103 to a second position in the entry port 2103 as shown in FIGS. 21B-21C. The contaminants are not able to enter the second position as there is a wiping member 2106 that removes the contaminants from the surface of container 2102, and the wiping member 2106 is within a track 2108 between the first and second positions in the entry port 2103.

FIG. 21D shows container 2102 in the second position whereby the container 2102 is in fluid communication through the entry port 2103 into the syringe 2101 through which is a contaminant-free fluid passageway. FIG. 21E shows the container (or vial) 2102 removed from the track 2108 by pulling the container away from the syringe 2101.

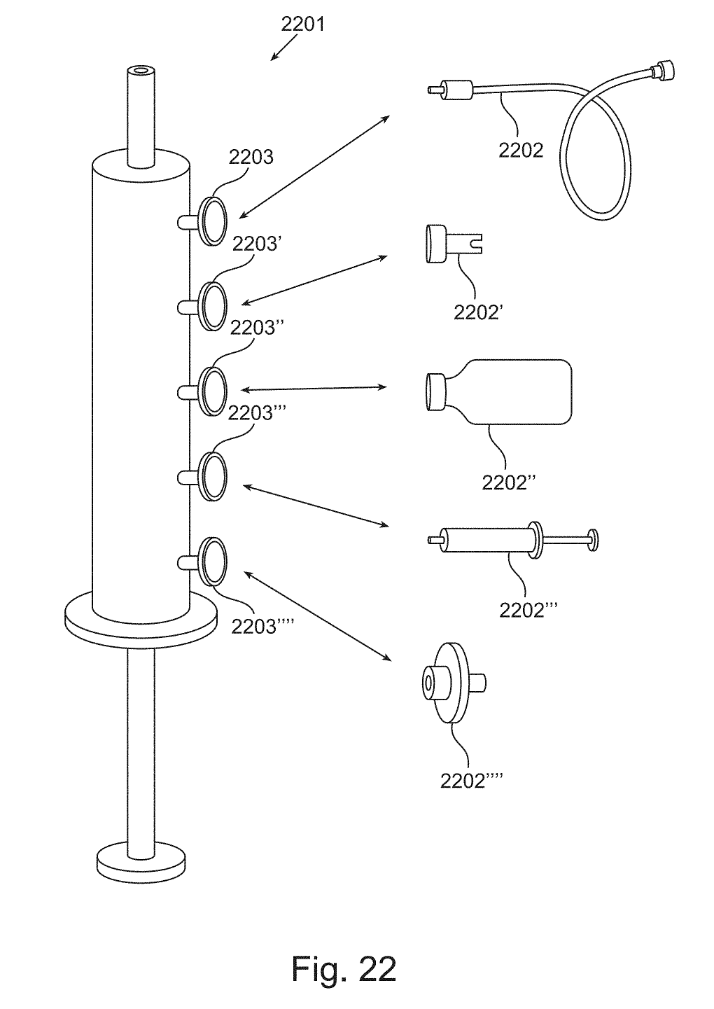

FIG. 22 is a schematic front cut view illustration presenting a further exemplary system which includes a syringe 2201 having a plurality of entry ports 2203, showing various objects 2202, 2202′, 2202″, 2202′″ and 2202″″ able to be received by the plurality of entry ports 2203, according to some embodiments of the invention. As shown the plurality of objects include an IV line, filter, bung, bottle, syringe, connector and other objects that are configured to interface or connect with entry ports 2203.

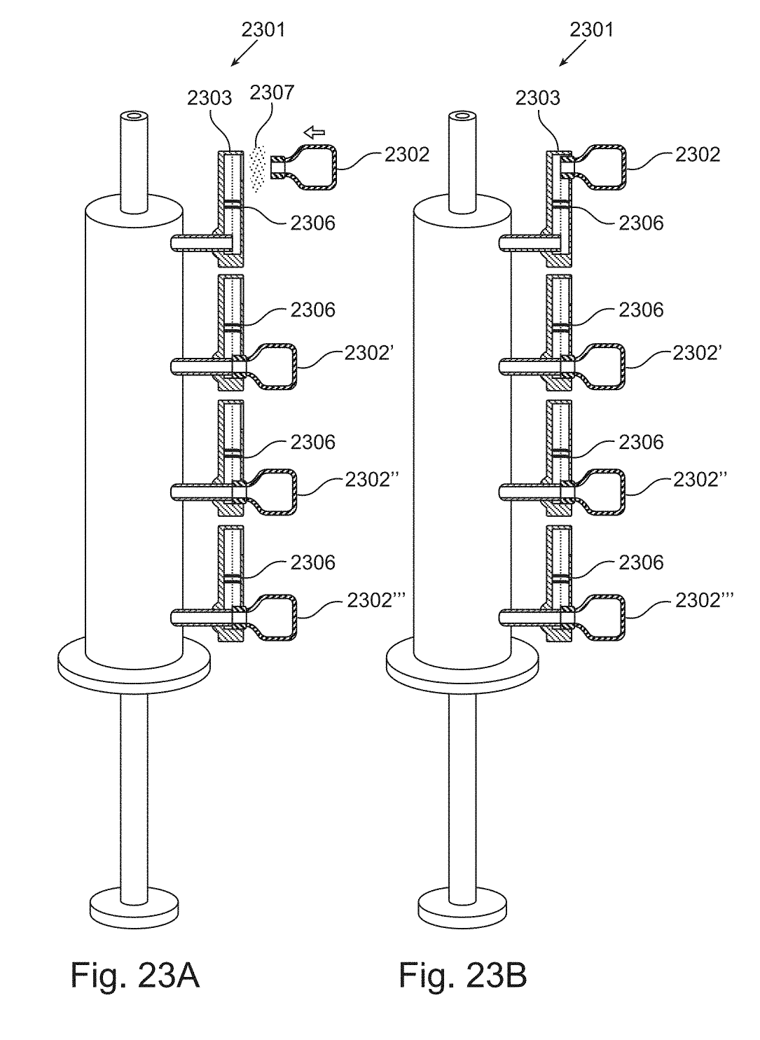

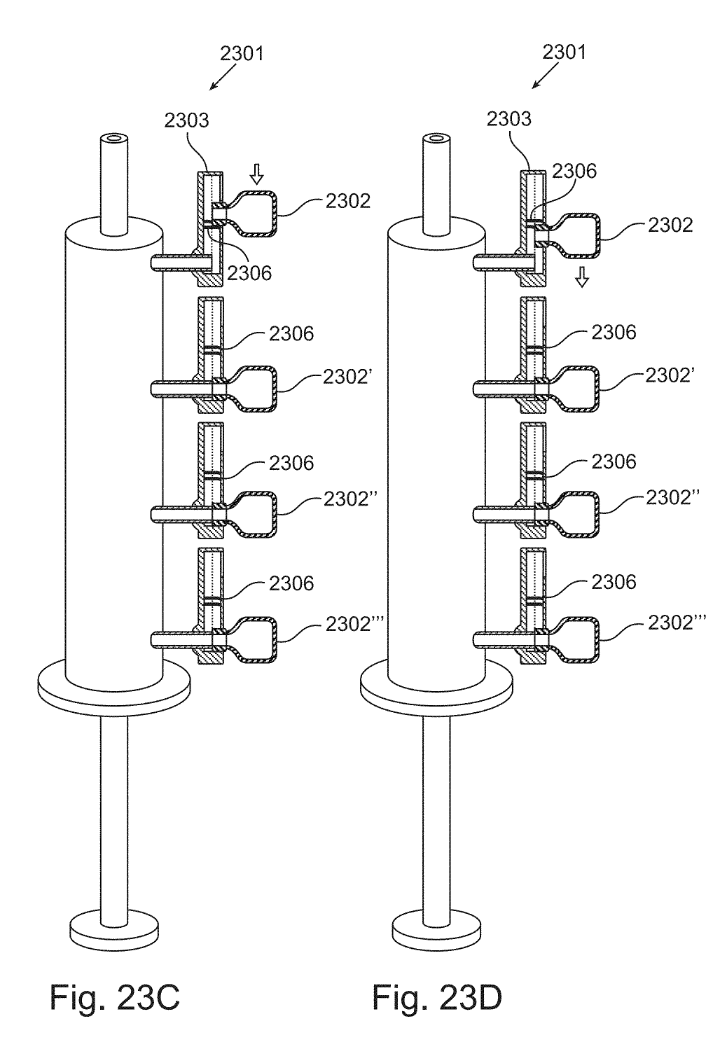

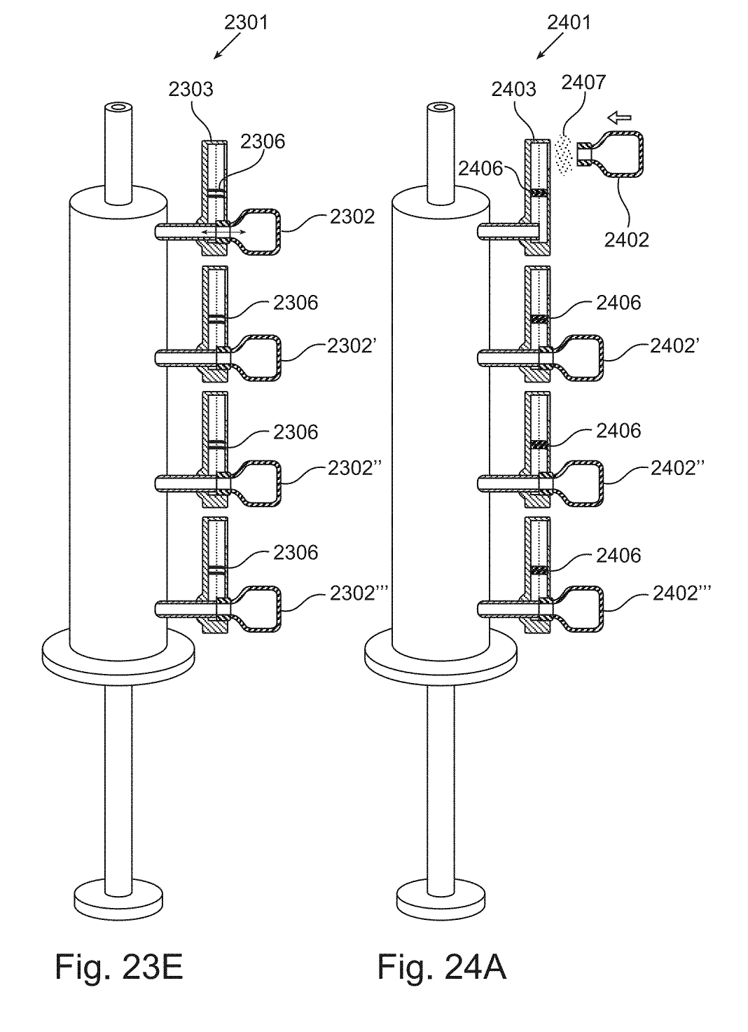

FIGS. 23A-23E are schematic front cut view illustrations presenting a further exemplary system which includes a syringe 2301 having a plurality of entry ports 2303, wherein a plurality of containers 2302 are received and engaged by the plurality of entry ports 2303 to transfer a dose from the plurality of containers 2302 to the syringe 2301, according to some embodiments of the invention.

As shown in FIG. 23A, container 2302 and entry port 2303 are separated and contaminates 2307 are shown on the distal tip of container 2302. In FIG. 23B, container 2302 is engaged with entry port 2303. The container 2302 is able to slide from a first compartment in the entry port 2303 to a second compartment in the entry port 2303 as shown in FIGS. 23C-23D. The contaminants 2307 are not able to enter the second compartment as a double wiping member 2306 is provided that removes the contaminants from the surface of container 2302. Double wiping member 2306 is disposed within entry port 2303 of syringe 2301.

FIG. 23E shows the transfer of dose from the container 2302 through the entry port 2303 into the syringe 2301 through which is a contaminant-free fluid passageway in the second compartment.

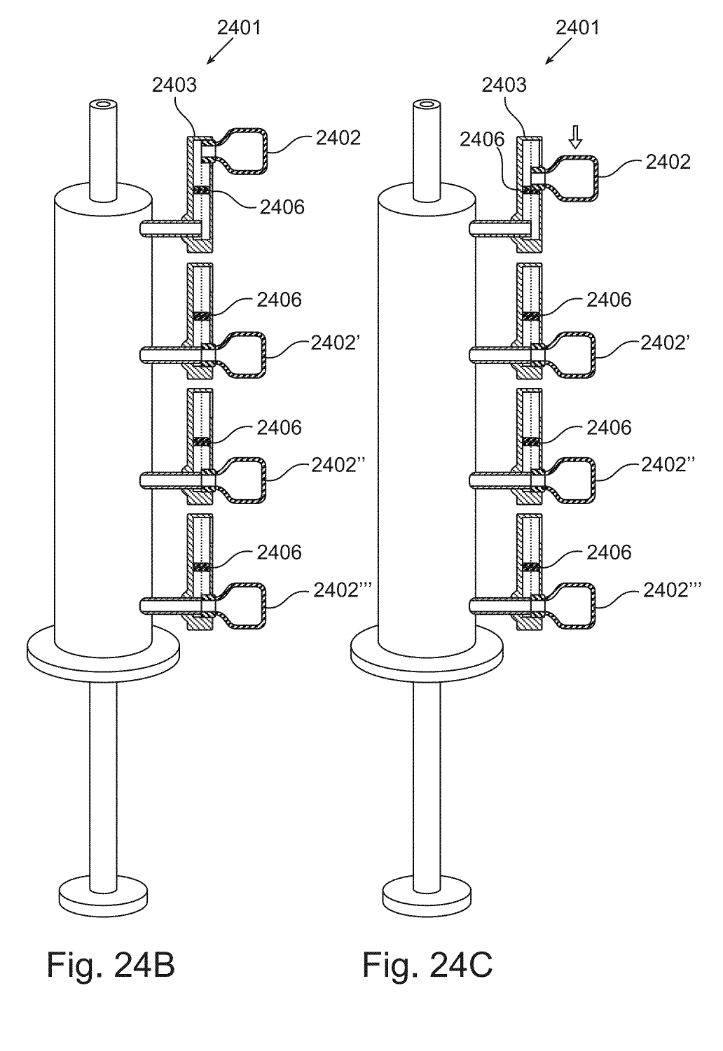

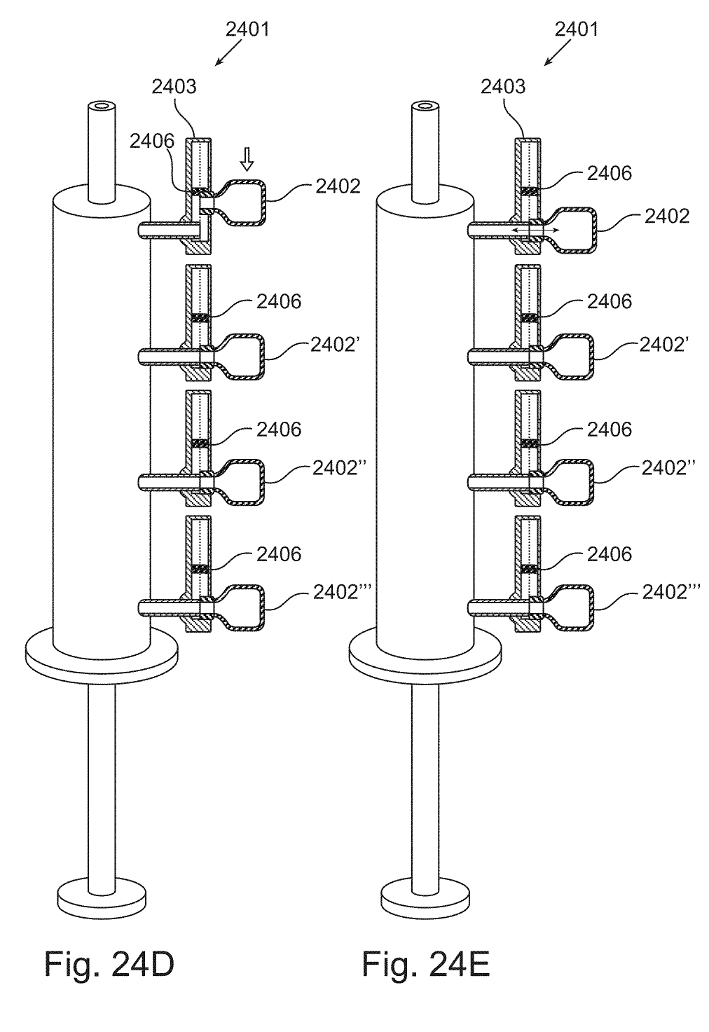

FIGS. 24A-24E are schematic front cut view illustrations presenting a further exemplary system which includes a syringe 2401 having a plurality of entry ports 2403, wherein a plurality of containers 2402 are received and engaged by the plurality of entry ports 2403 to transfer a dose from the plurality of containers 2402 to the syringe 2401, according to some embodiments of the invention.

As shown in FIG. 24A, container 2402 and entry port 2403 are separated and contaminates 2407 are shown on the distal tip of container 2402. In FIG. 24B, container 2402 is engaged with entry port 2403. The container 2402 is able to slide from a first compartment in the entry port 2403 to a second compartment in the entry port 2403 as shown in FIGS. 24C-24D. The contaminants 2407 are not able to enter the second compartment as a wiping member 2406 is provided that removes the contaminants from the surface of container 2402. Wiping member 2406 is a double wiping member, wherein the wipers are adjacent to each other and disposed within entry port 2403 of syringe 2401.

FIG. 24E shows the transfer of dose from the container 2402 through the entry port 2403 into the syringe 2401 through which is a contaminant-free fluid passageway in the second compartment.

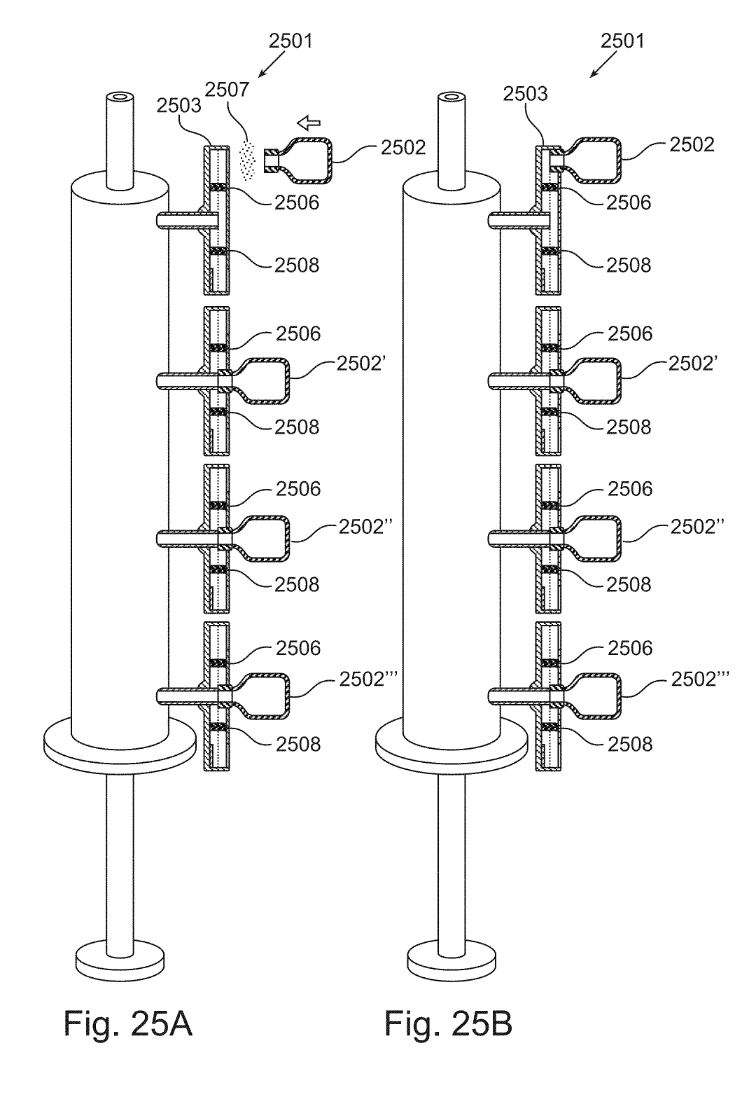

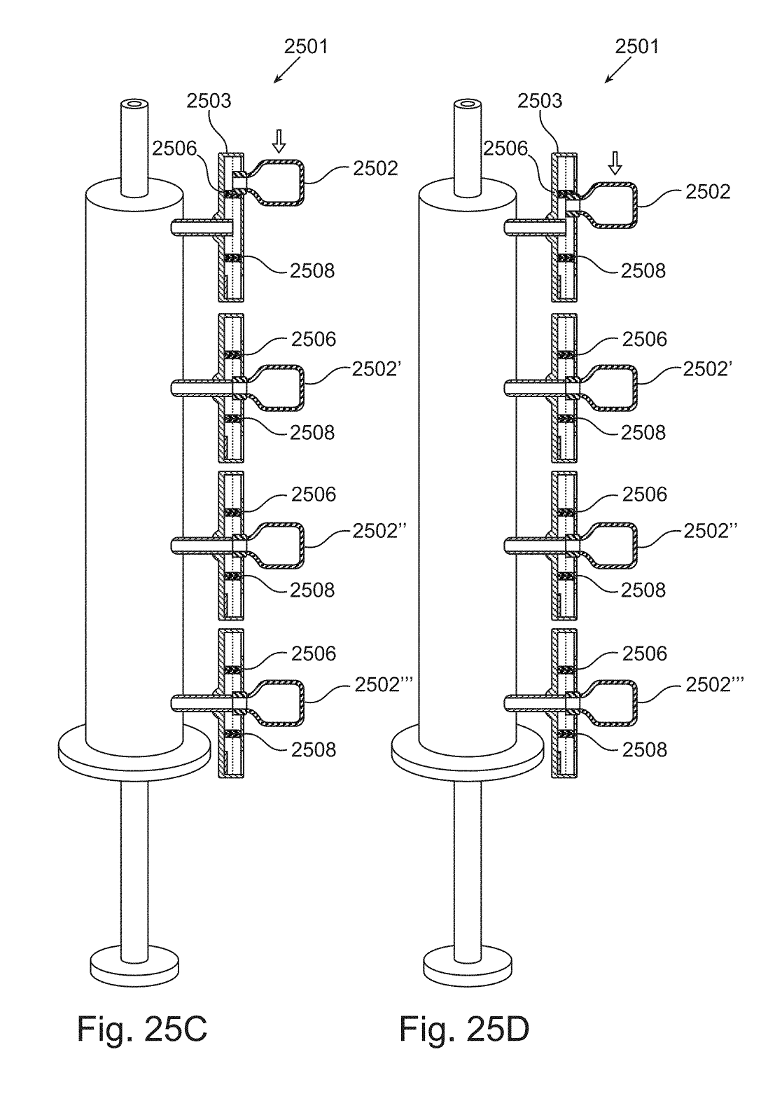

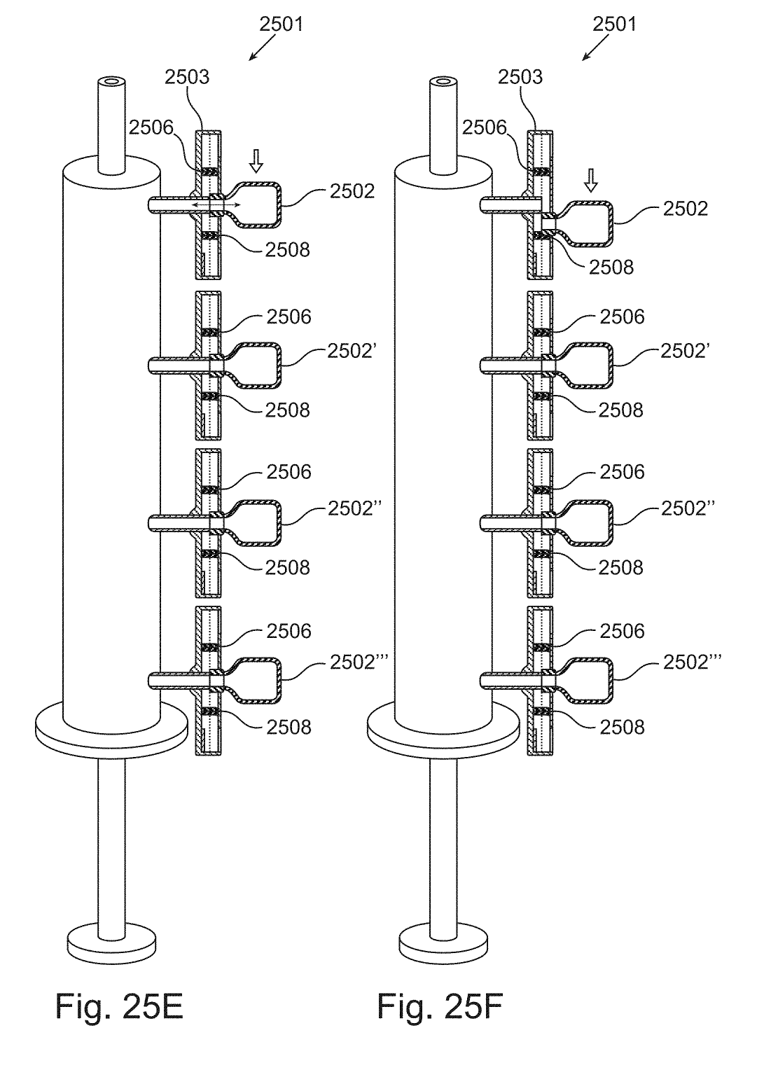

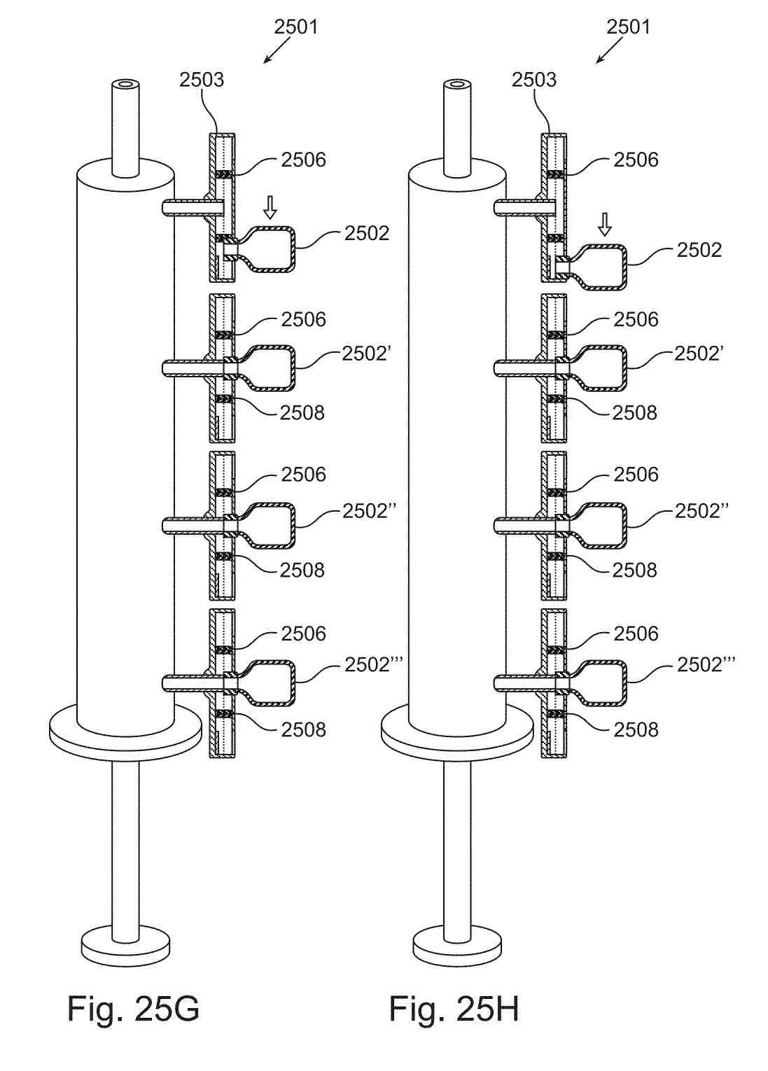

FIGS. 25A-25I are schematic front cut view illustrations presenting a further exemplary system which includes a syringe 2501 having a plurality of entry ports 2503, wherein a plurality of containers 2502 are received and engaged by the plurality of entry ports 2503 to transfer a dose from the plurality of containers 2502 to the syringe 2501, according to some embodiments of the invention.

As shown in FIG. 25A, container 2502 and entry port 2503 are separated and contaminates 2507 are shown on the distal end of container 2502. In FIG. 25B, container 2502 is engaged with entry port 2503. The container 2502 is able to slide from a first compartment in the entry port 2503 to a second compartment in the entry port 2503 as shown in FIGS. 25C-25D. The contaminants 2507 are not able to enter the second compartment as a wiping member 2506 is provided that removes the contaminants from the surface of container 2502.

FIG. 25E shows the transfer of dose from the container 2502 through the entry port 2503 into the syringe 2501 through which is a contaminant-free fluid passageway in the second compartment.

After the dose is transferred, FIGS. 25F-25H shows the container 2502 sliding to a third compartment. A wiping member 2508 is used to decontaminate the distal tip of container 2502 prior to passing into the third compartment. FIGS. 25A-25I show a first double wiping member 2506 consisting of two wiping members adjacent to each other and a second double wiping member 2508 consisting of two wiping members adjacent to each other.

In certain embodiments, the third compartment has a resealing element, such that upon entering the third compartment, the distal end of the container 2502 mates with the resealing element. Afterwards, the container 2502 is removed from the entry port 2503 as shown in FIG. 25I. The resealing element may be selected from a group consisting of a thread, a luer, a luer-lock, a luer-slip, a snap-on mechanism, a snap-on mechanism, a rail mechanism and combinations thereof. The resealing element is disposed in entry port 2503. Optionally, the resealing element may be disposed in a second, in a third, in a fourth or in a fifth compartment of entry port 2503 (not shown).

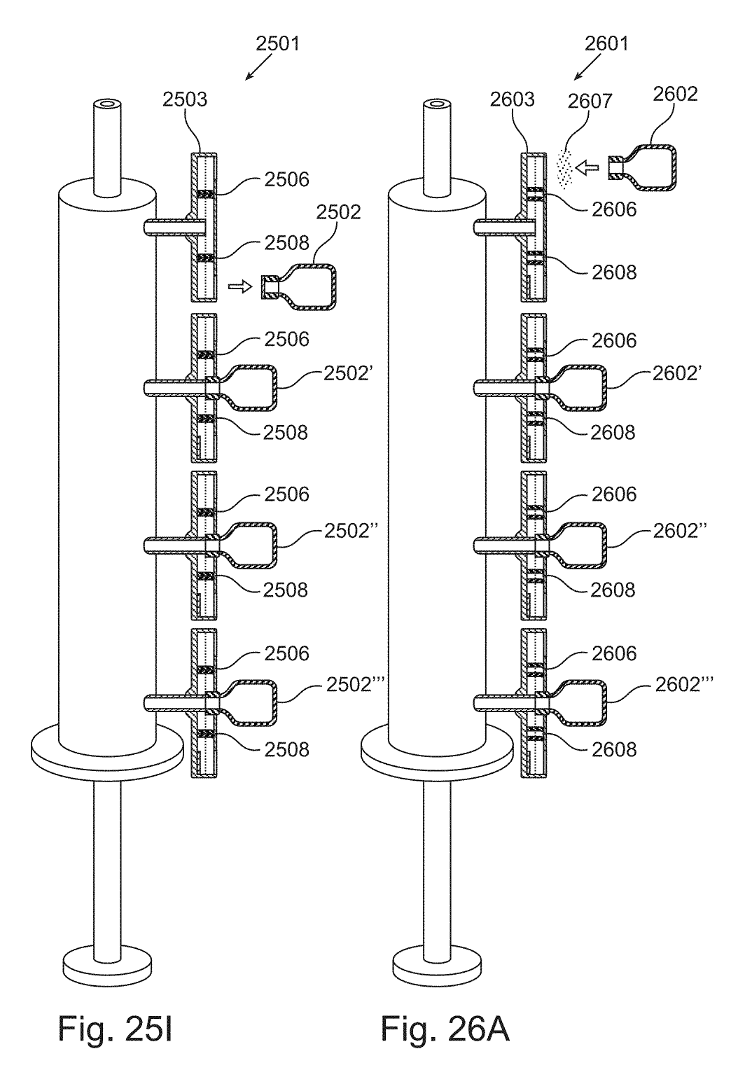

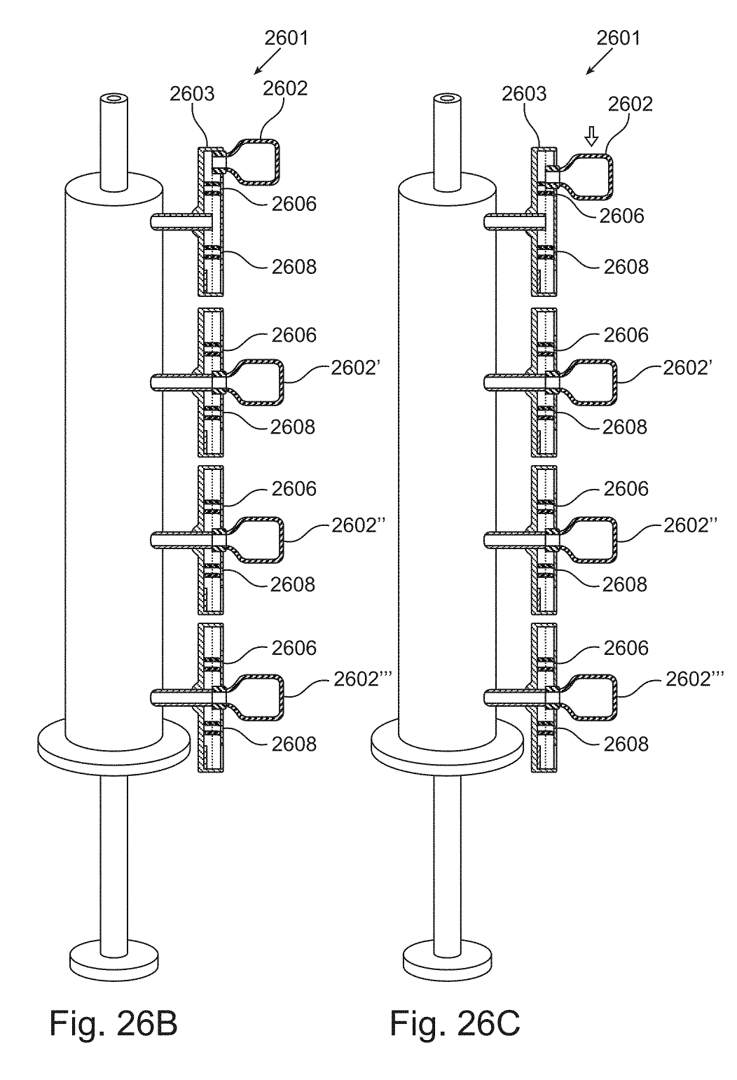

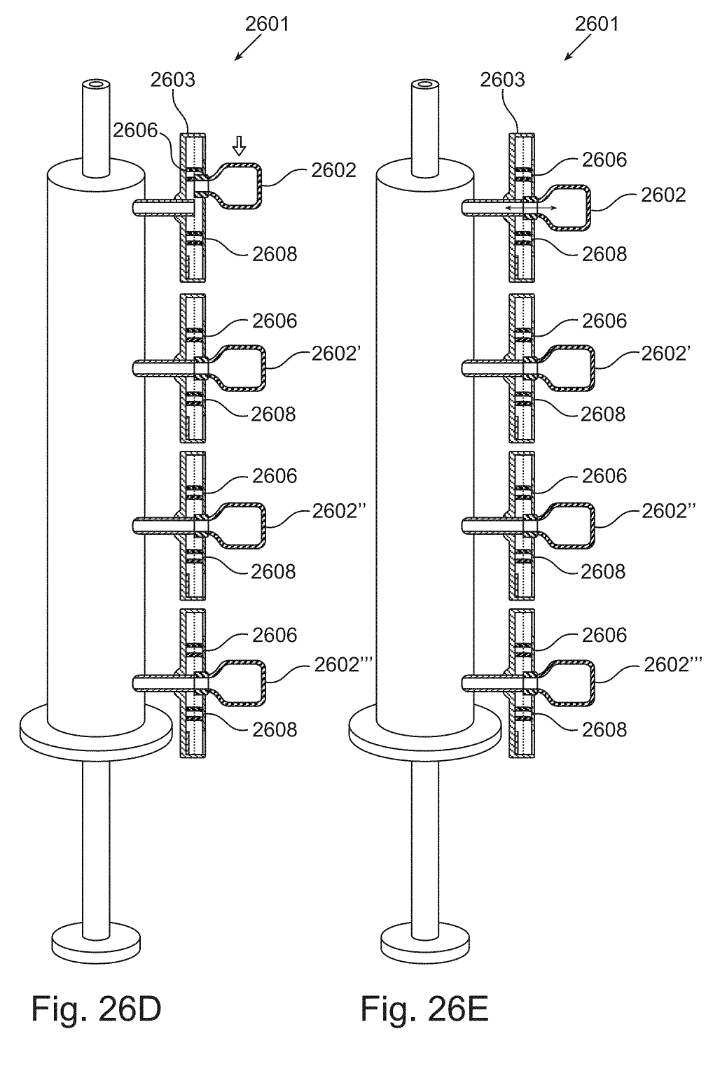

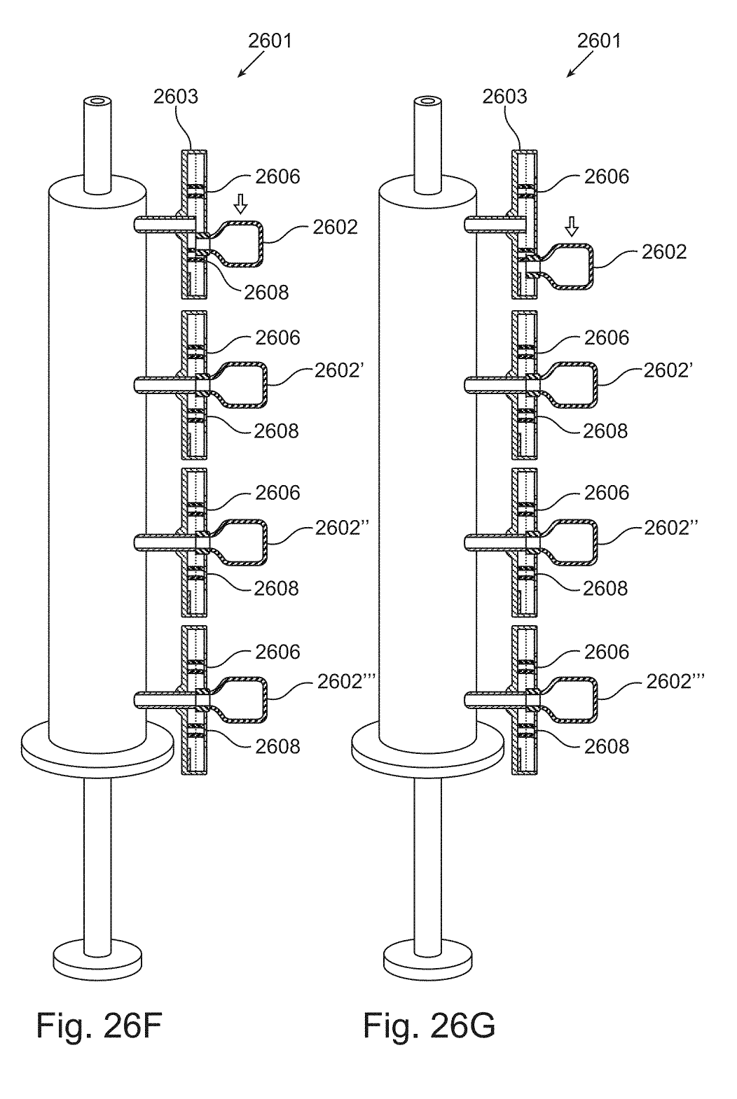

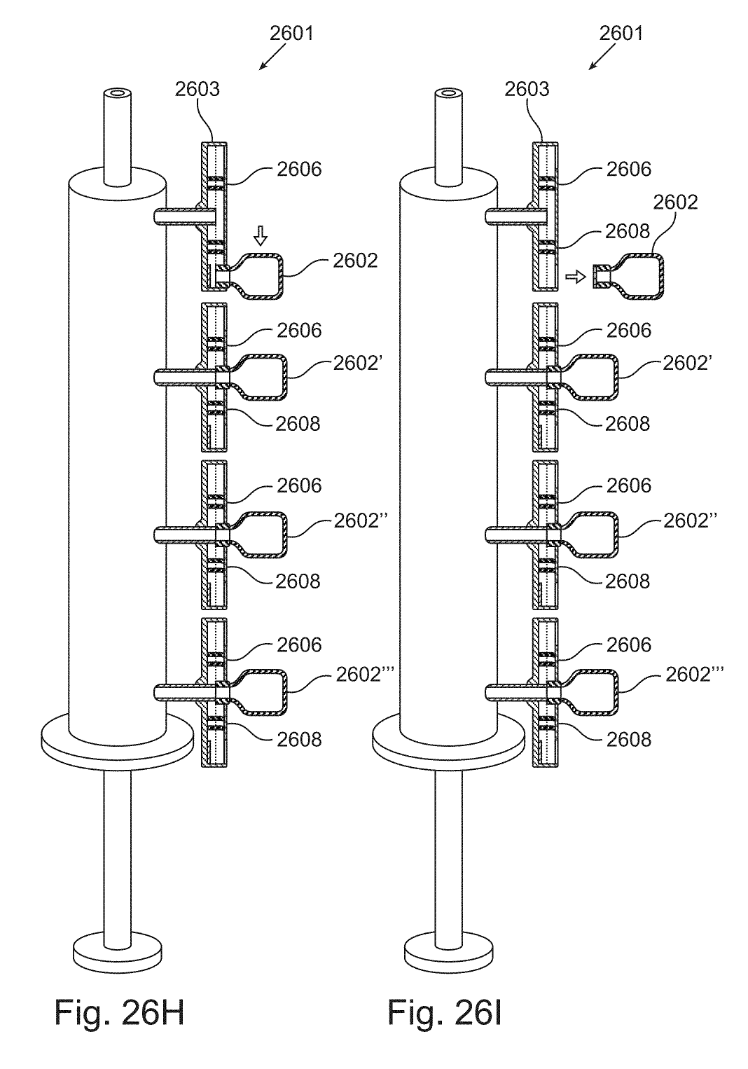

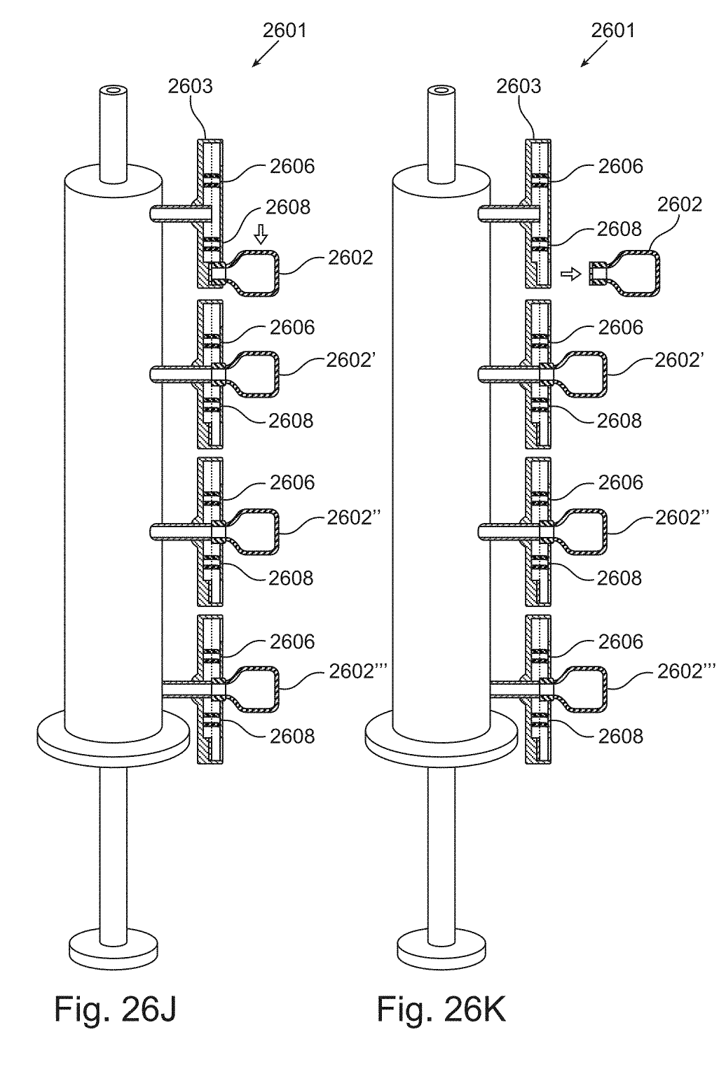

FIGS. 26A-26K are schematic front cut view illustrations presenting a further exemplary system which includes a syringe 2601 having a plurality of entry ports 2603, wherein a plurality of containers 2602 are received and engaged by the plurality of entry ports 2603 to transfer a dose from the plurality of containers 2602 to the syringe 2601, according to some embodiments of the invention.

As shown in FIG. 26A, container 2602 and entry port 2603 are separated and contaminates 2607 are shown on the distal tip of container 2602. In FIG. 26B, container 2602 is engaged with entry port 2603. The container 2602 is able to slide from a first compartment in the entry port 2603 to a second compartment in the entry port 2603 as shown in FIGS. 26C-26D. The contaminants 2607 are not able to enter the second compartment as a double wiping member 2606 is provided that removes the contaminants from the surface of container 2602.

FIG. 26E shows the transfer of dose from the container 2602 through the entry port 2603 into the syringe 2601 through which is a contaminant-free fluid passageway in the second compartment.

After the dose is transferred, FIGS. 26F-26I shows the container 2602 sliding to a third compartment. A double wiping member 2608 is used to decontaminate the distal tip of container 2602 prior to passing into the third compartment.

In certain embodiments, the third compartment has a resealing element, such that upon entering the third compartment, the distal end of the container 2602 mates with the resealing element. Afterwards, the container 2602 is removed from the entry port 2603 as shown in FIGS. 2J-26K. The first double wiping member 2606 and the second double wiping member 2608 are characterized in the double wipers are spaced apart from each other.

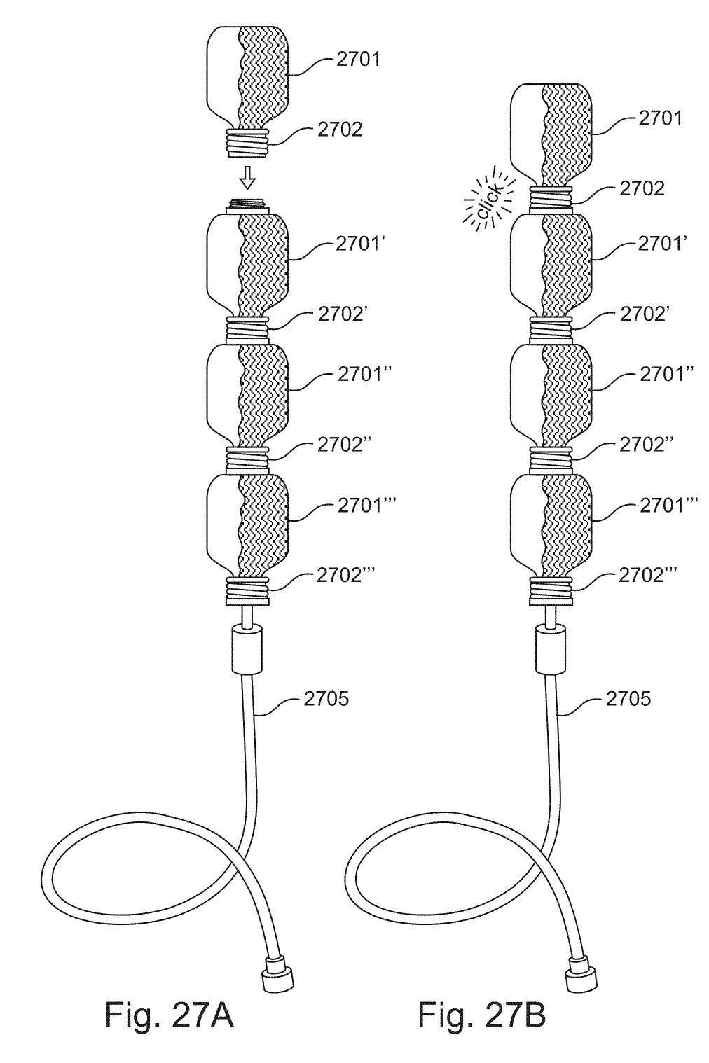

FIGS. 27A-27B are schematic front cut view illustrations presenting a further exemplary system which includes a modular dosing system of a plurality of containers 2701, whereby the containers 2701 are configured to be connected to one another, according to some embodiments of the invention.

As shown in FIG. 27A-27B, a container 2701 is shown having thread 2702. A second container 2701′ is shown having an upper thread and a bottom thread 2702′. The thread 2702 of the first container 2701 mates with the upper thread of the second container 2701′ in order to place the container 2701 and 2701′ in fluid communication with one another. Additional containers 2701″ and 2701′ are mated together in a similar series configuration terminating in IV line 2705.

FIG. 27B shows that when container 2701 is mated with container 2701′, a “click” or any other sound is provided to know when the containers are engaged and/or hermetically sealed with one another. When the plurality of containers 2701 are in fluidic communication with one another a force of gravity pulls the contents of the plurality of containers down into the infusion line. The containers 2701 may house a beneficial substance and/or a medicament in nonstandard amounts, standard amounts and combinations thereof. Contents of the first container 2701 flow down through the second container 2701′, flow down through the third container 2701″ and flow down through the fourth container 2701′″ into an infusion line 2705. In this embodiment, the second and third containers house a beneficial substance as well as provide a fluidic passageway for the flow of contents of container 2701 to flow through container 2701′ and 2701″ into container 2701′″. Optionally, the infusion line 2705 may not be present. Engagement between containers 2701 may be airtight and/or hermetic.

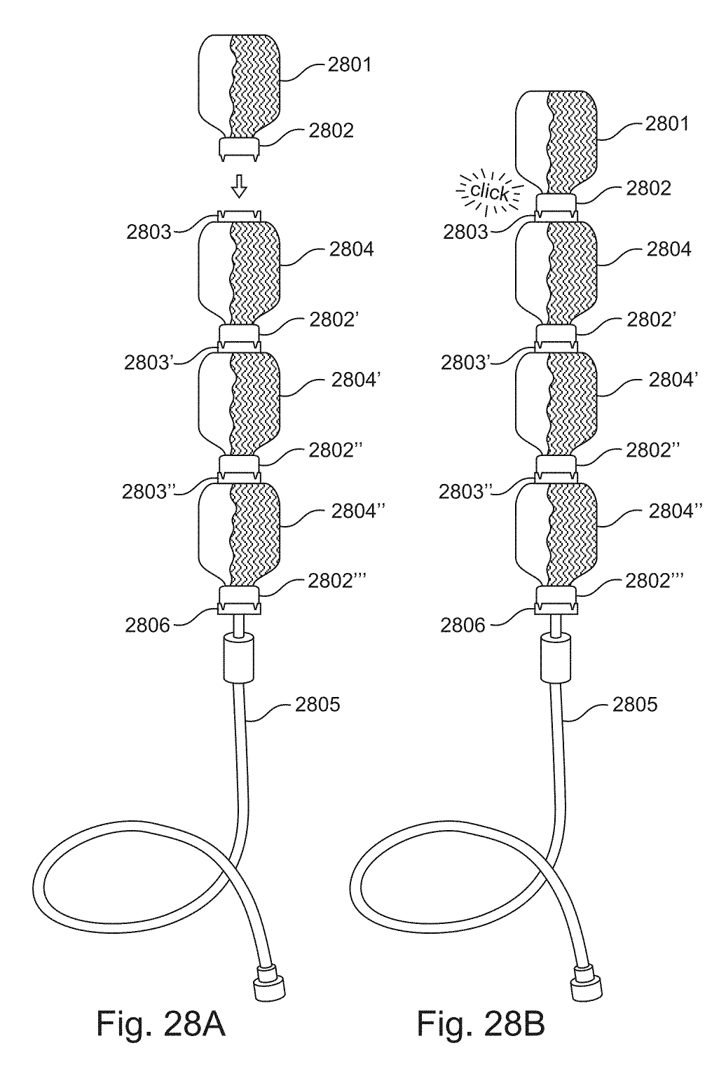

FIGS. 28A-28B are schematic front cut view illustrations presenting a further exemplary system which includes a modular dosing system of a plurality of containers 2801, 2804, 2804′, and 2804″, whereby the containers 2801, 2804, 2804′ and 2804″ are configured to be connected to one another, according to some embodiments of the invention. In certain embodiments, the system of FIG. 28A-28B is a modular system providing for the customization of a final amount of a beneficial substance provided to a patient.

As shown in FIG. 28, a first container 2801 is shown having ratchet teeth 2802. A second container 2804 is shown having an upper ratchet tooth 2803 and a bottom ratchet tooth 2802′. The ratchet teeth 2802 of the first container 2801 mates with the upper ratchet teeth 2803 of the second container 2804 in order to place the container 2801 and 2804 in fluid communication with one another. Additional containers 2804′ and 2804″ are mated together in a similar series configuration terminating in IV line 2805.

FIG. 28B shows that when container 2801 is mated with container 2804, a “click” sound is provided to alert a user when the containers are engaged and/or hermetically sealed with one another. In certain embodiments, other sounds may be produced to alert a user when the containers are engaged.

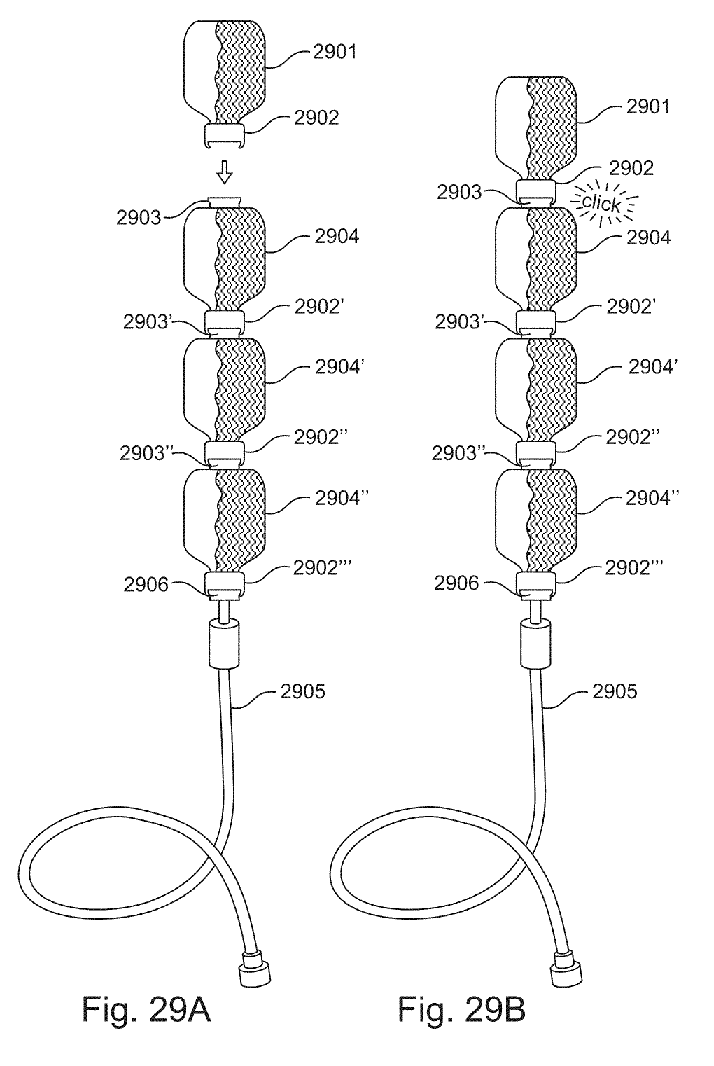

FIGS. 29A-29B are schematic front cut view illustrations presenting a further exemplary system which includes a modular dosing system of a plurality of containers 2901, 2904, 2904′, and 2904″ whereby the containers 2901, 2904, 2904′, and 2904″ are configured to be connected to one another, according to some embodiments of the invention.

As shown in FIG. 29, a container 2901 is shown having ratchet teeth 2902. A second container 2904 is shown having a retention member 2903 and a ratchet tooth 2902′. The ratchet teeth 2902 of the first container 2901 mates with the retention member 2903 of the second container 2904 in order to place the container 2901 and 2904 in fluid communication with one another. Additional containers 2904′ and 2904″ are mated together in a similar series configuration, optionally, terminating in IV line 2905.

FIG. 29B shows that when container 2901 is mated with container 2904, a “click” sound is provided to know when the containers are engaged and/or hermetically sealed with one another.

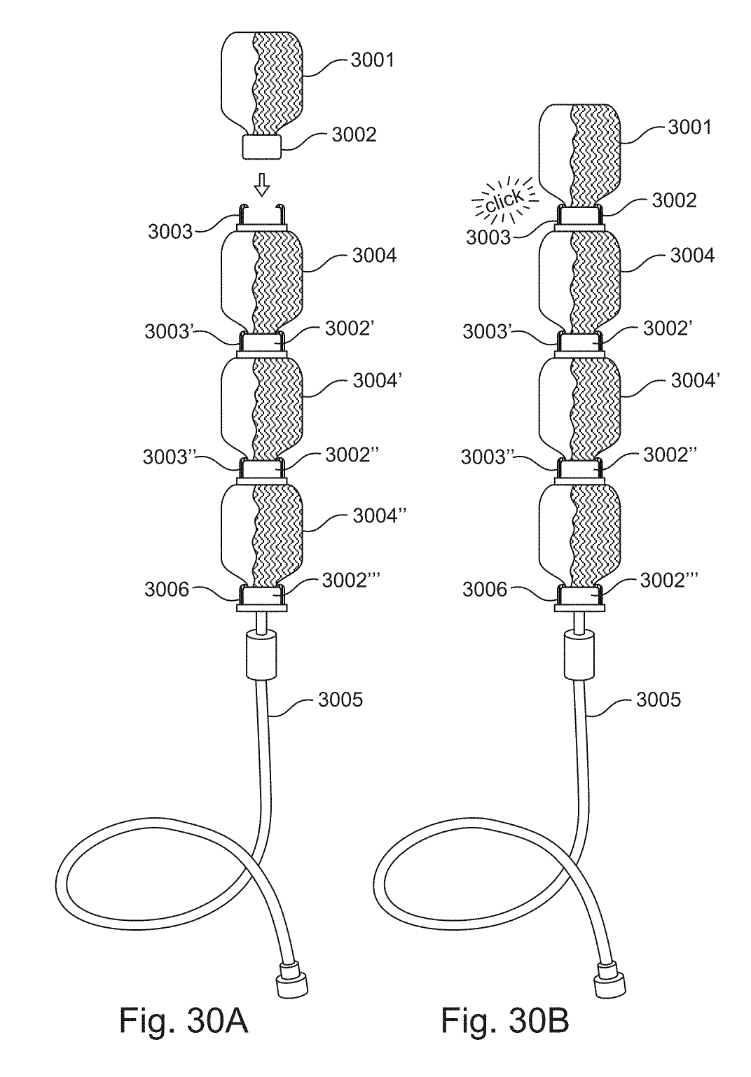

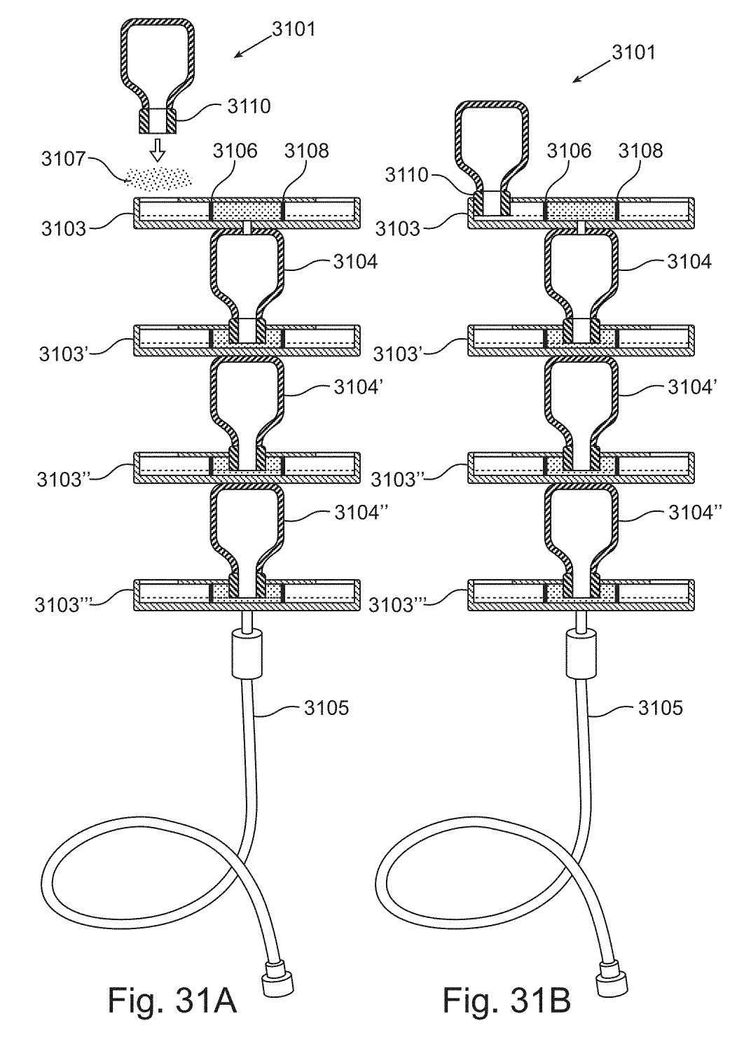

FIGS. 30A-30B are schematic front cut view illustrations presenting a further exemplary system which includes a modular dosing system of a plurality of containers 3001, 3004, whereby the containers 3001, 3004 are configured to be connected to one another, according to some embodiments of the invention.

As shown in FIG. 30A, a container 3001 is shown having a cap 3002. A second container 3004 is shown having a clasping member 3003 and a cap 3002′. The cap 3002 of the first container 3001 mates with the clasping member 3003 of the second container 3004 in order to place the container 3001 and 3004 in fluid communication with one another. Additional containers 3004′ and 3004″ are mated together in a similar series configuration terminating in IV line 3005.

FIG. 30B shows that when container 3001 is mated with container 3004, a “click” sound is provided to know when the containers are engaged and/or hermetically sealed with one another.

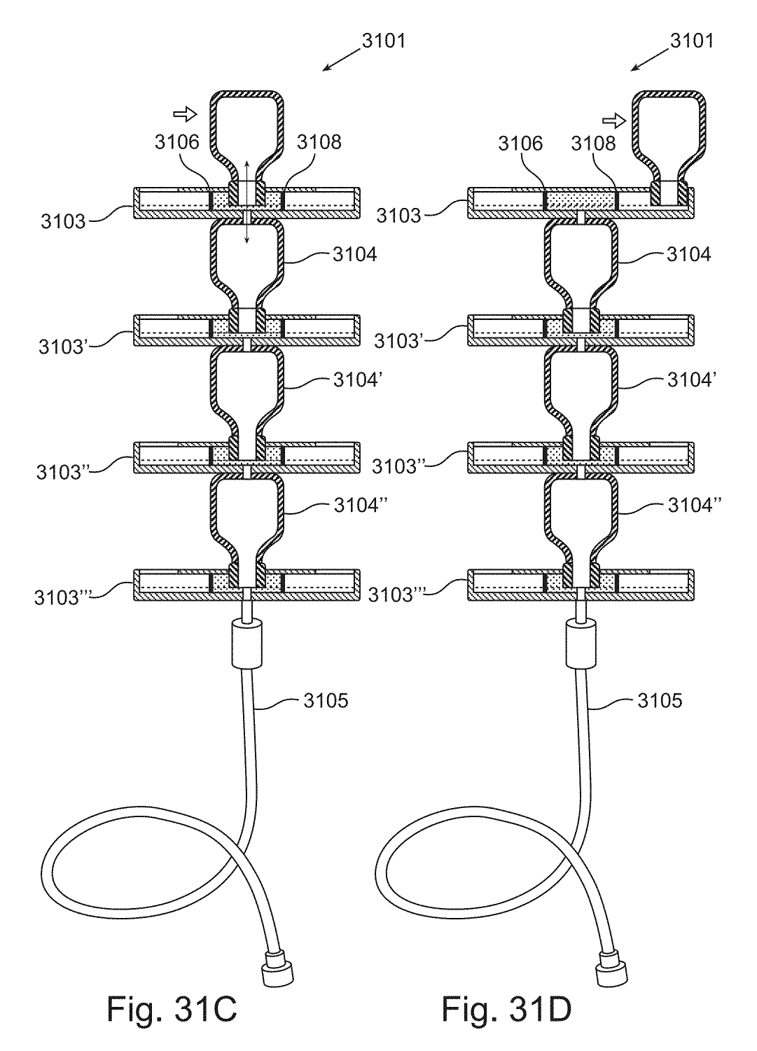

FIGS. 31A-31D are schematic front cut view illustrations presenting a further exemplary system 3101 which includes a modular dosing system of a plurality of containers 3101, 3104, whereby the containers 3101, 3104 are configured to be connected to one another, according to some embodiments of the invention.

As shown in FIG. 31A, container 3102 and entry port 3103 are separated and contaminants 3107 are shown on the distal tip of container 3102, which has a cap member 3110 with an opening. In one or more embodiments, the opening of the cap member 3110 may be sealed. In FIG. 31B, container 3102 is engaged with entry port 3103 of container 3104. The container 3102 is able to slide from a first compartment in the entry port 3103 to a second compartment in the entry port 3103 as shown in FIGS. 31B-31C. The contaminants 3107 are not able to enter the second compartment as a wiping member 3106 is provided that removes the contaminants from the surface of container 3102.

FIG. 31C shows the transfer of dose from the container 3102 through the entry port 3103 into another container 3104 through which is a contaminant-free fluid passageway in the second compartment.

After the dose is transferred, FIG. 31D shows the container 3102 sliding to a third compartment. A wiping member 3108 is used to decontaminate the distal tip of container 3102 prior to passing into the third compartment.

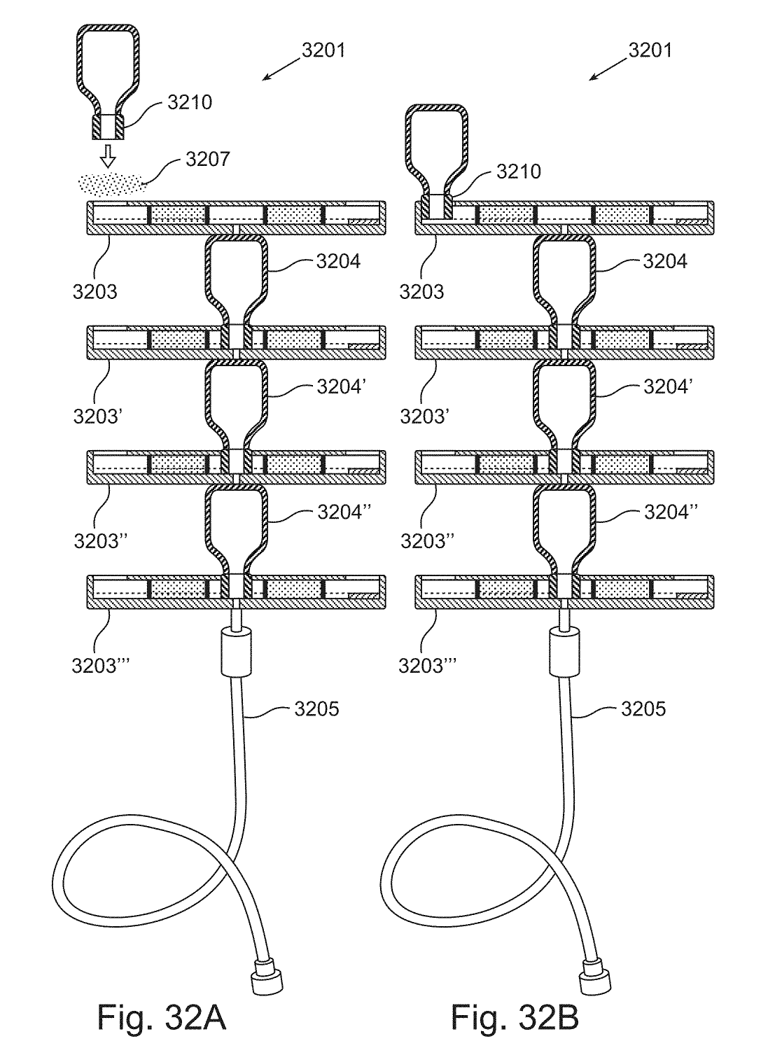

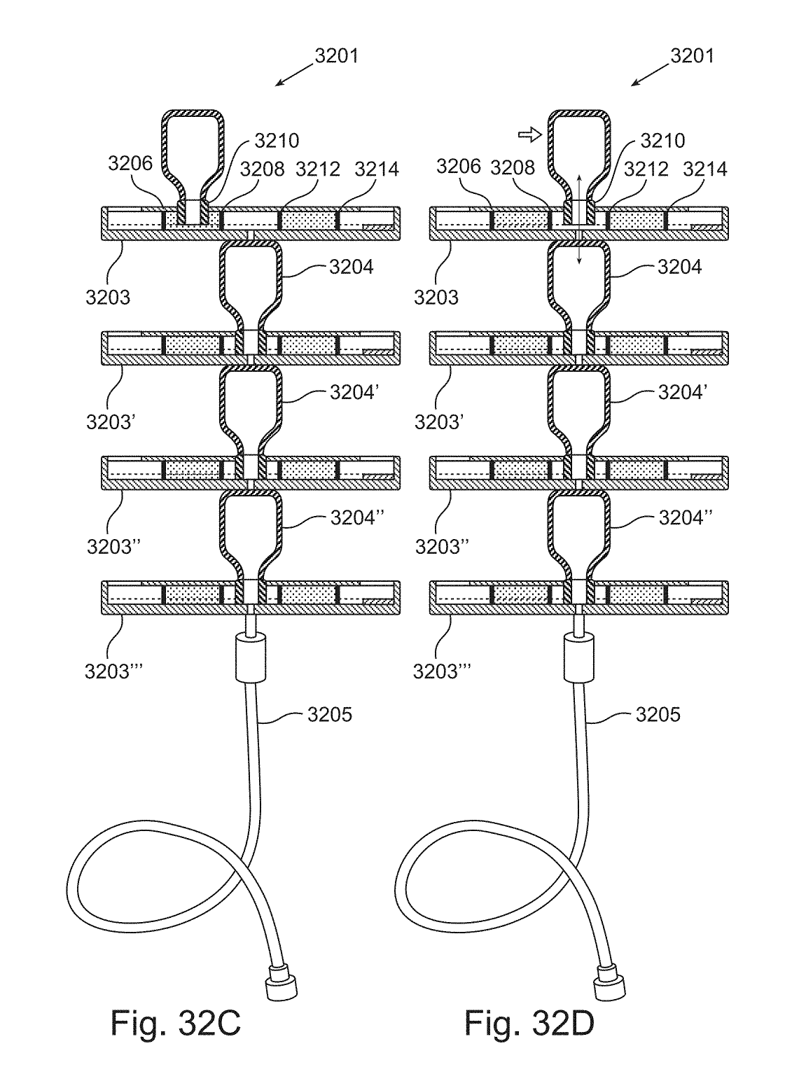

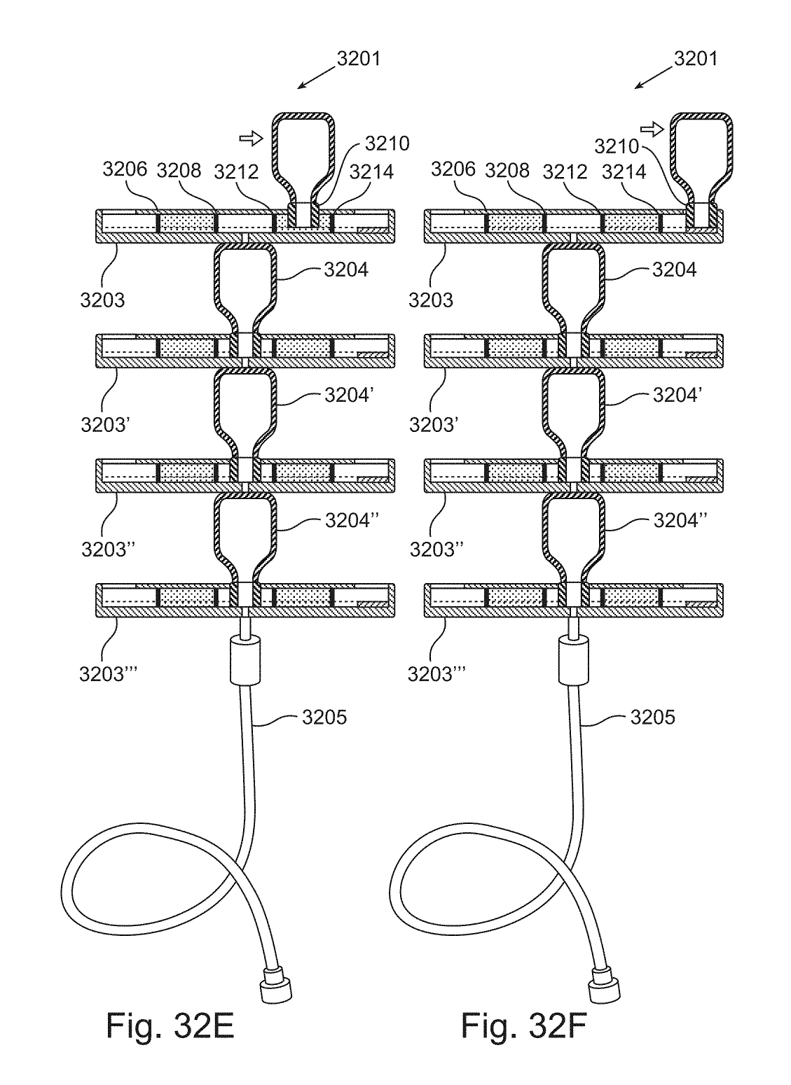

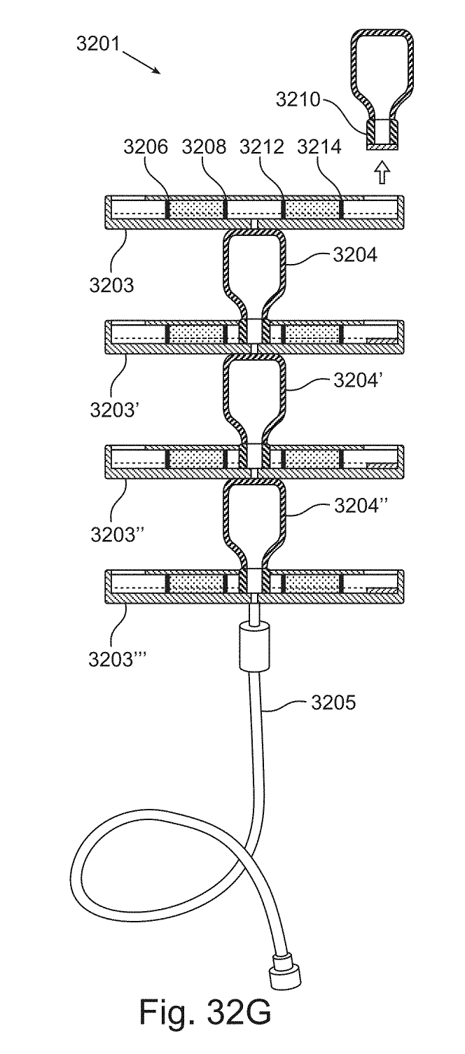

FIGS. 32A-32G are schematic front cut view illustrations presenting a further exemplary system which includes a modular dosing system of a plurality of containers 3202, 3204, whereby the containers 3202, 3204 are configured to be connected to one another, according to some embodiments of the invention.

As shown in FIG. 32A, container 3202 and entry port 3203 are separated and contaminates 3207 are shown on the distal tip of container 3202, which has a cap member 3210 with an opening, wherein the opening may be sealed or covered. In FIG. 32B, container 3302 is engaged with entry port 3203. The container 3302 is able to slide from a first compartment in the entry port 3203 to a second compartment in the entry port 3203 and as shown in FIGS. 32B-32C. The contaminants 3207 are not able to enter the second compartment as a wiping member 3206 is provided that removes the contaminants from the surface of container 3202. The container 3202 then slides from second compartment to third compartment after passing wiping member 3208. The second compartment of entry port 3203 may house a sterilizing or disinfecting substance. Optionally, the second compartment of entry port 3203 may house a sponge-like or absorptive material that wipes off the surface of container 3202. Optionally, the second compartment of entry port 3203 may have a pressure that is greater than or less than the first compartment of entry port 3203.

FIG. 32D shows the transfer of dose from the container 3202 through the entry port 3203 into another container 3204 through which is a contaminant-free fluid passageway in the third compartment.

After the dose is transferred, FIG. 32E-32F shows the container 3102 sliding from the third compartment to a fourth and fifth compartment. Wiping members 3212 and 3214 are used to decontaminate the distal tip of container 3202 prior to or at about the time of passing into the fourth and fifth compartments, respectively. The fourth compartment of entry port 3203 may house a sterilizing or disinfecting substance. Optionally, the fourth compartment of entry port 3203 may have a pressure that is greater than or less than an adjacent compartment of entry port 3203.

FIG. 32G, shows the container 3202 being removed from the entry port 3203 whereby a resealing element located in the fifth compartment allows the container 3202 to be resealed and removed.

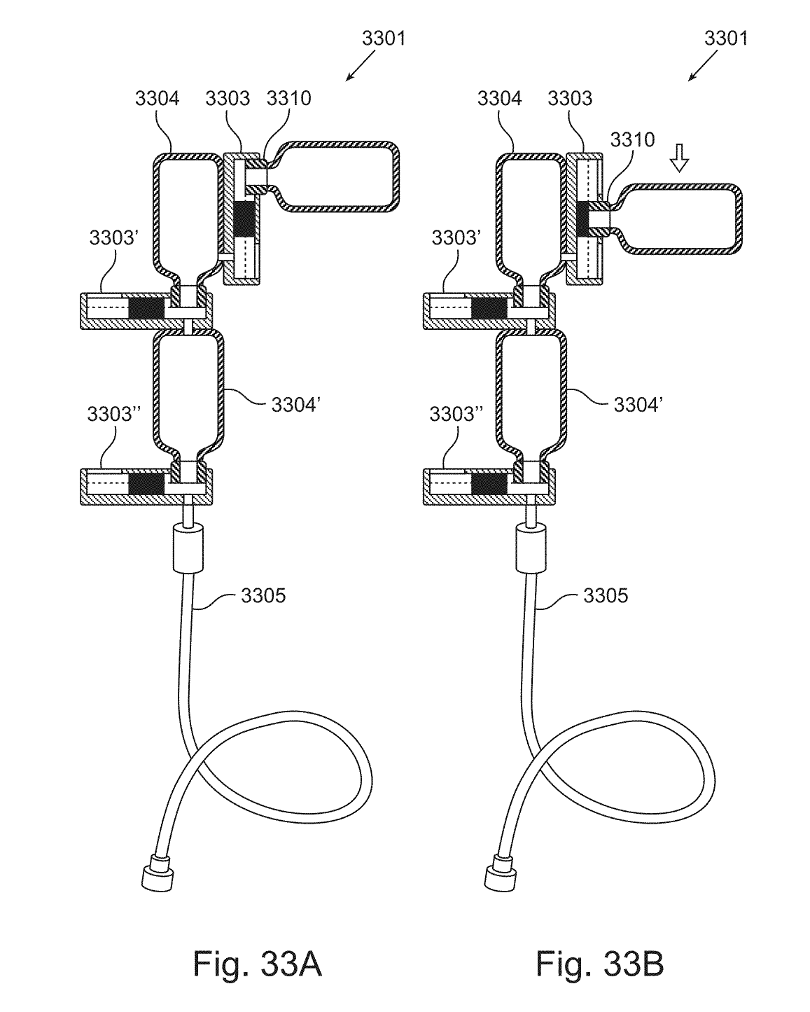

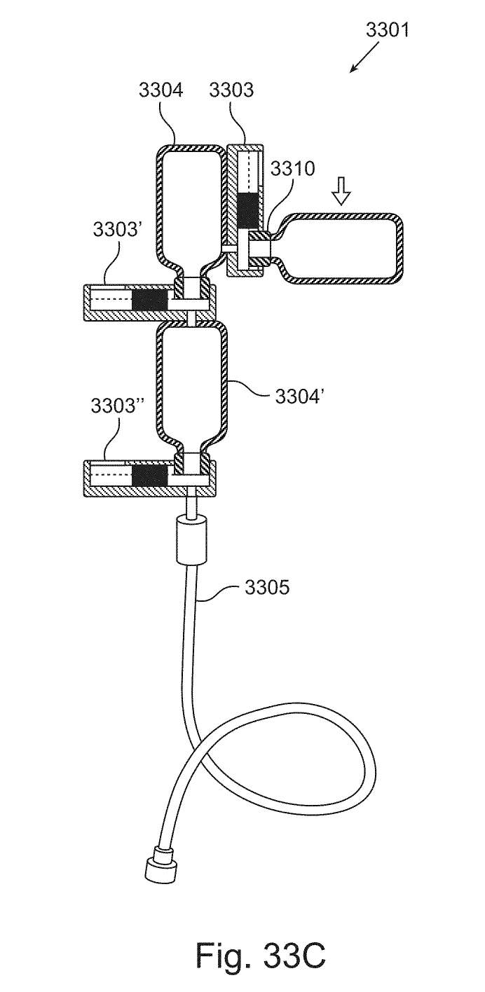

FIGS. 33A-33C are schematic front cut view illustrations presenting a further exemplary system which includes a modular dosing system of a plurality of containers 3302, 3304, whereby the containers 3302, 3304 are configured to be connected to one another, according to some embodiments of the invention.

As shown in FIG. 33A, container 3302 and entry port 3303 are engaged. Additional containers 3404 and 3304′ are also shown connected to the entry port 3303, 3303′ and 3303″ so as to form a modular system 3301, which may be connected to IV line 3305.

In FIG. 33B, container 3302 is engaged with entry port 3303. The container 3302 is able to slide from a first compartment in the entry port 3303 to a second compartment in the entry port 3303 as shown in FIG. 33B. The contaminants are not able to enter the second compartment as a wiping member is disposed between the first and second compartments that removes the contaminants from the surface of container 3302. In one or more embodiments, the wiping member is at least as wide as an opening of container 3302.

FIG. 33C shows the transfer of dose from the container 3302 through the entry port 3303 into another container 3304 through which is a contaminant-free fluid passageway in the second compartment of entry port 3303. The same operation of mechanism applies for container 3304 and container 3304′ to decontaminate the surfaces of these containers.

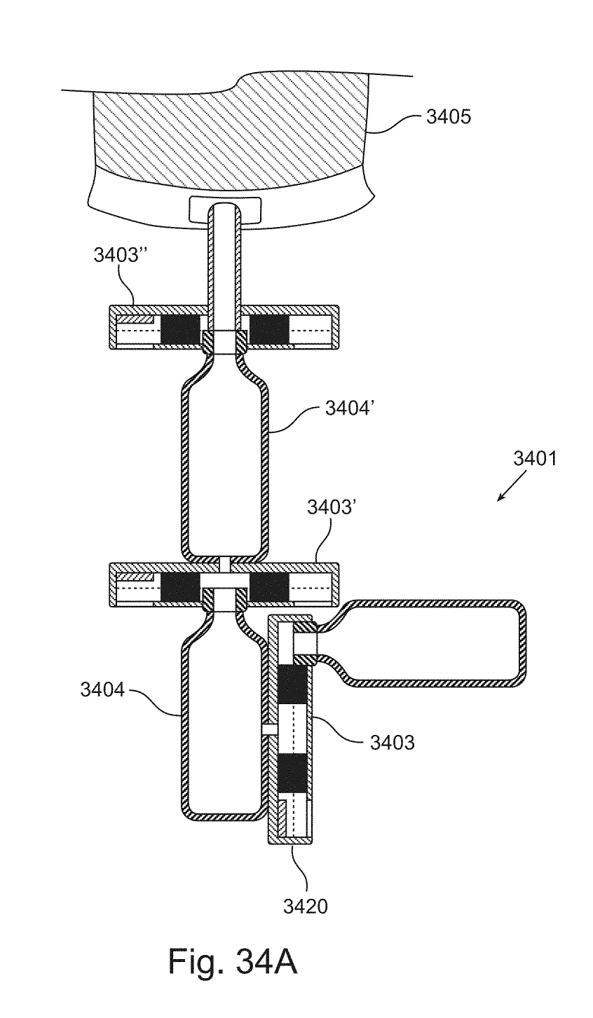

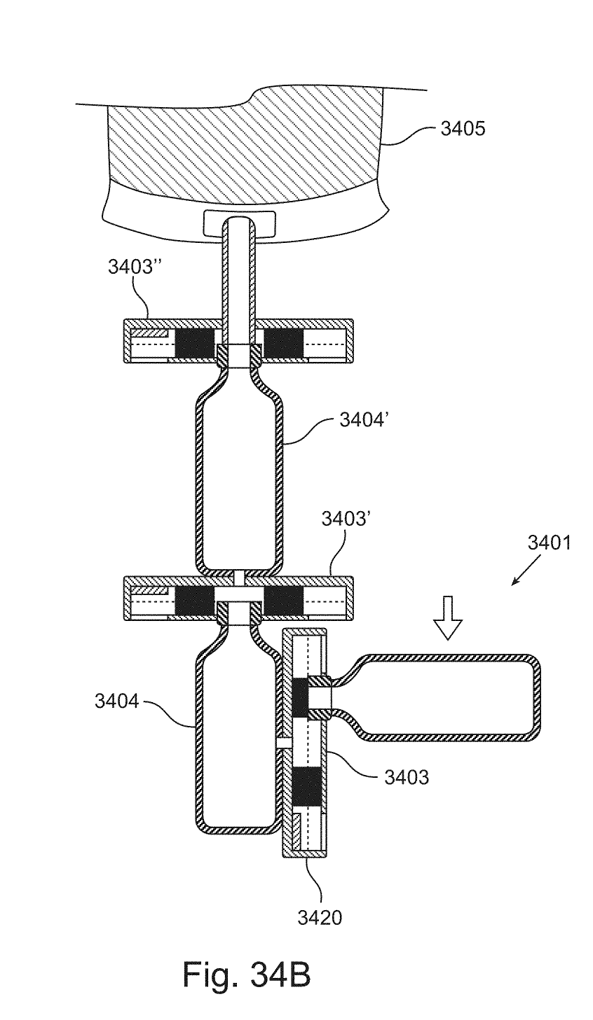

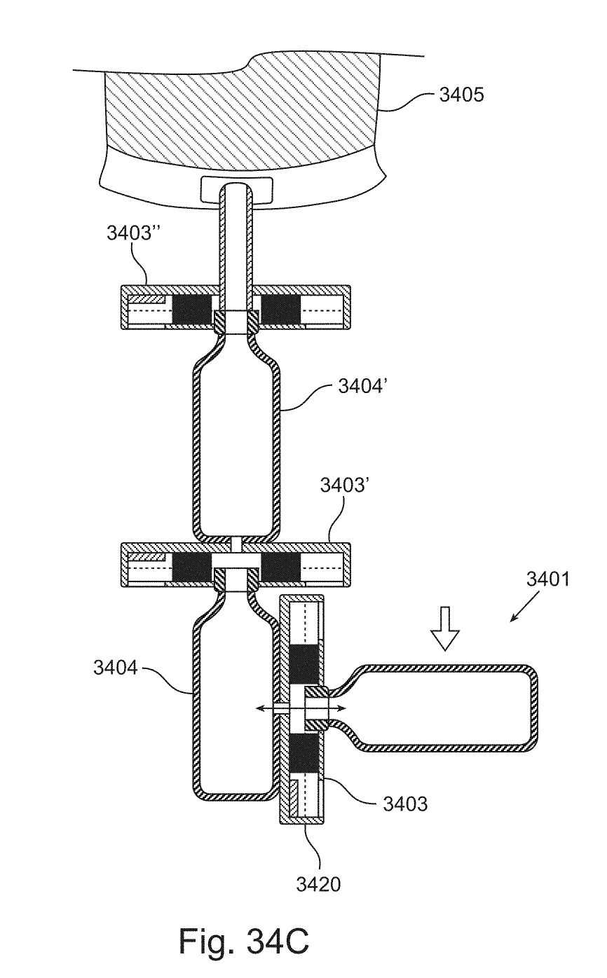

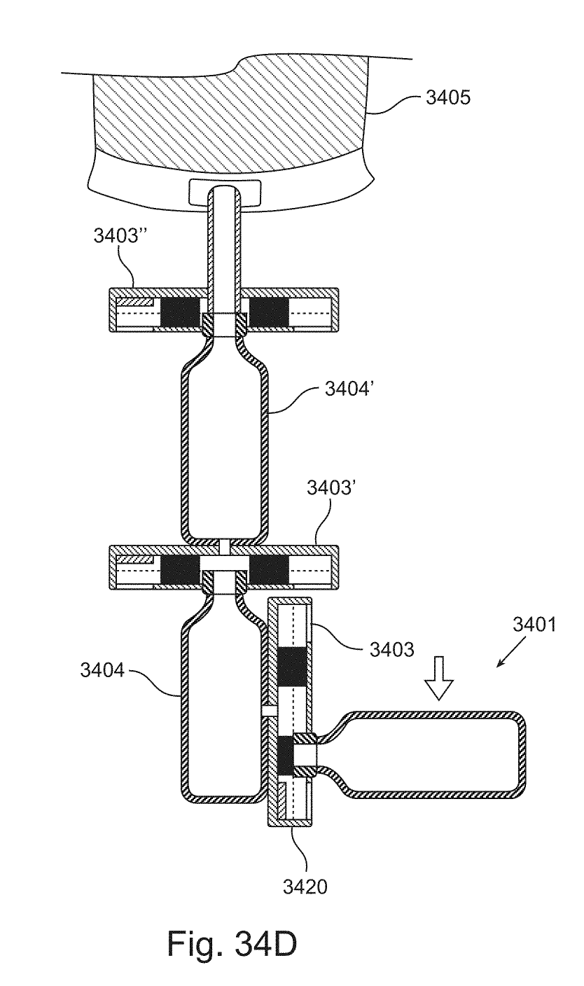

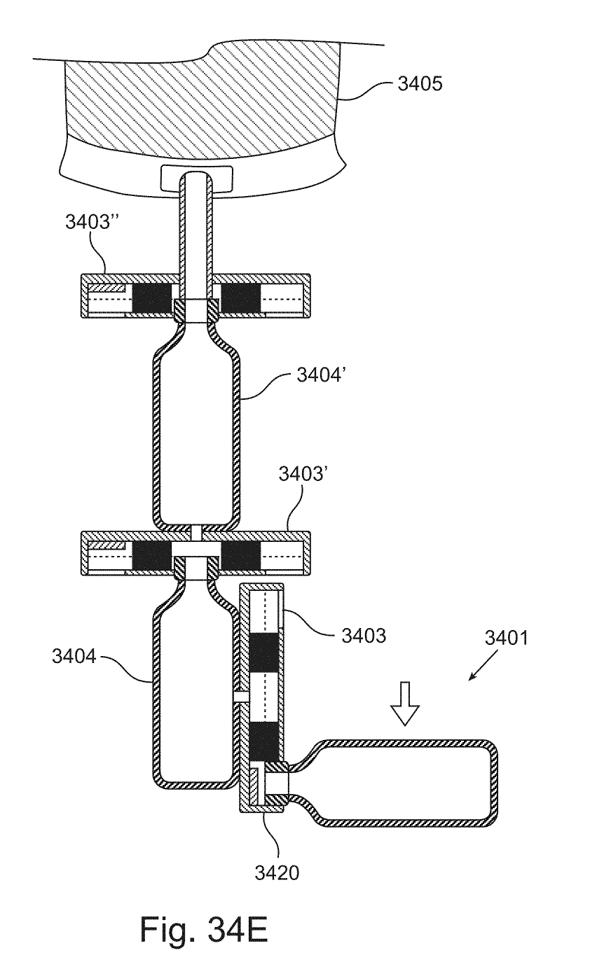

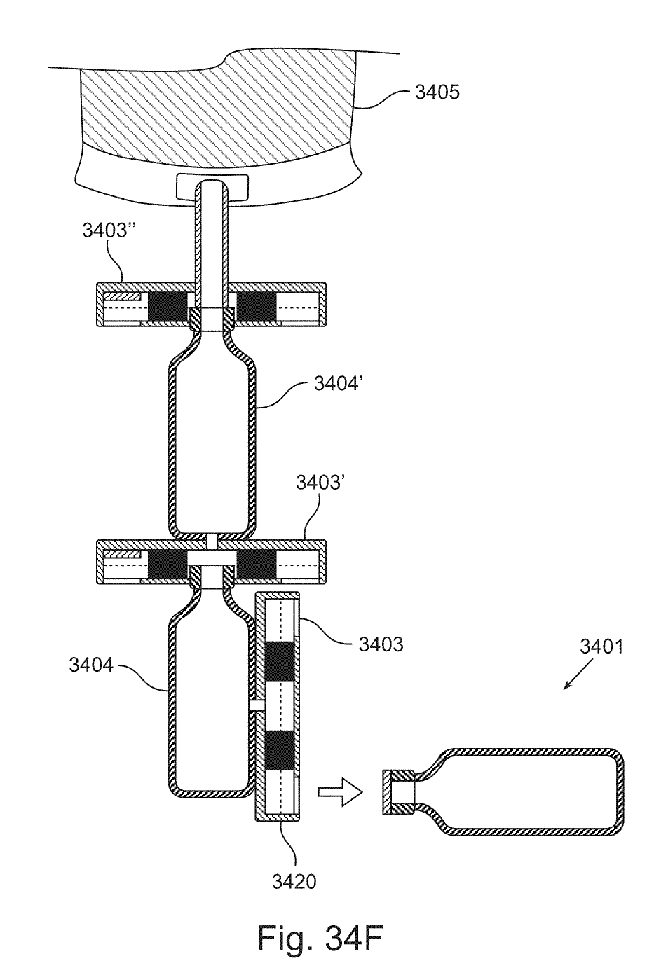

FIGS. 34A-34F are schematic front cut view illustrations presenting a further exemplary system which includes a modular dosing system of a plurality of containers 3402, 3404 connected to an IV bag 3405, whereby the containers 3404 are configured to be connected to one another, according to some embodiments of the invention.

As shown in FIG. 34A, container 3402 and entry port 3403 are engaged. The container 3402 is able to slide from a first compartment in the entry port 3403 to a second compartment in the entry port 3403 as shown in FIGS. 34B-34C. The contaminants are not able to enter the second compartment as a wiping member (black/dark rectangle) disposed between first and second compartment is provided that removes the contaminants from the surface of container 3402.

FIG. 34C shows the transfer of dose from the container 3402 through the entry port 3403 into another container 3404 through which is a contaminant-free fluid passageway in the second compartment.

After the dose is transferred, FIGS. 34D-E shows the container 3402 sliding to a third compartment. A wiping member (black/dark rectangle) disposed between the second and the third compartment is used to decontaminate the distal tip/end of container 3402 prior to passing into the third compartment. The width of the wiping member is at least as wide as an opening or aperture of container 3402.

FIG. 34F, shows the container 3402 being detached/removed from the entry port 3403 whereby a resealing element located in the third compartment reseals an opening or aperture of container 3402 and allows container 3402 to be detached/removed from entry port 3403.

The same operation of mechanism applies for container 3404 and container 3404′ to decontaminate the surfaces of these containers. Optionally, a fourth, a fifth, a sixth or more containers may be used in this modular assembly.

It is important to note that for all modular dosing systems disclosed in this invention, any vessels, containers, and/or decontamination devices/systems may have piercing members, actuators and/or frangible/rupturable seals/covers that provide for a fluidic passageway and/or a plurality of fluidic passageways to be established between the vessels, containers, and/or decontamination devices. In certain embodiments, the piercing members may be needles. In certain embodiments, the needles may be hollowed needles.

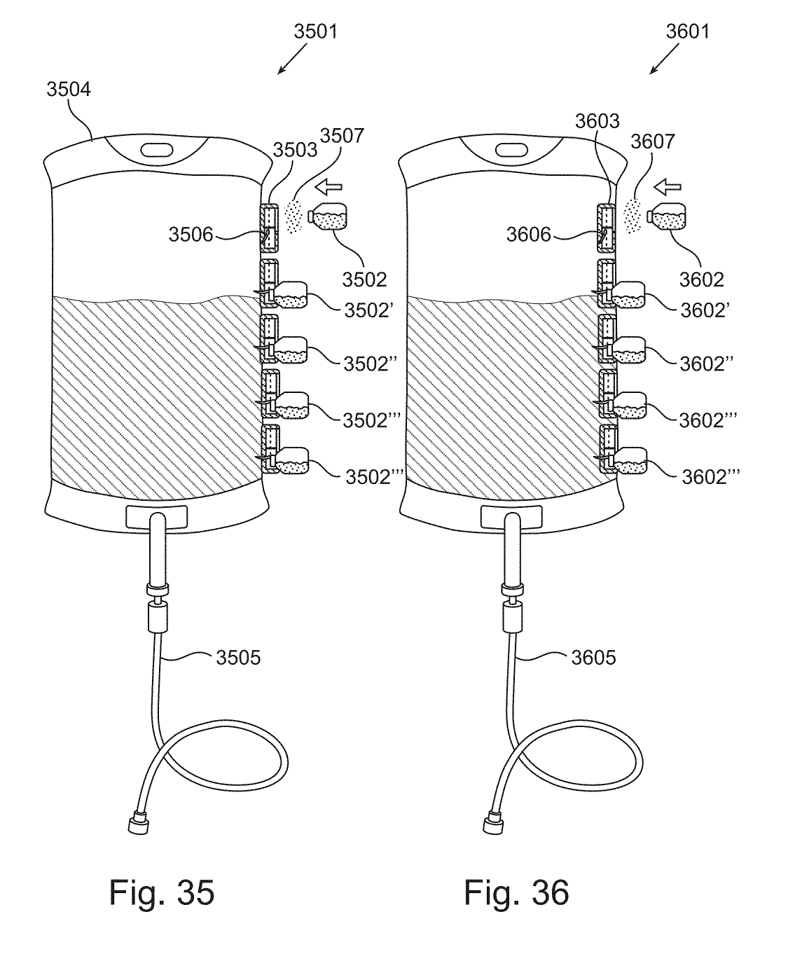

FIG. 35 is a schematic front cut view illustration presenting a further exemplary system 3501 which includes an IV bag 3504 having a plurality of entry ports 3503 and an exit port 3505, wherein a plurality of containers 3502 are received and engaged by the plurality of entry ports 3503 to transfer a dose from the plurality of containers 3502 to the IV bag 3504, according to some embodiments of the invention. It must be noted, that for any of the systems and/or devices disclosed in this invention the bags are used arbitrarily. Any vessel, device and/or container may replace the bag.

FIG. 35 shows the entry ports 3503 surface mounted onto the bag 3504 and includes an entry port 3503 having a piercing member 3506. In one or more embodiments, the entry ports 3503 abut a wall of the bag 3504. In one or more embodiments, the entry ports 3503 share a wall with the bag 3504. In one or more embodiments, the entry ports 3503 are surface mounted to a side wall of the bag 3504. In one or more embodiments, the entry ports 3503 may be located on a top wall of the bag (not shown), on a bottom wall of the bag (not shown), on a side wall of the bag and combinations thereof. In one or more embodiments, the entry ports 3503 are decontamination devices. The bag 3504 is used arbitrarily an may be any medical vessel, container and/or device. In one or more embodiments, entry ports 3503 abutting a wall of the bag may be decontamination devices. In one or more embodiments, entry ports 3503 surface mounted to a wall of a bag may be decontamination devices.

FIG. 36 is a schematic front cut view illustration presenting a further exemplary system 3601 which includes an IV bag having a plurality of entry ports 3603 and an exit port 3605, wherein a plurality of containers 3602 are received and engaged by the plurality of entry ports 3603 to transfer a dose from the plurality of containers 3602 to the IV bag 3604, according to some embodiments of the invention.

FIG. 36 shows the entry ports 3603 flush mounted within the bag and includes an entry port 3603 having a piercing member 3606. In one or more embodiments, at least a portion of the entry ports 3603 may be flush mounted within the bag. In one or more embodiments, the entire entry port may be flush mounted within the bag. In one or more embodiments, the entry ports abutting a wall of the bag. In one or more embodiments, entry ports 3603 flush mounted to the bag may be decontamination devices.

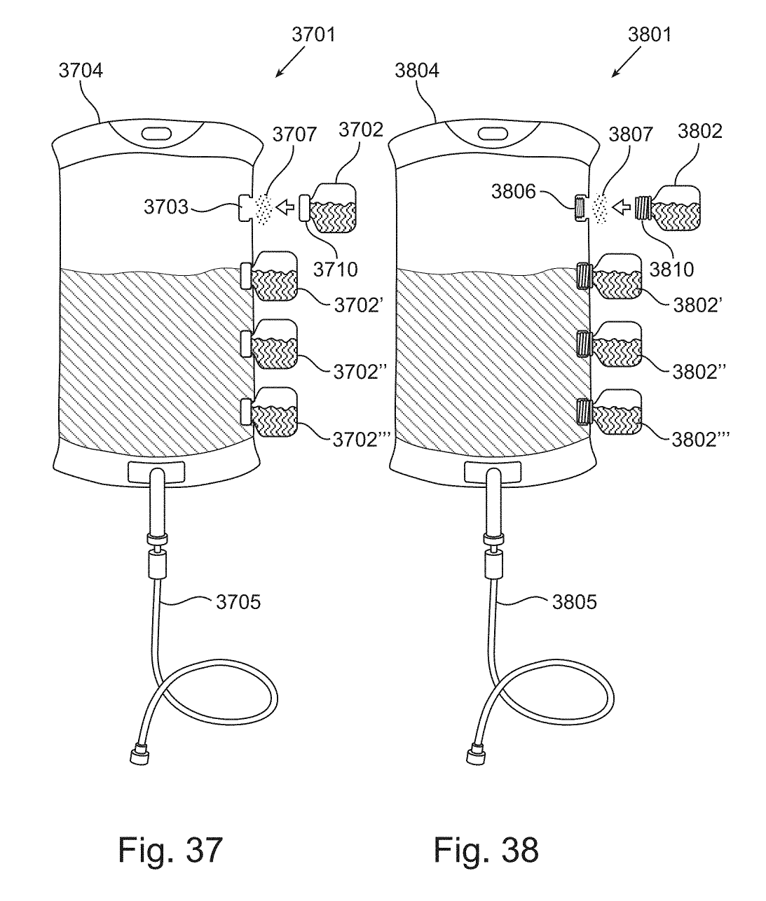

FIG. 37 is a schematic front cut view illustration presenting a further exemplary system 3701 which includes a IV bag 3704 having a plurality of entry ports 3703 and an exit port 3705, wherein a plurality of containers 3702 are received and engaged by the plurality of entry ports 3703 to transfer a dose from the plurality of containers 3702 to the IV bag 3704, according to some embodiments of the invention.

FIG. 37 shows the entry port 3703 abutting the bag 3704 whereby the container 3702 is pressed onto the bag 3704 when the container and the bag are engaged to one another. In one or more embodiments, the entry ports 3703 are flush mounted to the bag. In one or more embodiments, the containers 3702 abut a wall of the bag 3704 when the containers 3702 are engaged to the bag 3704.

FIG. 38 is a schematic front cut view illustration presenting a further exemplary system 3801 which includes an IV bag 3804 having a plurality of entry ports 3803 and an exit port 3805, wherein a plurality of containers 3802 are received and engaged by the plurality of entry ports 3803 to transfer a dose from the plurality of containers 3802 to the IV bag 3804, according to some embodiments of the invention.

FIG. 38 shows the plurality of containers 3802 having threads 3810 and the bag 3804 having a corresponding thread 3806. Contaminants 3807 are pressed between threads 3810 and 3806 and fluid can pass through a passageway between the thread members. In one or more embodiments, the threads 3806 and 3810 may be a luer. In one or more embodiments, the threads 3806 and 3810 may be luer-lock. In one or more embodiments, the threads 3806 and 3810 may be a smart-site. In one or more embodiments threads 3806 and/or threads 3810 may have a locking feature/member. In one or more embodiments, the locking feature/member may provide for a permanent locking of threads 3806 and 3810. In one or more embodiments, the permanent locking may be an irreversible locking.

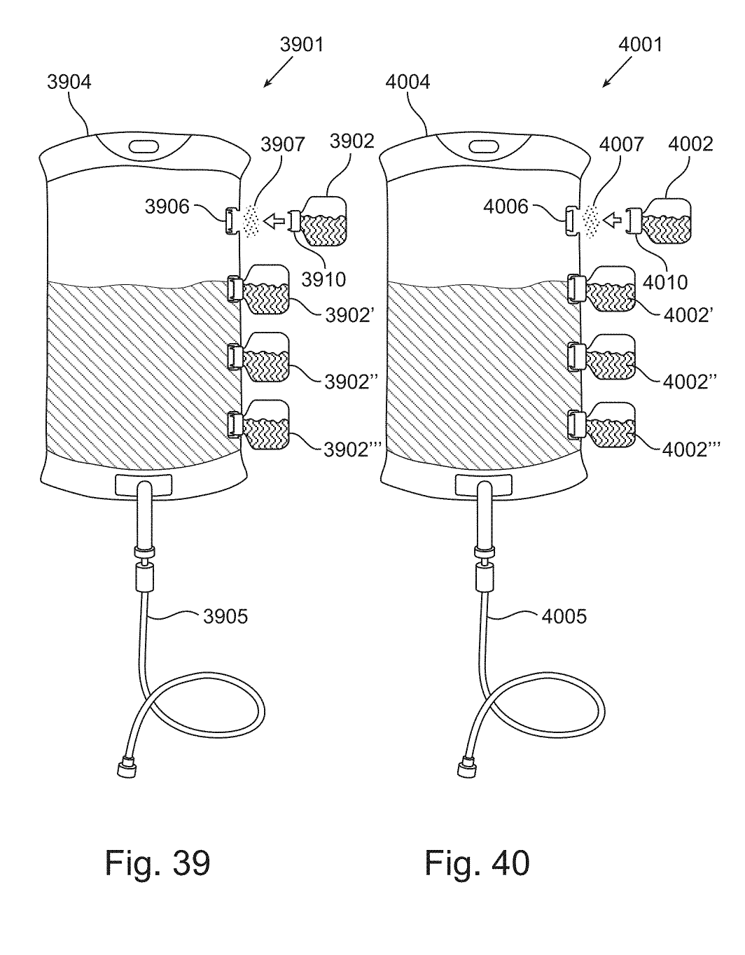

FIG. 39 is a schematic front cut view illustration presenting a further exemplary system 3901 which includes an IV bag 3904 having a plurality of entry ports 3903 and an exit port 3905, wherein a plurality of containers 3902 are received and engaged by the plurality of entry ports 3903 to transfer a dose from the plurality of containers 3902 to the IV bag 3904, according to some embodiments of the invention.

FIG. 39 shows the plurality of containers 3902 having ratchet teeth 3910 and the bag 3904 having a corresponding female member 3906 engaged by ratchet teeth 3910. Contaminants 3907 are pressed between ratchet teeth 3910 and female member 3906 and fluid can pass through a passageway between these members.

FIG. 40 is a schematic front cut view illustration presenting a further exemplary system 4001 which includes an IV bag 4004 having a plurality of entry ports and an exit port 4005, wherein a plurality of containers 4002 are received and engaged by the plurality of entry ports 4003 to transfer a dose from the plurality of containers 4002 to the IV bag 4004, according to some embodiments of the invention.

FIG. 40 shows the plurality of containers 4002 having ratchet teeth 4010 and the bag 4004 having a corresponding female member 4006 disposed on or within the entry ports engaged by ratchet teeth 4010. Contaminants 4007 are pressed between ratchet teeth 4010 and female member 4006 and fluid can pass through a passageway between these members. The female members 4006 are flush mounted to the bag. Optionally, a plurality of the female members abut a wall of the bag.

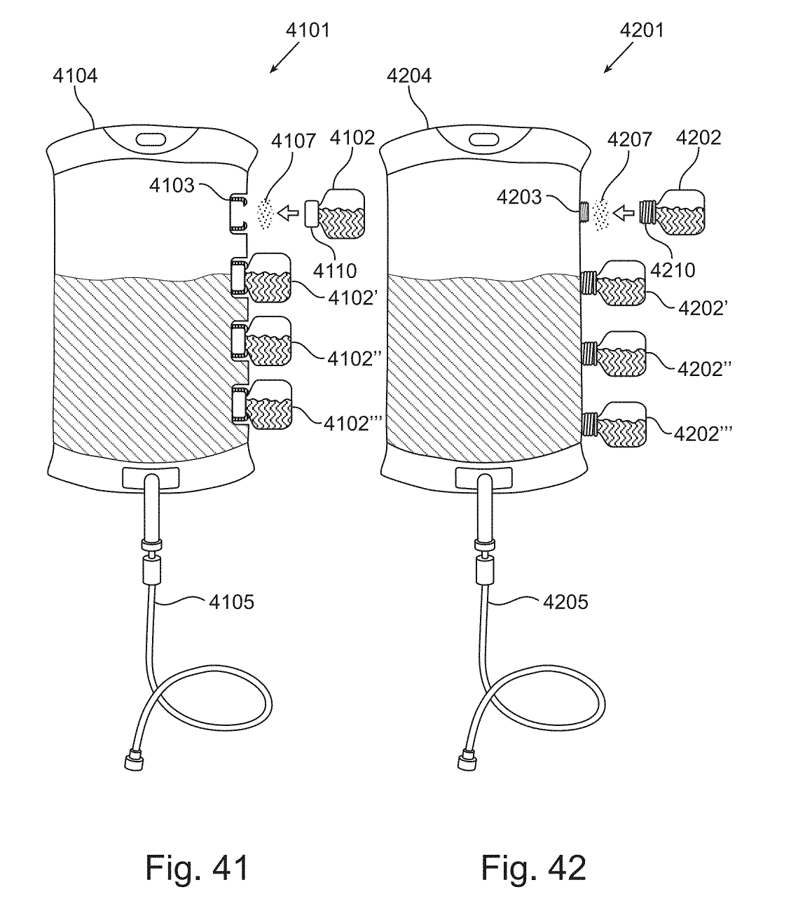

FIG. 41 is a schematic front cut view illustration presenting a further exemplary system 4101 which includes an IV bag 4104 having a plurality of entry ports 4103 and an exit port 4105, wherein a plurality of containers 4102 are received and engaged by the plurality of entry ports 4103 to transfer a dose from the plurality of containers 4102 to the IV bag 4104, according to some embodiments of the invention.

FIG. 41 shows the plurality of containers 4102 having a cap 4110 and the bag 4104 having a clasping member 4103 that engages cap 4110. Contaminants 4107 are pressed between cap 4110 and clasping member 4103 and fluid can pass through a passageway between these members. The clasping member 4103 is flush mounted to the bag 4104. Optionally, the clasping member 4103 abuts a wall of the bag. In one or more embodiments, the bag 4104 has a plurality of piercing members (not shown) configured to pierce a surface of at least cap 4110 to establish a fluidic communication between containers 4102 and bag 4104. In one or more embodiments, containers 4102 have piercing members configured to pierce a surface/cap 4110 of containers 4102 at about the time or after engagement with bag 4104. In one or more embodiments, both bag 4104 and containers 4102 have piercing member configured to pierce at least one surface of bag 4104 and container 4102 to establish a fluidic passageway between bag 4104 and container 4102. In one or more embodiments, the piercing members are disposed within bag 4104. In one or more embodiments, the piercing members are disposed within containers 4102.

FIG. 42 is a schematic front cut view illustration presenting a further exemplary system 4201 which comprises an IV bag 4204 having a plurality of threads 4203 and an exit port 4205, wherein a plurality of containers 4202 are received and engaged by the plurality of threads 4203 to transfer a dose from the plurality of containers 4202 to the IV bag 4204, according to some embodiments of the invention.

FIG. 42 shows the plurality of containers 4202 having a thread 4210 and the bag 4204 having a plurality of thread 4203 that engages thread 4210. Contaminants 4207 are pressed between thread 4210 and thread 4203 and fluid can pass through a passageway between these members. The thread 4203 is flush mounted to the bag 4204. Optionally, the threads 4203 abut the bag. Optionally, the threads 4203 abut a wall of the bag.

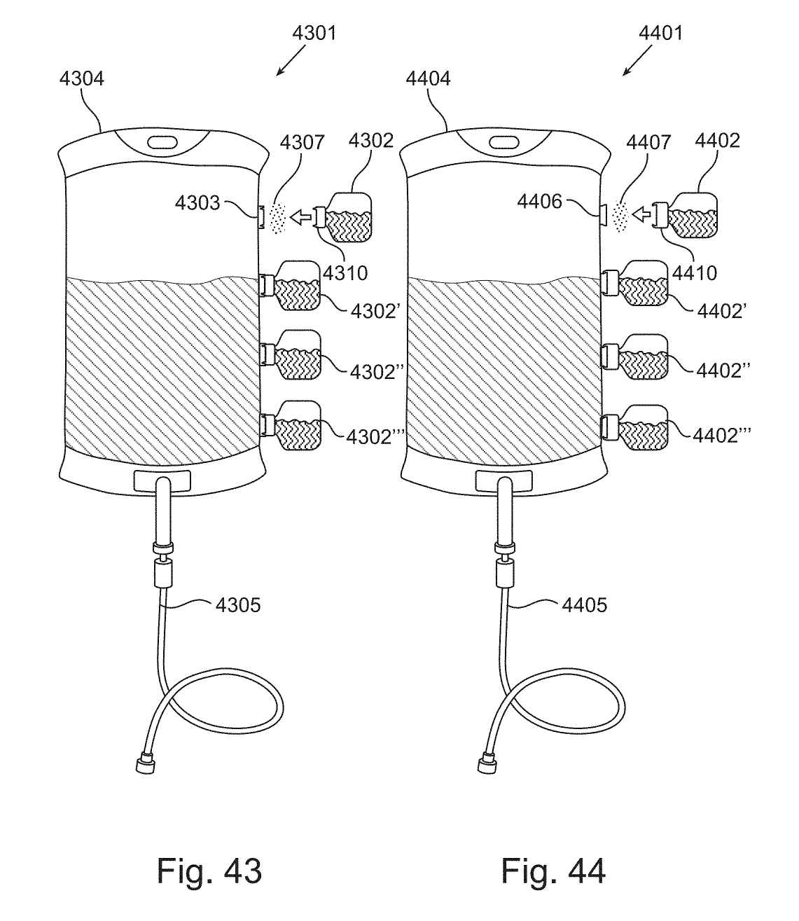

FIG. 43 is a schematic front cut view illustration presenting a further exemplary system 4301 which includes an IV bag 4304 having a plurality of female members 4303 and an exit port 4305, wherein a plurality of containers 4302 are received and engaged by the plurality of female members 4303 to transfer a dose from the plurality of containers 4302 to the IV bag 4304, according to some embodiments of the invention.

FIG. 43 shows the plurality of containers 4302 having a ratchet tooth 4310 and the bag 4304 having a plurality of female members 4303 that engage ratchet teeth 4310. Contaminants 4307 are pressed between ratchet teeth 4310 and female members 4303 and fluid can pass through a passageway between these members. The female members 4303 are surface mounted to the bag 4304. Optionally, the female members 4303 abut a wall of the bag.

FIG. 44 is a schematic front cut view illustration presenting a further exemplary system 4401 which includes an IV bag 4404 having a plurality of entry ports and an exit port 4405, wherein a plurality of containers 4402 are received and engaged by the plurality of entry ports to transfer a dose from the plurality of containers 4402 to the IV bag 4404, according to some embodiments of the invention.

FIG. 44 shows the plurality of containers 4402 having a ratchet tooth 4410 and the bag 4404 having a plurality of entry ports (male members) 4406 that engage ratchet teeth 4410. Contaminants 4407 are pressed between ratchet teeth 4410 and entry ports (male members) 4406 and fluid can pass through a passageway between these members. The entry ports (male members) 4406 are surface mounted to the bag 4404. Optionally, the entry ports (male members) 4406 abut a wall of the bag 4404.

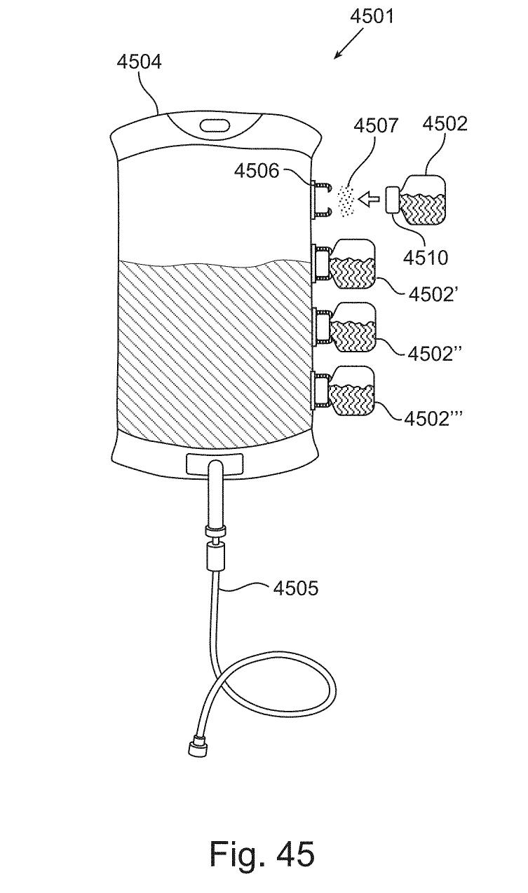

FIG. 45 is a schematic front cut view illustration presenting a further exemplary system 4501 which includes an IV bag 4504 having a plurality of entry ports and an exit port 4505, wherein a plurality of containers 4502 are received and engaged by the plurality of entry ports to transfer a dose from the plurality of containers 4502 to the IV bag 4504, according to some embodiments of the invention.

FIG. 45 shows the plurality of containers 4502 having a cap 4510 and the bag 4504 having a plurality of entry ports with clasping members 4506 that engage caps 4510. Contaminants 4507 are pressed between cap 4510 and entry ports with clasping members 4506 and fluid can pass through a passageway between these members. The entry ports with clasping members 4506 are flush mounted to the bag 4504. The clasping members 4506 may abut a wall of the bag.

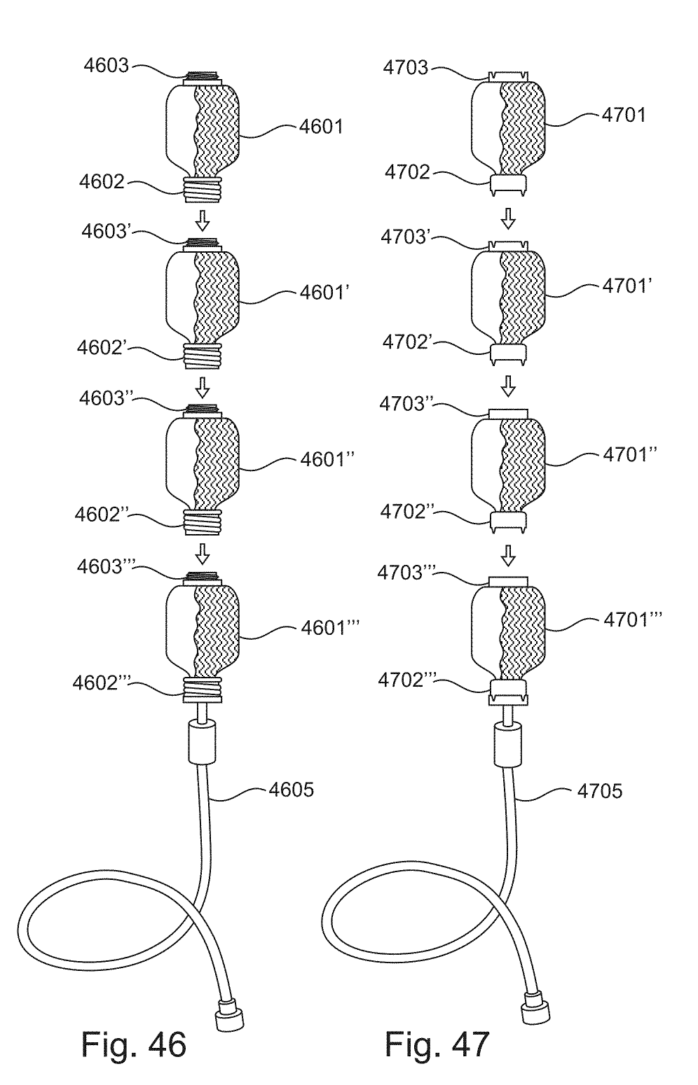

FIG. 46 is a schematic front cut view illustration presenting a further exemplary system which comprises a modular dosing system of a plurality of containers 4601, whereby the containers 4601 are configured to be connected to one another, according to some embodiments of the invention.

As shown in FIG. 46, a container 4601 is shown having lower thread 4602 and upper thread 4603. A second container 4601′ is shown having an upper thread 4603′ and a lower thread 4602′. The lower thread 4602 of the first container 4601 mates with the upper thread 4603′ of the second container 4601′ in order to place the container 4601 and 4601′ in fluid communication with one another. Additional containers 4601″ and 4601′″ are mated together in a similar series configuration terminating in IV line 4605. In one or more embodiments, any of containers 4601, optionally a plurality of containers 4601, may have at least one piercing member or a displaceable container surface providing for at least one fluidic passageway between the containers.

As shown in FIG. 47, a container 4701 is shown having lower ratchet teeth 4702 and upper ratchet teeth 4703. A second container 4701′ is shown having an upper ratchet tooth 4703′ and a lower ratchet teeth 4702′. The lower ratchet teeth 4702 of the first container 4701 mates with the upper ratchet teeth 4703′ of the second container 4701′ in order to place the container 4701 and 4701′ in fluid communication with one another. Additional containers 4701″ and 4701′″ are mated together in a similar series configuration terminating in IV line 4705.

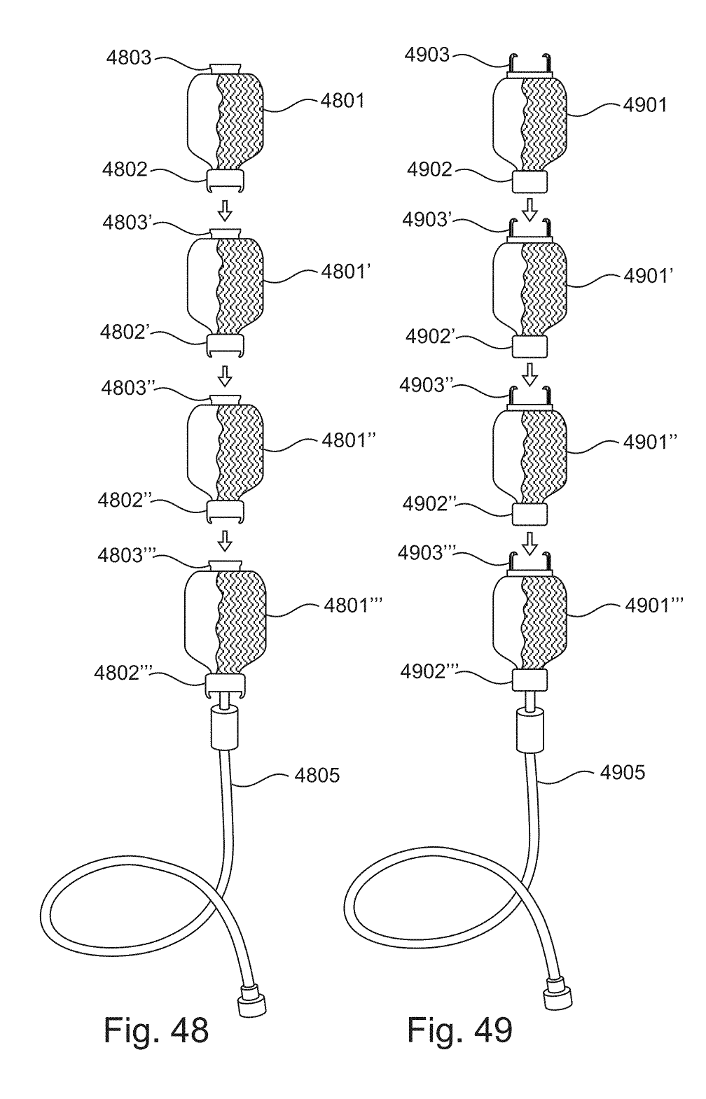

As shown in FIG. 48, a container 4801 is shown having receiving member 4803 and lower ratchet teeth 4802. A second container 4801′ is shown having an receiving member 4803′ and a lower ratchet teeth 4802′. The lower ratchet teeth 4802 of the first container 4801 mates with the receiving member 4803′ of the second container 4801′ in order to place the container 4801 and 4801′ in fluid communication with one another. Additional containers 4801″ and 4801′″ are mated together in a similar series configuration terminating in IV line 4805.

As shown in FIG. 49, a container 4901 is shown having clasping member 4903 and cap 4902. A second container 4901′ is shown having a clasping member 4903′ and cap 4902′. The cap 4902 of the first container 4901 mates with the clasping member 4903′ of the second container 4901′ in order to place the container 4901 and 4901′ in fluid communication with one another. Additional containers 4901″ and 4901′″ are mated together in a similar series configuration terminating in IV line 4905. It must be noted that for all modular systems the engagement between containers may be airtight or hermetic.

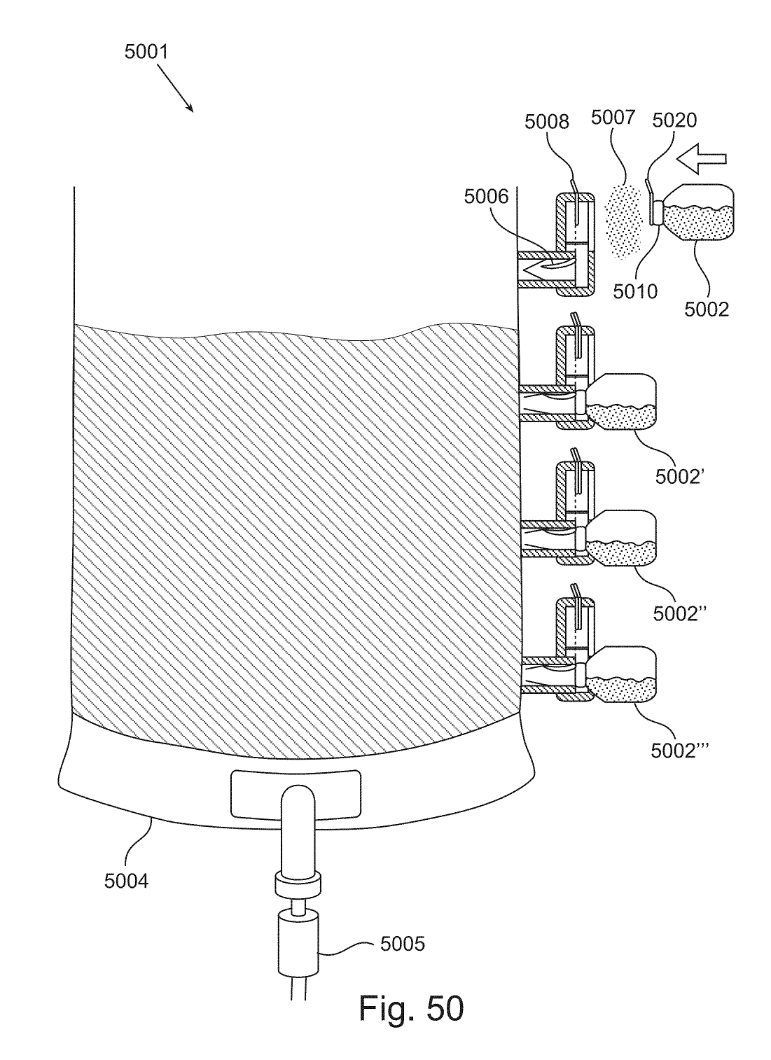

FIG. 50 is a schematic front cut view illustration presenting a further exemplary system 5001 which includes an IV bag 5004 having a plurality of entry ports and an exit port 5005, showing a plurality of containers 5002 able to be received by the plurality of entry ports 5002, according to some embodiments of the invention.

FIG. 50 shows the plurality of containers 5002 having a cap 5010 and the bag 5004 having an entry port 5006 that engages cap 5010. Contaminants 5007 are pressed between entry port 5006 and cap 5010. Optionally, cap 5010 and container 5002 once engaged with entry port 5006 slides from a first position to a second position. The cap has an decontamination interface 5020 and the entry port has a decontamination interface 5008 which mate with one another and are configured to slide externally as the cap 5010 is displaced from a first position to a second position.

In the second position, the container 5002 is in fluid communication with the bag 5004 through entry port 5006. In certain embodiments, a piercing member is used to pierce the cap 5010. The piercing member may be disposed within the port of bag 5004 or within a chamber of bag 5004.

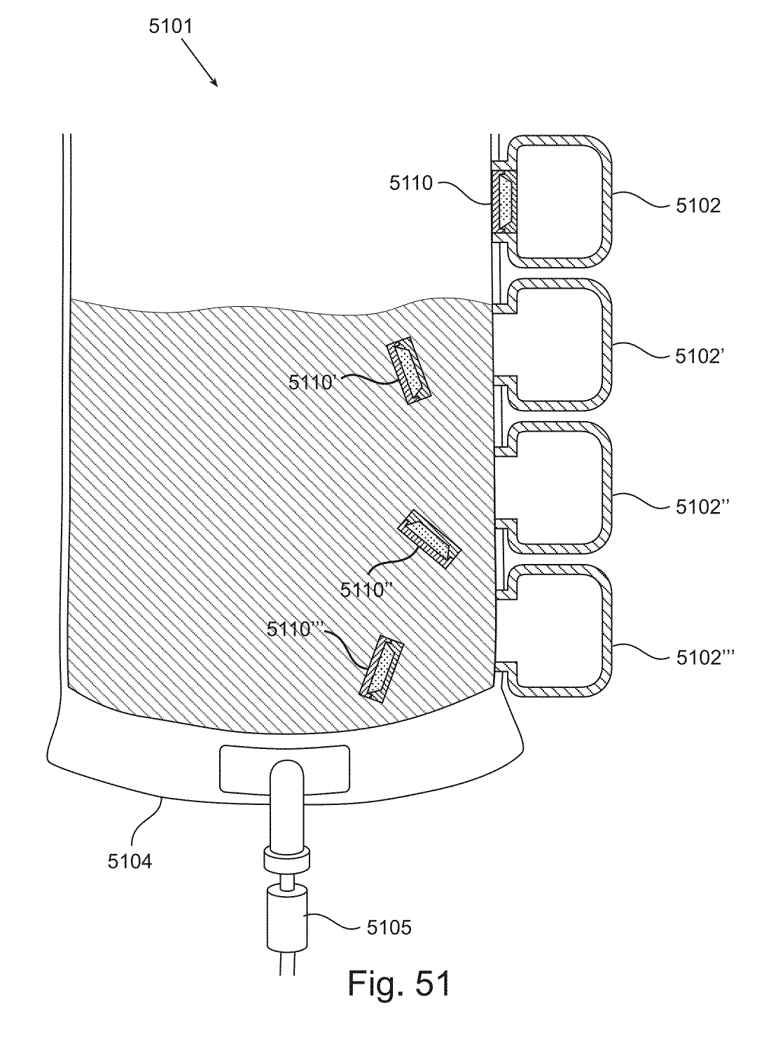

FIG. 51 is a schematic front cut view illustration presenting a further exemplary system 5101 which includes an IV bag 5104 having a plurality of entry ports and an exit port 5105, showing a plurality of containers 5002 able to be received by the plurality of entry ports 5103, according to some embodiments of the invention.

FIG. 51 shows the plurality of containers 5102 having a cap that mates with a plurality of entry ports 5110 of the bag. Contaminants are pressed between entry ports of the bag 5110 and the Caps. The cap and container 5102 once engaged with entry port 5110 displace internally into the bag 5104. Contaminants 5107 are housed within or between the interface between the entry port cap 5110 and the cap of container 5102.

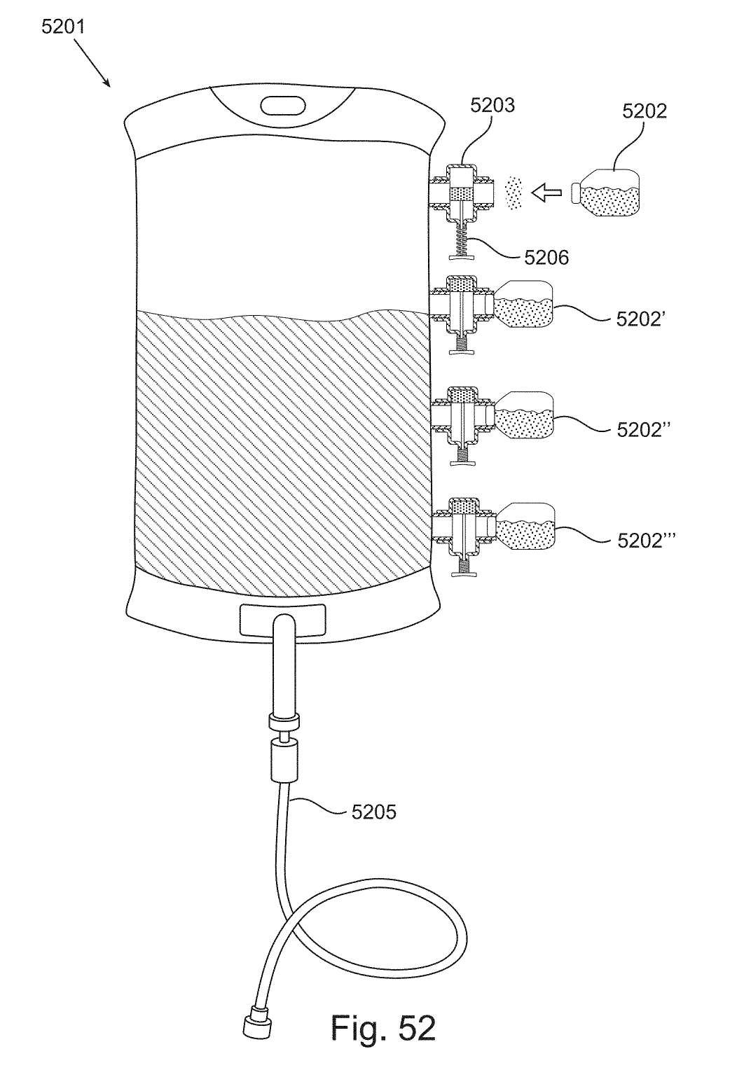

FIG. 52 is a schematic front cut view illustration presenting a further exemplary system which includes an IV bag 5201 having a plurality of entry ports 5203 and an exit port 5205, showing a plurality of containers 5202 able to be received by the plurality of entry ports 5203, according to some embodiments of the invention.

FIG. 52 shows the entry ports 5203 having a wiping member configured to move across the housing to decontaminate a surface of the plurality of containers 5002. In one or more embodiments, the entry ports are decontamination devices.

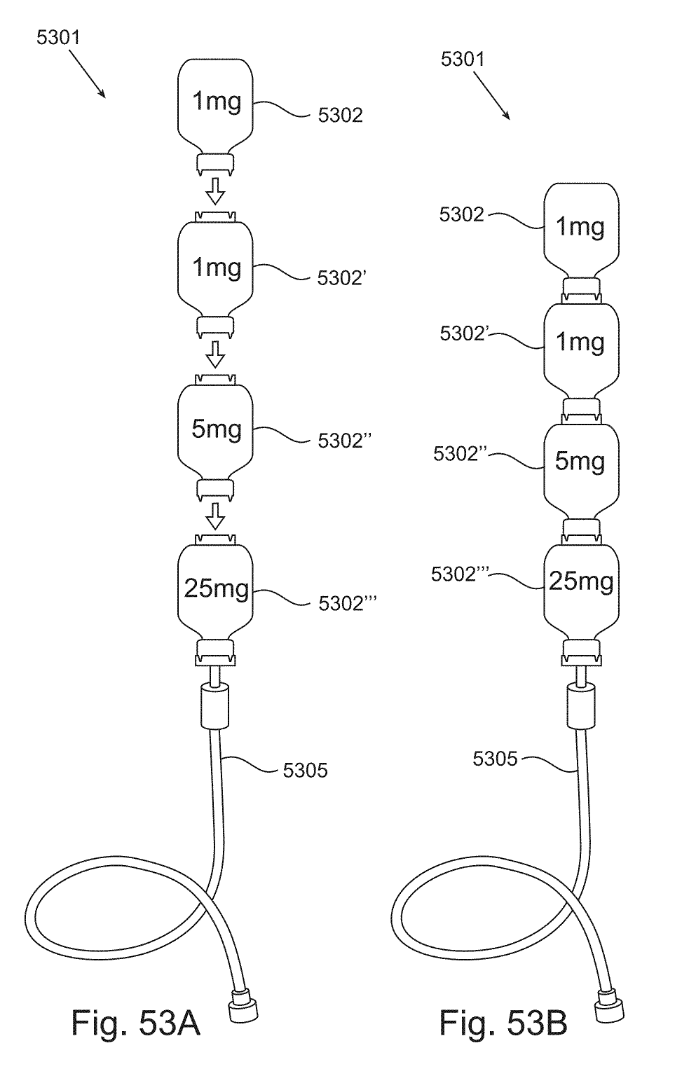

FIGS. 53A-53B is a schematic front cut view illustration presenting a further exemplary system which includes a modular dosing system of a plurality of containers 5302 showing amounts of a beneficial substance housed in the containers 5302, whereby the containers 5302 are configured to be connected to one another, according to some embodiments of the invention. In one or more embodiments, a plurality of the containers 5302 are in a fluidic communication each other at about the time and/or after engagement of the plurality of the containers.

In the above referenced figures and descriptions, the plurality of containers and/or ports and components incorporate by reference the teachings of U.S. application Ser. Nos. 16/100,594; 16/100,712; 16/100,840; 16/100,964.

As shown in FIG. 53A, a container 5302 is shown having ratchet teeth. A second container 5302′ is shown having an upper ratchet teeth and a bottom ratchet teeth. The ratchet teeth of the first container 5302 mates with the upper ratchet teeth of the second container 5302′ in order to place the containers in fluid communication, and optionally, in an airtight engagement with one another. The containers can have different amounts of the same or a different beneficial substance, such as 1 mg, 5 mg, 25 mg or other amounts, so that a user may assemble and/or prepare a customized final amount of a beneficial substance to a patient or animal. FIG. 53A shows the modular system 5301 in an open configuration, while FIG. 53B shows the modular system 5301 in a connected configuration. In one or more embodiments, upon engagement of the plurality of containers 5302 the containers 5302 are in a fluidic communication with each other and the beneficial substance and/or drug housed in the containers is pulled down into the infusion line via a force of gravity. In one or more embodiments, at least one of the containers 5302 houses a non-standard amount of a beneficial substance and/or drug. In one or more embodiments, a plurality of the containers 5302 houses a non-standard amount of a beneficial substance and/or a drug. Packaging non-standard amounts of a beneficial substance and/or a drug in commercially prepackaged containers 5302 allows for the assembly of a customized and/or individualized amount of a beneficial substance and/or a drug without the need to manually measure and manipulate the beneficial substance. In one or more embodiments, at least one of the containers 5302 houses a drug in an amount less than about 5% of a Table 1 Column A or Table 2 Column A amount. In one or more embodiments, at least one of the containers 5302 houses a drug in an amount less than about 10% of a Table 1 Column A or Table 2 Column A amount. In one or more embodiments, at least one of the containers 5302 houses a drug in an amount less than about 20% of a Table 1 Column A or Table 2 Column A amount. In one or more embodiments, at least one of the containers 5302 houses a drug in an amount less than about 30% of a Table 1 Column A or Table 2 Column A amount. In one or more embodiments, at least one of the containers 5302 houses a drug in an amount less than about 40% of a Table 1 Column A or Table 2 Column A amount. In one or more embodiments, at least one of the containers 5302 houses a drug in an amount less than about 50% of a Table 1 Column A or Table 2 Column A amount. In one or more embodiments, at least one of the containers 5302 houses a drug in an amount less than about 60% of a Table 1 Column A or Table 2 Column A amount. In one or more embodiments, a plurality of the containers 5302 house a non-standard amount of a drug listed in Table 1 or Table 2.

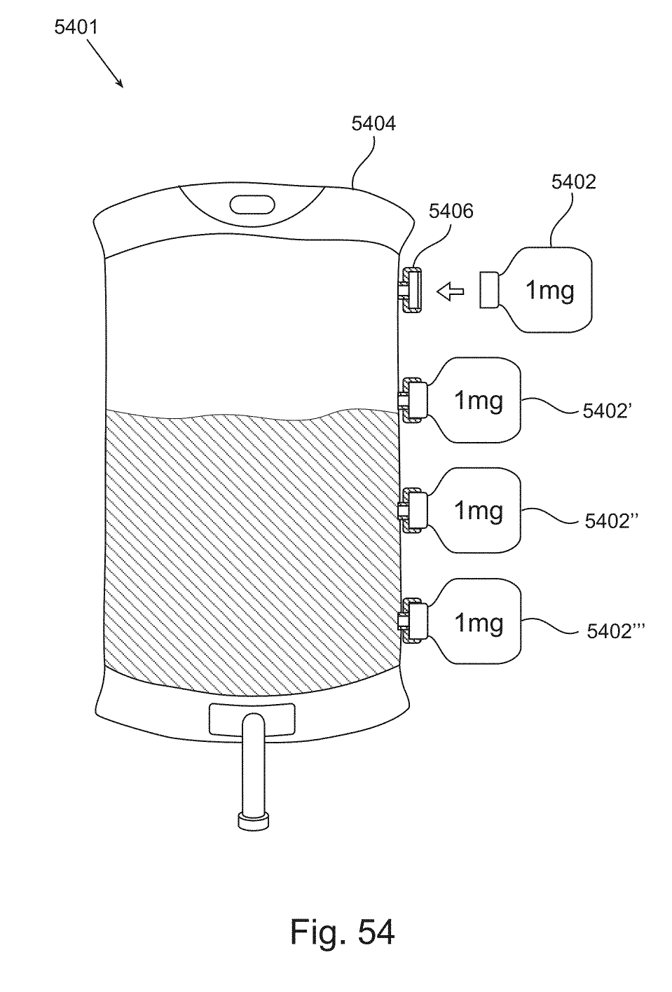

FIG. 54 is a schematic front cut view illustration presenting a further exemplary system 5401 which includes an IV bag 5404 having a plurality of entry ports 5406, wherein a plurality of containers 5402 are received and engaged by the plurality of entry ports 5406 to transfer a dose from the plurality of containers 5402 to the IV bag 5404, according to some embodiments of the invention. The containers 5402 can have different dosing regiments, such as 1 mg, 5 mg, 25 mg or other dosing regiments, so that a user can create non-standard or customized dosing amounts to administer to patients.

In one or more embodiments, the IV bag 5404 may be any vessel or container. In one or more embodiments, the container may be a bottle. In one or more embodiments, the vessel may be a syringe, a manifold or a connector with a plurality of entry ports.

In one or more embodiments, the bottle may have a negative pressure compared to ambient air and/or atmospheric pressure

In one or more embodiments, at least one of the containers 5402 houses a non-standard amount of a beneficial substance and/or drug. In one or more embodiments, a plurality of the containers 5402 houses a non-standard amount of a beneficial substance and/or a drug. Packaging non-standard amounts of a beneficial substance and/or a drug in commercially prepackaged containers 5402 allows for the assembly of a customized and/or individualized amount of a beneficial substance and/or a drug without the need to manually measure and manipulate the beneficial substance. In one or more embodiments, at least one of the containers 5402 houses a drug in an amount less than about 5% of a Table 1 Column A or Table 2 Column A amount. In one or more embodiments, at least one of the containers 5402 houses a drug in an amount less than about 10% of a Table 1 Column A or Table 2 Column A amount. In one or more embodiments, at least one of the containers 5402 houses a drug in an amount less than about 20% of a Table 1 Column A or Table 2 Column A amount. In one or more embodiments, at least one of the containers 5402 houses a drug in an amount less than about 30% of a Table 1 Column A or Table 2 Column A amount. In one or more embodiments, at least one of the containers 5402 houses a drug in an amount less than about 40% of a Table 1 Column A or Table 2 Column A amount. In one or more embodiments, at least one of the containers 5402 houses a drug in an amount less than about 50% of a Table 1 Column A or Table 2 Column A amount. In one or more embodiments, at least one of the containers 5402 houses a drug in an amount less than about 60% of a Table 1 Column A or Table 2 Column A amount. In one or more embodiments, a plurality of the containers 5402 house a non-standard amount of a drug listed in Table 1 or Table 2. In one or more embodiments, the drug may be a beneficial substance. In one or more embodiments, the beneficial substance may be a nutritional substance. In one or more embodiments, the beneficial substance may be a diagnostic substance. In one or more embodiments, the containers 5402 may be commercially packaged containers. In one or more embodiments, the commercially packaged containers may be commercially prepackaged containers. In one or more embodiments, the containers may have tamper evident and/or tamper resistant seals/caps. In one or more embodiments, the commercially prepackaged containers are packaged by a pharmaceutical manufacturer and/or packager.

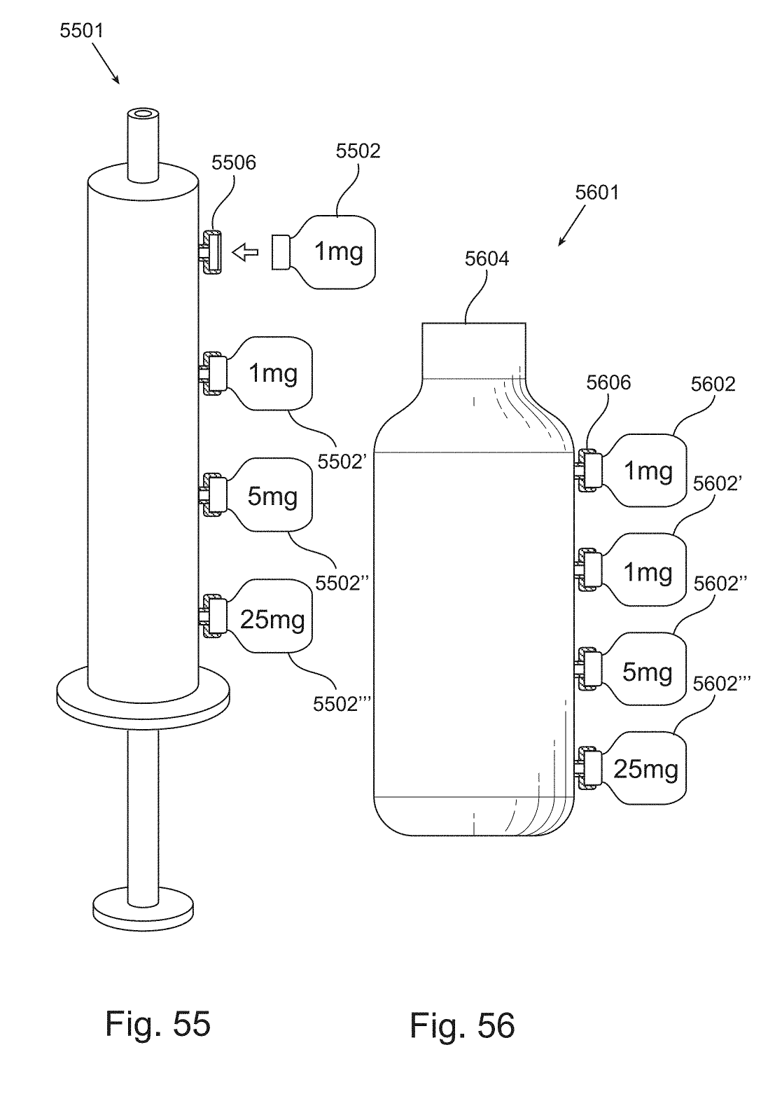

FIG. 55 is a schematic front cut view illustration presenting a further exemplary system which includes a syringe 5501 having a plurality of entry ports 5506, wherein a plurality of containers 5502 are received and engaged by the plurality of entry ports 5506 to transfer a dose from the plurality of containers 5502 to the syringe 5501, according to some embodiments of the invention. The containers 5502 can have different amounts of drugs, such as 1 mg, 5 mg, 25 mg or other amounts, so that a user can create non-standard and/or customized dosing amounts to administer to patients. In one or more embodiments, at least one of the containers 5502 houses a non-standard amount of a beneficial substance and/or drug. In one or more embodiments, a plurality of the containers 5502 houses a non-standard amount of a beneficial substance and/or a drug. Packaging non-standard amounts of a beneficial substance and/or a drug in commercially prepackaged containers 5502 allows for the assembly of a customized and/or individualized amount of a beneficial substance and/or a drug without the need to manually measure and manipulate the beneficial substance. In one or more embodiments, at least one of the containers 5502 houses a drug in an amount less than about 5% of a Table 1 Column A or Table 2 Column A amount. In one or more embodiments, at least one of the containers 5502 houses a drug in an amount less than about 10% of a Table 1 Column A or Table 2 Column A amount. In one or more embodiments, at least one of the containers 5502 houses a drug in an amount less than about 20% of a Table 1 Column A or Table 2 Column A amount. In one or more embodiments, at least one of the containers 5502 houses a drug in an amount less than about 30% of a Table 1 Column A or Table 2 Column A amount. In one or more embodiments, at least one of the containers 5502 houses a drug in an amount less than about 40% of a Table 1 Column A or Table 2 Column A amount. In one or more embodiments, at least one of the containers 5502 houses a drug in an amount less than about 50% of a Table 1 Column A or Table 2 Column A amount. In one or more embodiments, at least one of the containers 5502 houses a drug in an amount less than about 60% of a Table 1 Column A or Table 2 Column A amount. In one or more embodiments, a plurality of the containers 5502 house a non-standard amount of a drug listed in Table 1 or Table 2. In one or more embodiments, the drug may be a beneficial substance. In one or more embodiments, the beneficial substance may be a nutritional substance. In one or more embodiments, the beneficial substance may be a diagnostic substance. In one or more embodiments, the containers 5502 may be commercially packaged containers. In one or more embodiments, the commercially packaged containers may be commercially prepackaged containers. In one or more embodiments, the containers may have tamper evident and/or tamper resistant seals/caps. In one or more embodiments, the commercially prepackaged containers are packaged by a pharmaceutical manufacturer and/or packager.

FIG. 56 is a schematic front cut view illustration presenting a further exemplary system 5601 which includes a bottle 5604 having a plurality of entry ports 5606, wherein a plurality of containers 5602 are received and engaged by the plurality of entry ports 5606 to transfer a dose from the plurality of containers 5602 to the bottle 5604, according to some embodiments of the invention. The containers 5602 can have different amounts of drugs, such as 1 mg, 5 mg, 25 mg or other non-standard amounts, so that a user can create non-standard and/or customized/individualized dosing amounts to administer to patients.

In one or more embodiments, at least one of the containers 5402 houses a non-standard amount of a beneficial substance and/or drug. In one or more embodiments, a plurality of the containers 5602 houses a non-standard amount of a beneficial substance and/or a drug. Packaging non-standard amounts of a beneficial substance and/or a drug in commercially prepackaged containers 5602 allows for the assembly of a customized and/or individualized amount of a beneficial substance and/or a drug without the need to manually measure and manipulate the beneficial substance. In one or more embodiments, at least one of the containers 5602 houses a drug in an amount less than about 5% of a Table 1 Column A or Table 2 Column A amount. In one or more embodiments, at least one of the containers 5602 houses a drug in an amount less than about 10% of a Table 1 Column A or Table 2 Column A amount. In one or more embodiments, at least one of the containers 5602 houses a drug in an amount less than about 20% of a Table 1 Column A or Table 2 Column A amount. In one or more embodiments, at least one of the containers 5602 houses a drug in an amount less than about 30% of a Table 1 Column A or Table 2 Column A amount. In one or more embodiments, at least one of the containers 5602 houses a drug in an amount less than about 40% of a Table 1 Column A or Table 2 Column A amount. In one or more embodiments, at least one of the containers 5602 houses a drug in an amount less than about 50% of a Table 1 Column A or Table 2 Column A amount. In one or more embodiments, at least one of the containers 5602 houses a drug in an amount less than about 60% of a Table 1 Column A or Table 2 Column A amount. In one or more embodiments, a plurality of the containers 5602 house a non-standard amount of a drug listed in Table 1 or Table 2. In one or more embodiments, the drug may be a beneficial substance. In one or more embodiments, the beneficial substance may be a nutritional substance. In one or more embodiments, the beneficial substance may be a diagnostic substance. In one or more embodiments, the containers 5602 may be commercially packaged containers. In one or more embodiments, the commercially packaged containers may be commercially prepackaged containers. In one or more embodiments, the containers may have tamper evident and/or tamper resistant seals/caps. In one or more embodiments, the commercially prepackaged containers are packaged by a pharmaceutical manufacturer and/or packager. In one or more embodiments, the bottle 5604 may be any vessel, container, or medical device. In one or more embodiments, the bottle 5604 may be a bag. In one or more embodiments, the bottle 5604 may be a container with a flexible wall. In one or more embodiments, the bottle 5604 may be a container with a rigid wall.

It should be appreciated that for simplicity and clarity of illustration, elements shown in the figures have not necessarily been drawn to scale. For example, the dimensions of some of the elements are exaggerated relative to each other for clarity. Further, where considered appropriate, reference numerals have been repeated among the figures to indicate corresponding elements.

Each of the following terms: ‘includes’, ‘including’, ‘has’, ‘having’, ‘comprises’, and ‘comprising’, and, their linguistic, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably.

The term ‘about’, is some embodiments, refers to ±30% of the stated numerical value. In further embodiments, the term refers to ±20% of the stated numerical value. In yet further embodiments, the term refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A system for intermixing beneficial substances, comprising:
a syringe having at least two entry ports and an exit port;
at least two containers containing an amount of a medicament;
wherein the at least two entry ports are configured to be received and engaged by the at least two entry ports of the syringe,
wherein upon connection of the at least two containers to the at least two entry ports of the syringe, the amount of the medicament in the at least two containers is transferred into the syringe.

2. The system of claim 1, wherein when the at least two containers are engaged to the at least two entry ports of the syringe, the at least two containers abut the syringe.

3. The system of claim 1, wherein when the at least two containers are engaged to the at least two entry ports of the syringe, the at least two containers abut a wall of the syringe.

4. The system of claim 1, wherein when the at least two containers are engaged to the at least two entry ports of the syringe, the at least two containers are surface mounted to the syringe.

5. The system of claim 1, wherein when the at least two containers are engaged to the at least two entry ports of the syringe, the at least two containers are surface mounted to a wall of the syringe.

6. The system of claim 1, wherein when the at least two containers are engaged to the at least two entry ports of the syringe, the at least two containers are flush mounted to the syringe.

7. The system of claim 1, wherein when the at least two containers are engaged to the at least two entry ports of the syringe, the at least two containers are surface mounted to a wall of the syringe.

8. The system of claim 1, wherein the at least two containers are selected from a group consisting of three containers, four containers, and five containers.

9. The system of claim 1, wherein the at least two entry ports is selected from a group consisting of three entry ports, four entry ports, five entry ports, and six entry ports.

10. The system of claim 1, wherein the at least two containers is selected from a group consisting of a bottle, a vial, a container with an expulsion member and a syringe.

11. The system of claim 1, wherein the syringe includes a rigid surface around the at least two entry ports of the syringe.

12. The system of claim 1, wherein a surface of the syringe that supports the at least two entry ports has a greater rigidity than another surface of the syringe.

13. The system of claim 1, wherein at least one of the at least two entry ports is located on a surface of the syringe and another at least one of the at least two entry ports is located on a different surface of the syringe.

14. The system of claim 1, wherein the at least two entry ports are oriented randomly on one or more surfaces of the syringe.

15. The system of claim 1, wherein the at least two entry ports are oriented on the same surface of the syringe.

16. The system of claim 1, wherein the at least two entry ports are oriented on a side wall of the syringe.
